(19) United States
(12) Patent Application Publication
(10) Pub. No.: US 2021/0181530 A1
(43) Pub. Date: Jun. 17, 2021

(54) MECHANISMS FOR INDUCING TRANSITIONS IN DYNAMIC CONTACT LENSES

(71) Applicant: Pres-By Vision Ltd., Tel Aviv (IL)

(72) Inventors: Yair Alster, Tel Aviv (IL); Omer Rafaeli, Tel Aviv (IL); Nir Betser, Tel Aviv (IL); Matt Clarke, Mountain View, CA (US); Assaf Leon, Tel Aviv (IL)

(21) Appl. No.: 17/169,126

(22) Filed: Feb. 5, 2021

Related U.S. Application Data
(63) Continuation of application No. PCT/IB2019/000956, filed on Sep. 4, 2019.
(60) Provisional application No. 62/726,732, filed on Sep. 4, 2018.

(51) Int. Cl. G02C 7/04

(52) U.S. Cl. CPC G02C 7/047; G02C 2202/24; G02C 7/049

(57) ABSTRACT

Dynamic contact lenses having an optical portion that has at least two quasi-stable configurations. Interaction of the dynamic contact lens with an eyelid and/or the tear meniscus can induce a transition between the quasi-stable configurations are disclosed. A dynamic contact lens can include one or more mechanisms that can facilitate interaction of the contact lens with an eyelid and/or a source of tear fluid such as a tear meniscus and that can facilitate transitioning between the quasi-stable configurations. The mechanisms can be configured to control the flow of tear fluid into and out of a tear volume formed between the posterior surface of the optical portion and the anterior surface of the cornea. The dynamic contact lenses can be used for correcting vision such as for correcting presbyopia, delaying the progression of myopia, or for correcting vision caused by an irregularly-shaped cornea.

### CROSS-REFERENCE TO RELATED APPLICATIONS

This application is a continuation of International Application No. PCT/IB2019/000956, filed Sep. 4, 2019, which claims the benefit of U.S. Provisional Application No. 62/726,732, filed Sep. 4, 2018, which is incorporated herein by reference in its entirety.

### BACKGROUND

Typical vision deficiencies such as myopia (nearsightedness), hyperopia (farsightedness), and presbyopia (loss of accommodation and subsequent loss of near and intermediate vision) may be readily correctable using eyeglasses. However, some individuals may prefer contact lenses for vision correction for reasons such as to accommodate an active life style or for aesthetics.

Contact lens wearers who become presbyopic with age may require additional corrective lenses to allow both near, intermediate, and distance vision. To address presbyopia, contact lens manufacturers have developed multifocal lenses that simultaneously focus light from a range of distances via several focal regions and bifocal lenses that include two focusing regions, e.g., a central region for correcting myopia and a surrounding region for correcting hyperopia. The latter lenses may translate with respect to the optical axis of the eye to provide both near and far vision correction depending on the eye gaze angle.

Translating contact lenses may be configured for moving (translating) anywhere from 1 mm to 6 mm over the surface of the cornea and as such may be significantly less stable than standard contact lenses, which typically have a movement over the cornea from 0 mm to 1 mm. Because translating lenses may be designed to move, during upper eyelid blinking, translating lenses may shift downward over the cornea such that the lower edge of the lens impinges upon the lower lid margin with every blinking motion. Such repeated movement and lid contact may cause significant user discomfort due to the heightened foreign object sensitivity of the cornea and lower lid margin. In addition, due to the presence of the meibomian gland opening on the lower lid margin, lower lid impingement can lead to repeated trauma and inflammation of these openings which can lead to hyperkeratosis and possibly meibomian gland dysfunction.

### SUMMARY

Recognized herein is a need for alternative contact lenses for correcting vision.

In an aspect, the present disclosure provides a contact lens comprising an optical portion, wherein the optical portion comprises an optical posterior base curvature and an optical center, a peripheral portion, wherein the peripheral portion comprises a peripheral posterior base curvature, and a transition zone coupling the optical portion and the peripheral portion. When worn on the eye of a patient, the optical portion is characterized by a first quasi-stable configuration and a second quasi-stable configuration. Interaction of the contact lens with eye movement causes a transition between the first quasi-stable configuration and the second quasi-stable configuration.

In some embodiments, the transition zone comprises a radial width of 150 microns or less.

In some embodiments, the transition zone comprises a circumference and a thickness, wherein the thickness varies around the circumference of the transition zone.

In some embodiments, the optical portion comprises an optical posterior surface and the peripheral portion comprises a peripheral posterior surface and a peripheral anterior surface. The contact lens further comprises one or more grooves in the peripheral posterior surface, wherein at least one groove extends from the peripheral posterior surface to the optical portion; and at least one fenestration connecting the at least one groove to the peripheral anterior surface. The transition zone comprises a circumference and a thickness; the thickness varies around the circumference of the transition zone; the optical posterior base curvature is less than 7.1 mm; and the peripheral posterior base curvature is at least 0.4 mm greater than the optical posterior base curvature at a radius less than 3.5 mm from the optical center.

In some embodiments, the optical portion comprises an optical posterior surface; and the peripheral portion comprises a peripheral diameter, a peripheral posterior surface, and a peripheral anterior surface. The contact lens is configured such that when worn on an eye of a patient, the optical portion forms a lenticular volume between the cornea and the optical posterior surface. The lenticular volume comprises a diameter of at least 1.5 mm and a height of at least 0.01 mm over the cornea.

In some embodiments, the peripheral portion comprises a peripheral diameter; and the contact lens is configured such that, when worn on an eye of a patient, the optical portion is capable of assuming the first quasi-stable configuration and the second quasi-stable configuration.

In some embodiments, the optical portion comprises an optical posterior surface and the peripheral portion comprises a peripheral posterior surface, The contact lens is configured such that when worn on the eye of a patient, the optical portion can assume a plurality of configurations in response to a pressure applied to the optical portion. When a negative pressure is applied to the optical posterior surface, the optical posterior surface assumes one or more substantially conforming configurations with respect to the anterior surface of the cornea. In the absence of a negative pressure, the optical posterior surface assumes a neutral configuration to provide a tear volume between the optical posterior surface and the anterior surface of the cornea. In some embodiments, the in the one or more substantially conforming configurations the thickness of a tear film between the optical posterior surface and the anterior surface of the cornea varies by less than 10 μm. For example, in some embodiments the in the one or more substantially conforming configurations the thickness of a tear film between the optical posterior surface and the anterior surface of the cornea varies by less than 3 μm. In some embodiments, the negative pressure is from 5 Pa to 1,500 Pa. For example, in some embodiments the negative pressure is from 10 Pa to 250 Pa.

In some embodiments, the peripheral posterior base curvature is from 7.5 mm to 9.5 mm and the difference between the peripheral posterior base curvature and the optical posterior base curvature is greater than 0.4 mm.

In some embodiments, the optical posterior base curvature is less than 6.8 mm.

In some embodiments, the transition zone has a thickness that varies around the circumference of the transition zone.

In some embodiments, the transition zone has a thickness that varies in a regular pattern around the circumference of the transition zone.

In some embodiments, the transition zone comprises one or more discontinuities extending across the transition zone. In some embodiments, the one or more discontinuities comprises one or more posterior grooves in the posterior surface of the peripheral portion and extending into the optical portion. In some embodiments, at least one of the one or more posterior grooves are coupled to a fenestration. Alternatively, or in combination, at least one of the one or more posterior grooves are coupled to a tear fluid reservoir.

In some embodiments, the optical posterior base curvature is less than 7.1 mm and the peripheral base curvature is at least 0.4 mm greater than the optical posterior base curvature.

In some embodiments, each of the optical portion and the peripheral portion comprises a material having a modulus from 0.1 MPa to 10 MPa.

In some embodiments, the contact lens comprises one or more posterior grooves in the peripheral posterior surface, wherein at least one posterior groove extends from the peripheral posterior surface into the optical portion.

In some embodiments, each of the one or more grooves extends radially from the center of the optical portion.

In some embodiments, the transition zone is located at a radius less than 3.5 mm from the optical center; the central base curvature is less than 7.1 mm and the peripheral base curvature is at least 0.4 mm greater than the center base curvature.

In some embodiments, the first quasi-stable configuration comprises a first gap height, the second quasi-stable configuration comprises a second gap height, the first gap height and the second gap height are different; and the gap height is the distance between a center of the optical posterior surface and the cornea.

In some embodiments, eye movement comprises changing a gaze position of the eye.

In some embodiments, in the first quasi-stable configuration the optical portion comprises a first optical power and in the second quasi-stable configuration the optical portion comprises a second optical power, wherein the first optical power is different than the second optical power.

In some embodiments, when worn on the eye of a patient, an optical tear volume is formed between the optical posterior surface and the anterior surface of the cornea. In the first quasi-stable configuration the optical tear volume comprises a first volume and in the second quasi-stable configuration the optical tear volume comprises a second volume, wherein the first volume is different than the second volume.

In some embodiments, when worn on the eye of a patient, an optical tear volume is formed between the optical posterior surface and the anterior surface of the cornea. In the first quasi-stable configuration the optical tear volume comprises a first shape, in the second quasi-stable configuration the optical tear volume comprises a second shape, and the first shape is different than the second shape.

In some embodiments, the first quasi-stable configuration provides an optical power that focuses an image on the fovea from a first distance and the second quasi-stable configuration provides an optical power that focuses an image on the fovea from a second distance.

In some embodiments, when worn on the eye of a patient, the optical portion is characterized by a first quasi-stable configuration and a second quasi-stable configuration and an optical tear volume is formed between the optical posterior surface and the anterior surface of the cornea; and a transition between the first quasi-stable configuration and the second quasi-stable configuration is controlled by the flow of tear fluid into and out of the optical tear volume.

In some embodiments, when worn on the eye of a patient, the optical portion is characterized by a first quasi-stable configuration and a second quasi-stable configuration and an optical tear volume is formed between the optical posterior surface and the anterior surface of the cornea; and a transition between the first quasi-stable configuration and the second quasi-stable configuration is controlled by fluidly coupling and decoupling the optical tear volume with a tear meniscus.

In some embodiments, when worn on the eye of a patient, the optical portion is characterized by a first quasi-stable configuration and a second quasi-stable configuration; the contact lens comprises one or more fenestrations connecting the peripheral posterior surface to the anterior posterior surface; and fluidly coupling one or more fenestrations to a tear meniscus causes a change in the optical power of the optical portion.

In some embodiments, when worn on the eye of a patient, the optical portion is characterized by a first quasi-stable configuration and a second quasi-stable configuration; the contact lens comprises one or more fenestrations connecting the peripheral posterior surface to the peripheral anterior surface; and fluidly decoupling one or more fenestrations with a tear meniscus causes a change in the optical power of the optical portion.

In some embodiments, at least one groove in the peripheral posterior surface, wherein the at least one groove extends from the peripheral posterior surface to the optical portion; and at least one fenestration connecting the at least one groove to the peripheral anterior surface.

In some embodiments, when worn on an eye of a patient, an optical tear volume is formed between the posterior surface of the optical portion and the anterior surface of the cornea.

In some embodiments, when worn on an eye of a patient, a gap is formed between the posterior surface of the optical portion and the anterior surface of the cornea, wherein the gap has a maximum height from 1 μm to 200 μm.

In some embodiments, the optical portion is centered on the central axis of the contact lens.

In some embodiments, the optical portion is not centered on the central axis of the contact lens.

In some embodiments, the optical portion is centered on an axis that is less than 45 degrees from the central axis of the contact lens.

In some embodiments, the optical portion comprises a maximum thickness within a range from 30 μm to 600 μm.

In some embodiments, the optical portion comprises a maximum rigidity within a range from 2E3 MPa×μm³ to 3E9 MPa×μm³.

In some embodiments, the optical portion, the peripheral portion, or both the optical portion and the peripheral portion comprise at least one mechanism configured to transport tear fluid into and out of an optical tear volume formed between the optical posterior surface and the anterior surface of the cornea, when worn on an eye of a patient. In some embodiments, the transport of tear fluid into and out of the optical tear volume is associated with a transition between the first quasi-stable configuration of the optical portion and the second quasi-stable configuration of the optical portion. In some embodiments, the at least one mechanism comprises a posterior groove, an anterior groove, a fenestration, a tear fluid reservoir, a protrusion, a depression, a valve, a fenestration comprising a valve, a geometry of the optical portion, a geometry of the peripheral portion, or a combination of any of the foregoing. In some embodiments, the at least one mechanism comprises one or more posterior grooves, wherein each of the one or more posterior grooves is disposed in the peripheral posterior surface. In some embodiments, at least one of the one or more posterior grooves intersects the circumference of the optical portion. In some embodiments, the at least one mechanism is disposed within the peripheral portion, on the posterior surface of the peripheral portion, on the anterior surface of the peripheral portion, or a combination of any of the foregoing. In some embodiments, the at least one mechanism comprises a protrusion on the peripheral anterior surface.

In some embodiments, interaction of tear fluid in the tear meniscus with the optical tear volume induces a transition between the first quasi-stable configuration of the optical portion and the second quasi-stable configuration of the optical portion, maintains the first quasi-stable configuration of the optical portion, maintains the second quasi-stable configuration of the optical portion, or a combination of any of the foregoing.

In some embodiments, motion of the eye, an eyelid, or a combination thereof, induces a transition between the first quasi-stable configuration of the optical portion and the second quasi-stable configuration of the optical portion, maintains the first quasi-stable configuration of the optical portion, maintains the second quasi-stable configuration of the optical portion, or a combination of any of the foregoing.

In some embodiments, interaction of tear fluid in the tear meniscus with at least two of the optical portion, the peripheral portion, and the at least one mechanism, induces a transition between the first quasi-stable configuration of the optical portion and the second quasi-stable configuration of the optical portion, maintains the first quasi-stable configuration of the optical portion, maintains the second quasi-stable configuration of the optical portion, or a combination of any of the foregoing

In some embodiments, interaction between tear fluid within the optical tear volume and tear fluid within a tear fluid source induces a transition between the first quasi-stable configuration of the optical portion and the second quasi-stable configuration of the optical portion, maintains the first quasi-stable configuration of the optical portion, maintains the second quasi-stable configuration of the optical portion, or a combination of any of the foregoing. In some embodiments, the tear fluid source comprises a tear fluid reservoir, a tear fluid depression, a tear meniscus, or a combination of any of the foregoing. In some embodiments, interaction is induced by a change in gaze angle, by interaction of an eyelid with the contact lens, or by a combination thereof. In some embodiments, interaction comprises fluidly coupling and fluidly decoupling the optical tear volume with a tear fluid source. In some embodiments, interaction comprises fluidly coupling and fluidly decoupling the optical tear volume with a tear meniscus.

In some embodiments, the contact lens further comprises at least one fenestration connecting the peripheral posterior surface to the anterior posterior surface; and at least one of the fenestrations comprises a valve. In some embodiments, the valve comprises a capillary valve.

In some embodiments, the contact lens further comprises one or more anterior grooves disposed in the peripheral anterior surface and one or more fenestrations connected to each of the one or more anterior grooves, wherein the at least one fenestration connects the anterior groove to the peripheral posterior surface. In some embodiments, the contact lens comprises a posterior groove disposed in the peripheral posterior surface and connected to at least one of the one or more fenestrations. In some embodiments, at least one of the one or more posterior grooves extends into the optical portion.

In some embodiments, the contact lens further comprises a plurality of radially disposed posterior grooves; and one or more fenestrations, wherein one or more fenestrations is coupled to each of the plurality of radially disposed posterior grooves.

In some embodiments, the contact lens further comprises one or more depressions disposed in the anterior peripheral surface, and a fenestration coupled to each of the one or more depressions. In some embodiments, the fenestration is coupled to a posterior groove.

In some embodiments, the peripheral portion comprises a cavity disposed in the peripheral posterior surface. In some embodiments, the cavity is deformable upon interaction with an eyelid, motion of the eye, or a combination thereof.

In some embodiments, the peripheral portion comprises a depression in the peripheral anterior surface; a fenestration coupled to the depression; and a posterior groove coupled to the fenestration, wherein the posterior groove extends into the optical portion.

In an aspect, the present disclosure provides a method of correcting vision, the method comprising wearing, or providing to a wearer, any of the contact lenses described herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a cross-sectional view of a dynamic contact lens provided by the present disclosure.

FIGS. 2A-2B show examples of fish-mouth valves provided by the present disclosure.

FIGS. 3A-3C illustrate parameters useful in calculating capillary forces.

FIG. 3D shows a cross-sectional view of a capillary meniscus formed within a fenestration caused by capillary forces.

FIGS. 4A-4B show a dynamic model of fluid transport in an example of a dynamic contact lens provided by the present disclosure.

FIGS. 5A-5B show a dynamic model of fluid transport in another example of a dynamic contact lens provided by the present disclosure.

FIGS. 6A-6B show views of a dynamic contact lens having an abrupt transition zone and discontinuities around the circumference of the transition zone.

FIGS. 7A-7D show a view of a dynamic contact lens having an abrupt transition zone, and views of the transition zone.

FIGS. 8A-8C show views of a dynamic contact lens having discontinuities in the transition zone.

FIGS. 9A-91 show views of a dynamic contact lens having discontinuities in the transition zone.

FIG. 10 shows a view of the posterior surface of an example of a dynamic contact lens provided by the present disclosure with grooves extending from the peripheral posterior surface to the dynamic portion and with fenestrations connected to each of the grooves.

FIG. 11 shows a view of the anterior surface of the dynamic contact lens shown in FIG. 10.

FIG. 12 shows a view of the posterior surface of an example of a dynamic contact lens provided by the present disclosure.

FIGS. 13A and 13B show a cross-sectional view and a view of the posterior surface, respectively, of an example of a dynamic contact lens provided by the present disclosure.

FIG. 13C shows an image of the dynamic contact lens of FIGS. 13A-13B on an eye of a patient.

FIG. 14 shows a slit lamp bio-microscope image of a dynamic contact lens having eight (8) fenestrations on an eye of a patient

FIGS. 15A-15H show views of a dynamic contact lens having depressions and fenestrations within the depressions disposed in the second peripheral portion near the transition zone.

FIGS. 16A-16C show perspective views of the anterior surface (FIG. 16A), the posterior surface (FIG. 16B), and a cross-sectional view (FIG. 16C) of an example of a dynamic contact lens having an elongated anterior groove configured to fluidly couple with a tear meniscus and a fenestration and posterior groove for transporting tear fluid to the optical tear volume.

FIGS. 17A-17D show views of the anterior surface (FIGS. 17A and 17B) and the posterior surface (FIGS. 17C and 17D) of examples of dynamic contact lenses having a plurality of fenestrations disposed at different radial distances from the optical center and posterior grooves for transporting tear fluid from a tear meniscus to the optical tear volume.

FIGS. 18A-18C show perspective views of the anterior surface (FIG. 18A), the posterior surface (FIG. 18B), and a cross-sectional view (FIG. 18C) of an example of a dynamic contact lens having an anterior groove configured to fluidly couple with a tear meniscus and with a fenestration and posterior groove for transporting tear fluid to the optical tear volume.

FIGS. 19A-19C show perspective views of the anterior surface (FIG. 19A), the posterior surface (FIG. 19B), and a cross-sectional view (FIG. 19C) of an example of a dynamic contact lens having anterior grooves configured to fluidly couple with a tear meniscus and fenestrations and posterior grooves for transporting tear fluid to the optical tear volume.

FIGS. 20A and 20B show OCT images of a dynamic contact lens including fenestrations and grooves on the cornea of a patient. FIG. 20A shows a fenestration fluidly coupled to a tear meniscus. FIG. 20B shows the groove tapering toward the optical portion.

FIGS. 21A and 21B show horizontal (FIG. 21A) and vertical (FIG. 21B) OCT images of a dynamic contact lens on the cornea of a patient. FIG. 21B shows a fenestration fluidly coupled to a tear meniscus.

FIG. 21C shows an OCT image of a dynamic contact lens on a cornea showing fluid coupling of a tear meniscus to a groove with a gap height of 68 μm between the posterior surface of the optical portion and the anterior surface of the cornea.

FIG. 22 shows an OCT image of a tear volume formed between the posterior surface of the optical portion of a dynamic contact lens and the anterior surface of the cornea.

FIG. 23 is a slit lamp bio-microscope image of a dynamic contact lens overlying a cornea with the eye in a downward gaze.

FIG. 24 shows an OCT images of a dynamic contact lens overlying a cornea with forward gaze.

FIG. 25 shows an OCT images of a dynamic contact lens overlying a cornea with forward gaze after tear fluid has been provided to the tear volume through a fenestration and posterior groove.

FIG. 26 shows an OCT image of dynamic contact lens overlying a cornea with a gap of about 10 μm to 15 μm between the posterior surface of the optical portion and the cornea.

FIG. 27 shows an OCT image of the peripheral portion of the contact lens shown in FIG. 26 with a posterior groove.

FIG. 28 is an OCT image of a dynamic contact lens overlying a cornea showing a cross-sectional view of a posterior groove.

FIG. 29 is an OCT image of a dynamic contact lens overlying a cornea in downgaze showing a cross-sectional view of a fenestration coupled to a posterior groove

FIG. 30 is an OCT image of a dynamic contact lens overlying a cornea showing a cross-sectional view of the optical portion and the optical tear volume during downward gaze.

FIG. 31 is an OCT image of a dynamic contact lens overlying a cornea showing a cross-sectional view of a posterior groove during downward gaze.

FIG. 32 shows a dynamic contact lens having anterior grooves of various lengths to facilitate fluid coupling with a tear meniscus.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, like characters refer to like elements.

As used herein, the term “posterior” describes features facing toward the eye and the term “anterior” describes features facing away from the eye when worn by a patient. A posterior surface of a dynamic contact lens or portion thereof refers to a surface that is near to or faces the cornea during wear by a patient. The anterior surface of a dynamic contact lens or portion thereof refers to a surface that is away from or faces away from the cornea during wear by a patient.

As used herein, the term “interaction of an eyelid with the dynamic contact lens” refers to any movement of the eyelid or the eyeball that changes the relative position between the dynamic contact lens and either of the eyelids. The interaction includes, for example, smooth sliding of an eyelid over the anterior surface of the dynamic contact lens and a change in position of the dynamic contact lens with respect to a tear meniscus. Interaction of an eyelid or change in gaze angle can couple tear fluid in a tear meniscus with other contact lens features such as fenestrations, peripheral posterior grooves, and/or peripheral anterior grooves. Interaction also refers to translating the dynamic contact lens caused by eye movement and deforming the dynamic contact lens caused by eye movement. For example, during eye movement such as during downward gaze, different areas of a dynamic contact lens can come into contact with an eyelid.

As used herein, the term “interaction of tear meniscus with the dynamic contact lens” refers to any interaction of a tear meniscus with an area or feature of a dynamic contact lens of the lens such as a fenestration, a peripheral posterior groove, and/or a peripheral anterior groove. Interaction of the dynamic contact lens with a tear meniscus can fluidly couple and decouple the tear meniscus to the optical tear volume.

As used herein, the term “optical tear volume” refers to the tear volume between the posterior surface of the optical portion and the anterior surface of the cornea with the dynamic contact lens is worn on the eye of a patient. The optical tear volume can be a lenticular tear volume or in a substantially conforming configuration, can be a tear film having a substantially constant thickness across the optical portion. The optical lens system includes the optical portion of the dynamic contact lens, the tear film, and the lenticular optical tear volume, if present.

As used herein, the term “substantially” refers to ±10% of a value such as a dimension.

As used herein, the term “substantially conforming to the surface of the cornea” refers to a configuration in which the posterior surface of a portion of a dynamic contact lens is within 3 μm from the surface of the cornea. The gap between the posterior portion of the dynamic contact lens and the cornea can comprise tear fluid.

As used herein, the term “modulus” of refers to the Young's modulus of a material. The Young's modulus can be determined, for example, according to the method described by Jones et al., Optometry and Vision Science, 89, 10, 1466-1476, 2017, which is incorporated herein by reference in its entirety for all purposes.

The optical power of the cornea in diopters (D) can be related to the radius of curvature R by the formula D=(1.376−1)/R, where 1.376 corresponds to the index of refraction of the cornea and R corresponds to the radius of curvature of the anterior surface of the cornea. The curvature of the cornea is inversely related to the radius of curvature R such that as the radius of curvature increases the curvature of the cornea decreases, and such that as the radius of curvature decreases the curvature of the cornea increases.

Although rigid gas permeable (RGP) lenses are known to create lenticular tear volumes, RGP lenses do not possess the ability to change conformation. Soft contact lenses typically conform to the corneal surface in a uniform manner and although a 1 μm- to 5 μm-thick tear film is typically present between the posterior surface of the soft contact lens and the cornea, the tear film is not used and does not have a thickness not sufficient to substantially contribute to optical power. Bimodulus contact lenses include a center optical portion having a higher rigidity than the peripheral portion such that the center optical portion vaults over irregularities of the optical portion of the cornea. However, the center optical portion is not dynamic in the sense that it can change conformation during wear. Also, contact lenses having a higher rigidity than typical soft contact lenses require that the contact lens be fit to a particular corneal base curve. In the present invention, a dynamic tear volume is used in conjugation with soft contact lens materials to provide an optical tear volume that can change configuration during wear.

Dynamic contact lenses provided by the present disclosure can be fabricated with an optical portion that can transition between two or more quasi-stable configurations on the eye where each of the two or more quasi-stable configurations provides a different optical power. The difference in optical power between the two quasi-stable configurations is primarily determined by the difference in the refractive power of the optical anterior surface of the optical portion of the dynamic contact lens. When in a quasi-stable configuration in which the optical portion or at least a part of the optical portion does not conform to the cornea, a lenticular volume is formed between the anterior surface of the cornea and the posterior surface of the optical portion of the dynamic contact lens which can fill with tear fluid to form an optical tear volume that, in conjunction with other optical elements of the dynamic contact lens, provides an optical power for correcting vision. Dynamic contact lenses can be configured to transition between a quasi-stable conforming configuration and one or more quasi-stable non-conforming configurations.

Alternatively, or in combination, a dynamic contact lens can comprise two or more quasi-stable configurations. A quasi-stable configuration refers to a configuration of a dynamic contact lens which is stable in the absence of a force applied to the contact lens by an eye lid, by coupling with a source of tear fluid such as a tear meniscus, and/or by decoupling from a source of tear fluid available to fill the optical tear volume. Interaction of the dynamic contact lens with an eyelid or movement of the eye can cause a quasi-stable configuration to destabilize and can result in the optical portion of the dynamic contact lens transitioning to another quasi-stable configuration. For example, interaction of the dynamic contact lens with a source of tear fluid such as a tear meniscus can stabilize and/or destabilize the one of the quasi-stable configuration by providing tear fluid to or removing tear fluid from the optical tear volume. Dynamic lenses can be configured to transition between two or more quasi-stable configurations.

Dynamic contact lenses provided by the present disclosure can be fabricated with an optical portion that can transition between two or more quasi-stable configurations on the eye where each of the two or more quasi-stable configurations provides a different optical power. When in a quasi-stable configuration in which the optical portion or at least a part of the optical portion does not conform to the cornea, the anterior surface of the optical portion maintains a curvature that is different than that of other quasi-stable configurations and a lenticular volume is formed between the anterior surface of the cornea and the posterior surface of the optical portion of the dynamic contact lens which can fill with tear fluid to form a tear volume that can change the shape of the optical lens elements. In this case, the at least two quasi-stable configurations are both non-conforming such that in each quasi-stable configuration the optical anterior surface has a different anterior curvature and therefore each quasi-stable configuration provides a different optical power to the eye.

For a dynamic contact lens, four optical interfaces contribute to the optical power of the optical system in the various quasi-stable configurations: (1) the air-tear interface, (2) the tear-lens interface, (3) the lens-tear interface, and (4) the tear-cornea interface. All of the optics posterior to the cornea will remain substantially uniform in presbyopic patients. The refractive power at any of these optical interfaces can be calculated using the following equation:

Power (D)=(n₂−n₁)/R_{c}

where n₂ is the refractive index of the material on the posterior side of an interface, n₁ is the material on the anterior side of the interface, and R_{c} is the radius of curvature of the interface in meters. To calculate the optical contribution of a given medium within the optical system, the optical power of the anterior and posterior surfaces of the medium can be added. The equation provides a reasonable estimated provided that thickness of the medium is negligible compared to the radius of curvature of the interfaces, which is valid for the optical systems including a dynamic optical lens provided by the present disclosure.

For a cylindrically-shaped optical surface, the quantitative relationships can be calculated for each meridian.

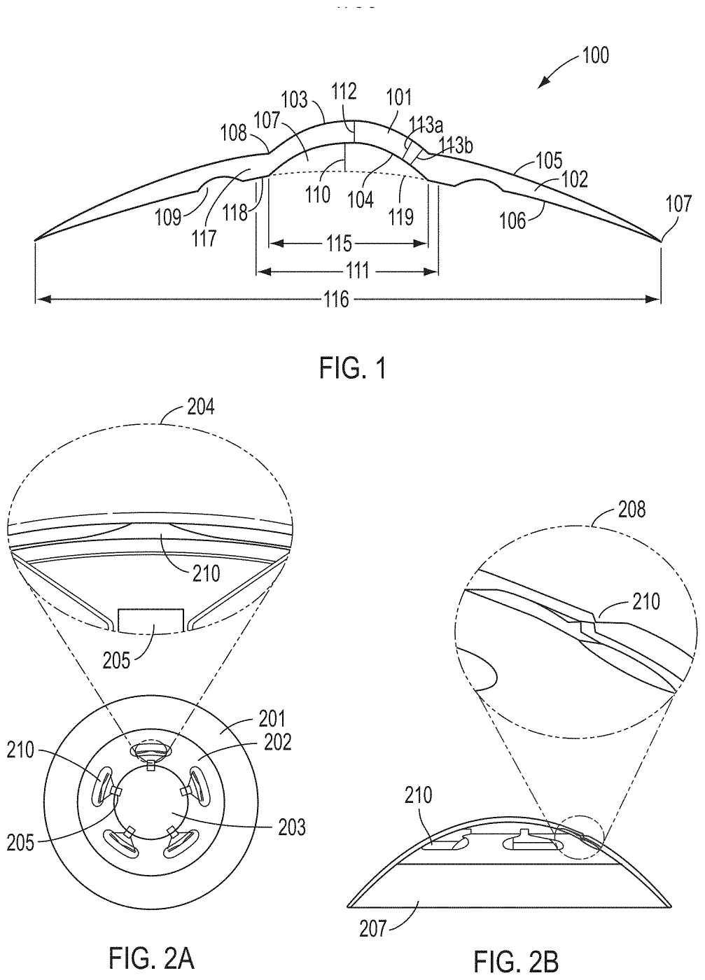

A cross-section of an example of a dynamic contact lens 100 provided by the present disclosure is shown in FIG. 1. The lens 100 includes an optical portion 101 that bulges away from the peripheral posterior base curvature of a peripheral posterior surface 106 of a peripheral portion 102 and/or bulges away from the peripheral base curvature of the peripheral portion 117 adjacent the optical portion 101. This region of the peripheral portion can be referred to as the paracentral peripheral portion or the transition zone 117 which is adjacent the optical portion 101. The posterior surface 118 of the paracentral peripheral portion 117 has a paracentral base curvature. The paracentral base curvature of the posterior surface 118 of the paracentral peripheral portion 117, also referred to as the transition zone, can be the same as the base curvature as posterior surface 106, or can have a different base curvature. For example, the paracentral base curvature of the posterior surface 118 of the paracentral peripheral portion 117 can be greater than the peripheral base curvature. In an as-fabricated and non-conforming configuration, the optical portion 101 bulges away from the base curvature of the paracentral peripheral portion and from the peripheral posterior base curvature 119. It should be appreciated that the peripheral posterior base curvature 119 of peripheral portion 102 represents a base curvature that is different from the posterior base curvature of dynamic optical portion 101 (referred to as the optical posterior base curvature) and each of peripheral portion 102 and optical portion 101 can comprise one or more base curvatures. Optical portion 101 and peripheral portion 102 are coupled at interface 108.

As shown in FIG. 1, the transition zone 108 can be abrupt. In some embodiments, an abrupt transition can provide structural strength to the optical portion 101. In other embodiments, the transition zone 108 can provide a seal between the posterior surface of the contact lens and the anterior surface of the cornea to prevent tear fluid from leaking into or out of the optical tear volume.

In certain embodiments, the transition zone between the peripheral portion and/or the paracentral portion 102/117 and the optical portion 101 is not abrupt. The peripheral posterior surface can comprise cavities 109, which when placed on the cornea fill with tear fluid to provide tear fluid reservoirs. The peripheral posterior surface 106 comprises a peripheral posterior base curvature. The extension of the peripheral posterior base curvature 119 under the region of the optical portion 101 is indicated by the dashed line 119. A sagittal height 110 is shown as the distance from the peripheral base curvature to the posterior surface of the lens. As used herein, the sagittal height refers to a dimension of the as-fabricated dynamic contact lens and can be referred to as the as-fabricated sagittal height. When a dynamic contact lens is applied to the cornea, the distance between the posterior surface of the optical portion and the cornea is referred to the gap height. As disclosed herein, the gap height may be the same as the S sagittal height, however, in many embodiments, on the eye of the patient the gap height is less than the as-fabricated sagittal height. At some gaze angles, the gap height can be less than the as-fabricated sagittal height and at other gaze angles, the gap height can be close to the as-fabricated sagittal height. The center bulge comprises a plurality of sagittal heights depending on the radial distance from the center of the lens.

In FIG. 1 the largest sagittal height is at the center of the optical portion which is located at the center geometric axis of the lens 112. The sagittal height decreases toward the periphery of the optical portion 115 forming a lens shape. In FIG. 1, the optical region 111 is slightly larger than the diameter of the optical portion. When worn on the eye of a patient the distance 110 is referred to as the gap height and is the distance between the posterior surface of the optical portion (the optical posterior surface) and the anterior surface of the cornea. The optical portion refers to the portion of the lens used for vision. The diameter of the optical portion can be larger than that of the optical region of the eye. In some embodiments, the diameter of the optical portion can be less than the diameter of the optical region of the eye. In some embodiments the diameter of the optical portion can be similar to, the same as, or larger than the diameter of the optical region of the eye.

As shown in FIG. 1, the center sagittal height 110 is defined as the as-fabricated distance between the extended curvature of the peripheral posterior surface 106 which is configured to lie against the cornea and the posterior surface at the center of optical portion 104. The optical portion can be characterized by a plurality of sagittal heights depending on the location with respect to the center axis of the bulging optical portion. The sagittal height will be a maximum in the center and will decrease toward the periphery of the optical portion. The optical portion 101 comprises a center thickness 112 and examples of two radial sagittal thickness are identified as 113a and 113b. In FIG. 1 the diameter of the optical region 111 is shown as being slightly larger than the diameter 115 of the optical portion. The dynamic contact lens 100 has a diameter 116. As shown in FIG. 1 the optical portion 101, the peripheral portion 102, and the optical region of the eye can be co-aligned about the center geometric axis of the dynamic contact lens.

Dynamic contact lenses provided by the present disclosure can comprise a peripheral portion comprising a peripheral posterior surface and a peripheral anterior surface opposite the peripheral posterior surface; an optical portion; a transition zone coupling the peripheral portion and the optical portion; wherein the optical portion comprises a material having a Young's modulus, for example, within a range from 0.05 MPa to 50 MPa; and wherein the optical portion is characterized by a cross-sectional profile that extends away from the peripheral anterior surface and away from the peripheral posterior surface. The optical portion may be characterized by an as-fabricated sagittal height of at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The optical portion may be characterized by an as-fabricated sagittal height of at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. The optical portion may be characterized by an as-fabricated sagittal height that is within a range defined by any two of the preceding values. The optical portion can be characterized by an as-fabricated sagittal height within a range, for example, from 10 μm to 250 μm such as from 10 μm to 100 μm. The Young's modulus may be at least about 0.1 MPa, 0.2 MPa, 0.3 MPa, 0.4 MPa, 0.5 MPa, 0.6 MPa, 0.7 MPa, 0.8 MPa, 0.9 MPa, 1 MPa, 2 MPa, 3 MPa, 4 MPa, 5 MPa, 6 MPa, 7 MPa, 8 MPa, 9 MPa, 10 MPa, or more. The Young's modulus may be at most about 10 MPa, 9 MPa, 8 MPa, 7 MPa, 6 MPa, 5 MPa, 4 MPa, 3 MPa, 2 MPa, 1 MPa, 0.9 MPa, 0.8 MPa, 0.7 MPa, 0.6 MPa, 0.5 MPa, 0.4 MPa, 0.3 MPa, 0.2 MPa, 0.1 MPa, or less. The Young's modulus may be within a range defined by any two of the preceding values. The Young's modulus can be within a range, for example, from 0.1 MPa to 20 MPa, from 0.1 MPa to 3 MPa, from 0.1 MPa to 2 MPa, or from 0.1 MPa to 5 MPa. The optical portion may comprise a maximum thickness of at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The optical portion may comprise a maximum thickness of at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. The optical portion may comprise a maximum thickness that is within a range defined by any two of the preceding values. The optical portion can comprise a maximum thickness within a range, for example, from 20 μm to 600 μm, from 50 μm to 500 μm, from 100 μm to 400 μm, or from 50 μm to 300 μm. The optical portion may comprise a center thickness of at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The optical portion may comprise a center thickness of at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. The optical portion may comprise a center thickness that is within a range defined by any two of the preceding values. The optical portion can comprise a center thickness within a range, for example, from 20 μm to 600 μm, from 50 μm to 500 μm, from 100 μm to 400 μm, or from 50 μm to 300 μm. The optical portion can be characterized by a substantially uniform thickness, by a center thickness that is the same as a thickness at the transition zone, by a center thickness that is greater than a thickness at the transition zone, or by a center thickness that is less than a thickness at the transition zone. In other words, the thickness of the optical portion can increase toward the center of the optical portion, can decrease toward the center of the optical portion, or can be substantially constant throughout.

The optical portion can be characterized by an as-fabricated abrupt transition at the interface between the optical portion and the peripheral portion of the lens. The optical portion can have a diameter, for example, of at most about 10 mm, 9 mm, 8 mm, 7 mm, 6 mm, 5 mm, 4 mm, 3 mm, 2 mm, 1 mm, or less. The optical portion may have a diameter of at least about 1 mm, 2 mm, 3 mm, 4 mm, 5 mm, 6 mm, 7 mm, 8 mm, 9 mm, 10 mm, or more. The optical portion may have a diameter that is within a range defined by any two of the preceding values. The interface with the peripheral portion can be characterized by an abrupt change between the peripheral base curve radius of, for example, from 7.5 mm to 8.5 mm to the smaller (steeper) base curve radius of the optical portion. The difference between the two base curve radii may be at least about 0.1 mm, 0.2 mm, 0.3 mm, 0.4 mm, 0.5 mm, 0.6 mm, 0.7 mm, 0.8 mm, 0.9 mm, 1.00 mm, or more. The difference between the two base curve radii may be at most about 1.0 mm, 0.9 mm, 0.8 mm, 0.7 mm, 0.6 mm, 0.5 mm, 0.4 mm, 0.3 mm, 0.2 mm, 0.1 mm, or less. The difference between the two base curve radii may be within a range defined by any two of the preceding values. For example, the difference between the two base curve radii can be greater than 0.2 mm, greater than 0.4 mm, greater than 0.6 mm, or greater than 0.8 mm.

The transition zone can be defined by parameters such as the radial width, the thickness, the base curvature, and/or embedded features. Functionally, the transition zone can be configured to facilitate transport of tear fluid into and out of the optical tear volume, can be configured to facilitate transitions between quasi-stable configurations, and/or can be configured to maintain quasi-stable configurations. The transition zone can be configured to be flexible or rigid compared to the adjacent optical portion and/or the adjacent peripheral portion.

The transition zone is physically coupled to the optical portion and to the peripheral portion. The interface with the optical portion may be situated at a radial distance from the center of the optical portion of at least about 1 mm, 2 mm, 3 mm, 4 mm, 5 mm, 6 mm, 7 mm, 8 mm, 9 mm, 10 mm, or more. The interface with the optical portion may be situated at a radial distance from the center of the optical portion of at most about 10 mm, 9 mm, 8 mm, 7 mm, 6 mm, 5 mm, 4 mm, 3 mm, 2 mm, 1 mm, or less. The interface with the optical portion may be situated at a radial distance from the center of the optical portion that is within a range defined by any two of the preceding values. For instance, the interface with the optical portion can be situated at a radial distance of from 2 mm to 7 mm from the center of the optical portion. The transition zone can have a width, defined as the distance between the interface with the dynamic optical portion and the peripheral portion. The transition zone may have a width of at least about 0 mm, 0.01 mm, 0.02 mm, 0.03 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, or more. The transition zone may have a width of at most 10 mm, 9 mm, 8 mm, 7 mm, 6 mm, 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, or 0 mm. The transition zone may have a width that is within a range defined by any two of the preceding values. The transition zone may have a width, for example, from 0 mm to 0.8 mm, such as from 0.05 mm to 6 mm, from 0.1 mm to 0.5 mm, or from 0.1 mm to 0.4 mm. A transition zone with a width substantially more than 0 mm can have a fillet that has a base curvature that is different than the optical posterior base curvature, the peripheral posterior base curvature, and/or a paracentral posterior base curvature.

An abrupt transition zone refers to a transition zone having no width. In a dynamic contact lens having an abrupt transition zone the optical portion and the peripheral portion are physically coupled without an intermediate width or an intermediate base curvature. An abrupt transition zone may have a width of at least about 0 mm, 0.01 mm, 0.02 mm, 0.03 mm, 0.04 mm, 0.05 mm, 0.06 mm, 0.07 mm, 0.08 mm, 0.09 mm, 0.1 mm, or more. An abrupt transition zone may have a width of at most about 0.1 mm, 0.09 mm, 0.08 mm, 0.07 mm, 0.06 mm, 0.05 mm, 0.04 mm, 0.03 mm, 0.02 mm, 0.01 mm, or 0 mm. An abrupt transition zone may have a width that is within a range defined by any two of the preceding values. An abrupt transition zone can have a width for example, less than 0.1 mm, or less than 0.05 mm.

Across the width of the transition zone the thickness can be the same as the adjacent peripheral portion, different than the thickness of the adjacent peripheral portion, the same as the thickness of the adjacent optical portion, and/or different than the adjacent optical portion. The thickness of the transition zone can be greater or less than the thickness of adjacent portions of the contact lens, i. e, the optical portion and the peripheral portion. The thickness over the width of the transition zone can be constant or can vary.

Across the width of the transition zone, the posterior surface can be characterized by one or more radii of curvature. For example, the posterior surface of the transition zone can have a radius of curvature that is less than that of the adjacent peripheral portion and less than that of the adjacent optical portion; or the transition zone can have a radius of curvature that is less than that of the adjacent peripheral portion and greater than that of the adjacent optical portion.

The transition zone can be transected by posterior grooves, anterior grooves, slits, and/or fenestrations. Thus, the transition zone can be continuous or discontinuous. In a discontinuous transition zone there will be features that interrupt a smooth continuous contact between the perimeter of the dynamic optical portion and the cornea. The discontinuity or discontinuities can serve to reduce the adhesion force at the optical portion against the cornea and thereby can break the interface. For example, the as-fabricated sagittal height can generate a suction force that can cause the perimeter of the optical portion to form a tight seal against the cornea as the center of the optical portion pulls away from the cornea. To reduce the suction force and facilitate the ability to dynamical control the configuration of the optical portion, one or more discontinuities or breaks can be disposed around the circumference of the transition zone. The discontinuities can be coupled to one or more sources of tear fluid such as a tear meniscus.

The transition zone can be characterized by a rigidity that is the same as or is different than a rigidity of the adjacent optical portion and the adjacent peripheral portion.

Around the circumference of the transition zone, the thickness, radius of curvature, and width can be substantially the same, or can be different.

The peripheral portion can comprise a transition zone coupled to the optical portion and the peripheral portion that is characterized by an intermediate radius of curvature; and a distal portion coupled to the intermediate portion characterized by a distal radius of curvature, wherein the intermediate radius of curvature is less than the distal radius of curvature. The transition zone can comprise one or more features configured to facilitate transitioning the dynamic optical portion between two or more quasi-stable configurations and/or maintaining the dynamic optical portion in the two or more quasi-stable configurations.

Dynamic contact lenses provided by the present disclosure can comprise an optical portion comprising an optical posterior surface and an optical anterior surface opposite the optical posterior surface; a peripheral portion comprising a peripheral posterior surface, a peripheral anterior surface opposite the peripheral posterior surface, and a transition zone coupling the peripheral portion and the optical portion; wherein the optical portion comprises a material having a Young's modulus, for example, within a range from 0.05 MPa to 10 MPa; and an as-fabricated center sagittal height, for example, from 10 μm to 300 μm.

The material forming the optical portion may have a Young's modulus of at least about 0.1 MPa, 0.2 MPa, 0.3 MPa, 0.4 MPa, 0.5 MPa, 0.6 MPa, 0.7 MPa, 0.8 MPa, 0.9 MPa, 1 MPa, 2 MPa, 3 MPa, 4 MPa, 5 MPa, or more. material forming the optical portion may have a Young's modulus of at most about 5 MPa, 4 MPa, 3 MPa, 2 MPa, 1 MPa, 0.9 MPa, 0.8 MPa, 0.7 MPa, 0.6 MPa, 0.5 MPa, 0.4 MPa, 0.3 MPa, 0.2 MPa, 0.1 MPa, or less. material forming the optical portion may have a Young's modulus that is within a range defined by any two of the preceding values. The material forming the optical portion can have a Young's modulus, for example, within a range from 0.05 MPa to 8 MPa, from 0.1 MPa to 6 MPa, from 0.1 MPa to 4 MPa, from 0.1 MPa to 3 MPa, from 0.1 MPa to 2 MPa, or from 0.5 MPa to 1 MPa.

The central sagittal height, such as the center as-fabricated sagittal height of the optical portion, may be at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The central sagittal height, such as the center as-fabricated sagittal height of the optical portion, may be at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. The central sagittal height, such as the center as-fabricated sagittal height of the optical portion, may be within a range defined by any two of the preceding values. The center sagittal height, such as the center as-fabricated sagittal height of the optical portion, can be within a range, for example, from 20 μm to 300 μm, from 50 μm to 300 μm, from 10 μm to 200 μm, from 10 μm to 100 μm, from 50 μm to 250 μm, or from 50 μm to 200 μm.

The optical portion may exhibit a maximum rigidity of at least about 1E3 MPa×μm³, 2E3 MPa×μm³, 3E3 MPa×μm³, 4E3 MPa×μm³, 5E3 MPa×μm³, 6E3 MPa×μm³, 7E3 MPa×μm³, 8E3 MPa×μm³, 9E3 MPa×μm³, 1E4 MPa×μm³, 2E4 MPa×μm³, 3E4 MPa×μm³, 4E4 MPa×μm³, 5E4 MPa×μm³, 6E4 MPa×μm³, 7E4 MPa×μm³, 8E4 MPa×μm³, 9E4 MPa×μm³, 1E5 MPa×μm³, 2E5 MPa×μm³, 3E5 MPa×μm³, 4E5 MPa×μm³, 5E5 MPa×μm³, 6E5 MPa×μm³, 7E5 MPa×μm³, 8E5 MPa×μm³, 9E5 MPa×μm³, 1E6 MPa×μm³, 2E6 MPa×μm³, 3E6 MPa×μm³, 4E6 MPa×μm³, 5E6 MPa×μm³, 6E6 MPa×μm³, 7E6 MPa×μm³, 8E7 MPa×μm³, 9E6 MPa×μm³, 1E7 MPa×μm³, or more. The optical portion may exhibit a maximum rigidity of at most about 1E7 MPa×μm³, 9E6 MPa×μm³, 8E6 MPa×μm³, 7E6 MPa×μm³, 6E6 MPa×μm³, 5E6 MPa×μm³, 4E6 MPa×μm³, 3E6 MPa×μm³, 2E6 MPa×μm³, 1E6 MPa×μm³, 9E5 MPa×μm³, 8E5 MPa×μm³, 7E5 MPa×μm³, 6E5 MPa×μm³, 5E5 MPa×μm³, 4E5 MPa×μm³, 3E5 MPa×μm³, 2E5 MPa×μm³, 1E5 MPa×μm³, 9E4 MPa×μm³, 8E4 MPa×μm³, 7E4 MPa×μm³, 6E4 MPa×μm³, 5E4 MPa×μm³, 4E4 MPa×μm³, 3E4 MPa×μm³, 2E4 MPa×μm³, 1E4 MPa×μm³, 9E3 MPa×μm³, 8E3 MPa×μm³, 7E3 MPa×μm³, 6E3 MPa×μm³, 5E3 MPa×μm³, 4E3 MPa×μm³, 3E3 MPa×μm³, 2E3 MPa×μm³, 1E3 MPa×μm³, or less. The optical portion may exhibit a maximum rigidity that is within a range defined by any two of the preceding values. The optical portion can exhibit a maximum rigidity, for example, within a range from 2E3 MPa×μm³ to 3E9 MPa×μm³, from 1E3 MPa×μm³ to 1E9 MPa×μm³, from 1E4 MPa×μm³ to 1E8 MPa×μm³, or from 1E5 MPa×μm³ to 1E7 MPa×μm³.

The dynamic contact lens can be configured to produce a tear volume that in conjunction with other optical elements can correct vision when applied to a cornea.

When a dynamic contact lens is applied to a cornea, the optical portion can assume two or more quasi-stable configurations, wherein the two or more quasi-stable configurations are characterized by a different gap between the center optical posterior surface and the anterior surface of the cornea. The dynamic contact lens can be configured such that the optical portion can transition between the two or more quasi-stable configurations by interaction with an eyelid and/or by eye movement such as by pressure applied to the dynamic contact lens by an eyelid and/or by a change in gaze angle.

The optical portion can have a diameter of at most about 10 mm, 9 mm, 8 mm, 7 mm, 6 mm, 5 mm, 4 mm, 3 mm, 2 mm, 1 mm, or less. The optical portion may have a diameter of at least about 1 mm, 2 mm, 3 mm, 4 mm, 5 mm, 6 mm, 7 mm, 8 mm, 9 mm, 10 mm, or more. The optical portion may have a diameter that is within a range defined by any two of the preceding values. The optical portion can have a diameter, for example, from 2.5 mm to 7 mm, from 2.5 mm to 6.5 mm, from 2.5 mm to 6.0 mm, from 2.5 mm to 5 mm, or from 2 mm to 4 mm.

The optical posterior surface may have a radius of curvature of at most about 10 mm, 9.5 mm, 9 mm, 8.5 mm, 8 mm, 7.5 mm, 7 mm, 6.5 mm, 6 mm, 5.5 mm, 5 mm, 4.5 mm, 4 mm, 3.5 mm, 3 mm, 2.5 mm, 2 mm, 1.5 mm, 1 mm, or less. The optical posterior surface may have a radius of curvature of at least about 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, or more. The optical posterior surface may have a radius of curvature that is within a range defined by any two of the preceding values. The optical posterior surface can have a radius of curvature, for example, from 3 mm to 7.5 mm, from 3 mm to 7 mm, from 3.5 mm to 6.5 mm, or from 4 mm to 6 mm.

The optical portion can have a substantially uniform thickness. The optical portion may comprise a substantially uniform thickness of at least about 10 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1,000 μm, or more. The optical portion may comprise a substantially uniform thickness of at most about 1,000 μm, 950 μm, 900 μm, 850 μm, 800 μm, 750 μm, 700 μm, 650 μm, 600 μm, 550 μm, 500 μm, 450 μm, 400 μm, 350 μm, 300 μm, 250 μm, 200 μm, 150 μm, 100 μm, 90 μm, 80 μm, 70 μm, 60 μm, 50 μm, 40 μm, 30 μm, 20 μm, 10 μm, or less. The optical portion may comprise a substantially uniform thickness that is within a range defined by any two of the preceding values. For example, the optical portion can have a substantially uniform thickness from 20 μm to 300 μm, from 20 μm to 250 μm, from 50 μm to 200 μm, or from 50 μm to 150 μm.

The optical portion can have a non-uniform thickness. The optical portion may comprise a non-uniform thickness, such as a center thickness, of at least about 10 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1,000 μm, or more. The optical portion may comprise a non-uniform thickness, such as a center thickness, of at most about 1,000 μm, 950 μm, 900 μm, 850 μm, 800 μm, 750 μm, 700 μm, 650 μm, 600 μm, 550 μm, 500 μm, 450 μm, 400 μm, 350 μm, 300 μm, 250 μm, 200 μm, 150 μm, 100 μm, 90 μm, 80 μm, 70 μm, 60 μm, 50 μm, 40 μm, 30 μm, 20 μm, 10 μm, or less. The optical portion may comprise a non-uniform thickness, such as a center thickness, that is within a range defined by any two of the preceding values. For example, an optical portion having a non-uniform thickness such as a center thickness from 20 μm to 300 μm, from 20 μm to 250 μm, from 50 μm to 200 μm, or from 50 μm to 150 μm, and the thickness of the optical portion can either increase or decrease from the center of the optical portion toward the interface with the peripheral portion. The thickness of the optical portion can vary throughout the cross-sectional profile of the optical portion. For example, the cross-sectional thickness of the optical portion can be different or the same at different radial distances from the center of the optical portion. For example, the thickness can be relatively high at the center, decrease away from the center, then increase, then decrease toward the interface with the peripheral portion. In general, to facilitate comfort it can be desirable that the anterior surface of the optical portion have a smooth profile, and therefore any thickness changes of the optical portion be applied to the posterior surface of the optical portion. A transition zone between the optical portion and the peripheral portion can be configured to facilitate the transition between quasi-stable configurations, and to maintain quasi-stable configurations.

The transition zone can be configured to facilitate flow of tear fluid to an optical tear volume formed between the optical posterior surface and the anterior surface of the cornea when the dynamic contact lens is applied to an eye.

For example, the transition zone can include channels or grooves that facilitate the ability of tear fluid to flow into and out of the optical tear volume defined by the optical portion.

The one or more grooves can be disposed in the posterior surface of the peripheral portion and can extend from the peripheral portion to the perimeter of optical portion. The one or more grooves can terminate at the transition zone or can extend across and transect the transition zone. The one or more grooves can extend into the optical portion.

For example, each of the one or more grooves can extend radially outward from the optical portion.

The one or more grooves may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more grooves. The one or more grooves may comprise at most about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 grooves. The one or more grooves may comprise a number of grooves that is within a range defined by any two of the preceding values. The one or more grooves can comprise, for example, from 1 to 20 grooves, from 1 to 16 grooves, from 1 to 12 grooves, from 4 to 10 grooves or from 4 to 8 grooves.

Each of the one or more grooves may comprise a width of at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. Each of the one or more grooves may comprise a width of at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. Each of the one or more grooves may comprise a width that is within a range defined by any two of the preceding values. Each of the one or more grooves can have a width, for example, from 30 μm to 1,000 μm, from 30 μm to 800 μm, from 30 μm to 600 μm, from 200 μm to 600 μm, or from 400 μm to 600 μm. It can be appreciated that each groove can have a varying width along the length from the distal end toward the perimeter of the contact lens to the proximate end toward the center of the dynamic contact lens.

Each of the one or more grooves may independently have a height or depth of at least about 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 1,000 μm, or more. Each of the one or more grooves may independently have a height or depth of at most about 1,000 μm, 950 μm, 900 μm, 850 μm, 800 μm, 750 μm, 700 μm, 650 μm, 600 μm, 550 μm, 500 μm, 450 μm, 400 μm, 350 μm, 300 μm, 250 μm, 200 μm, 150 μm, 100 μm, 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 10 μm, or less. Each of the one or more grooves may independently have a height or depth that is within a range defined by any two of the preceding values. Each of the one or more grooves can independently have a height/depth, for example, from 25 μm to 200 μm, from 25 μm to 150 μm, or from 100 μm to 200 μm.

Each of the one or more grooves may independently have a length of at least about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, or more. Each of the one or more grooves may independently have a length of at most about 10 mm, 9 mm, 8 mm, 7 mm, 6 mm, 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, or less. Each of the one or more grooves may independently have a length that is within a range defined by any two of the preceding values. Each of the one or more grooves can independently have a length, for example, from 0.5 mm to 7 mm, from 0.5 mm to 6 mm, from 0.5 mm to 5 mm, from 1 mm to 4 mm, or from 1 mm to 3 mm.

Each of the one or more grooves can independently have a cross-sectional profile and/or height/depth that is constant throughout the length of the groove.

Each of the one or more grooves can independently have a cross-sectional profile and/or height/depth that varies throughout the length of the groove. For example, the width of a groove can be wider toward the ends and narrower in the middle.

A groove or channel can have any suitable cross-sectional profile for facilitating the flow of tear fluid such as, for example, triangular, square, rectangular, dome-shaped, or oval.

At least one of the grooves can be coupled to one or more fenestrations extending through the peripheral anterior surface. A fenestration can be configured to fluidly couple a tear fluid layer or the anterior surface of the lens to a groove or to the tear film between the peripheral posterior surface of the lens and the cornea. For example, a groove can be coupled to one, two, three or more fenestrations.

Each of the one or more fenestrations may independently have a diameter of at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. Each of the one or more fenestrations may independently have a diameter of at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. Each of the one or more fenestrations may independently have a diameter that is within a range defined by any two of the preceding values. Each of the one or more fenestrations can independently have a diameter, for example, from 30 μm to 600 μm, from 100 μm to 500 μm. A fenestration can have any suitable cross-sectional profile to facilitate and/or control the flow of tear fluid across the surfaces of the dynamic contact lens.

The transition zone can comprise features configured to enhance the flexibility of the optical portion. Examples of features to enhance the flexibility of the optical portion, to facilitate the ability of the optical portion to transition between quasi-stable configurations, and/or to facilitate the ability of the optical portion to maintain a quasi-stable configuration include smooth edges, a thinned cross-sectional thickness, grooves, or a combination of any of the foregoing.

The transition zone can comprise one or more features or mechanisms that facilitates the exchange of tear fluid between the optical tear volume and a source of tear fluid external to the optical tear volume such as a tear reservoir or a tear meniscus.

For example, a dynamic contact lens provided by the present disclosure can comprise an optical portion having a diameter from 2.5 mm to 7 mm, a posterior optical posterior surface having a radius of curvature from 3 mm to 7.5 mm, a substantially uniform thickness with a center thickness from 20 μm to 300 μm, one or more grooves extending radially outward from the optical portion toward the peripheral edge of the lens, where the one or more grooves is from 3 to 20 grooves, where each groove has a width from 20 μm to 1,000 μm, a height/depth from 50 μm to 200 μm, a length from 1 mm to 7 mm, and one or more fenestrations coupled to each of the one or more grooves, where the fenestrations have a diameter from 100 μm to 600 μm.

As another example, a dynamic contact lens provided by the present disclosure can comprise an optical portion having a diameter from 2.5 mm to 7 mm, an optical posterior surface having a radius of curvature from 3 mm to 7.5 mm, a substantially uniform thickness with a center thickness from 50 μm to 300 μm, one or more grooves extending radially outward from the optical portion toward the peripheral edge of the lens, where the one or more grooves is from 1 to 10 grooves, where each groove has a width from 400 μm to 600 μm, a height/depth from 25 μm to 150 μm, a length from 1 mm to 5 mm, and one or more fenestrations coupled to each of the one or more grooves, where the fenestrations have a diameter from 300 μm to 500 μm.

Dynamic contact lenses provided by the present disclosure can comprise an optical portion, wherein the optical portion comprises a conforming configuration configured to provide a first optical power to an eye having a cornea; and at least one non-conforming configuration configured to provide a second optical power to the eye, wherein the second optical power is different than the first optical power; at least one first feature configured to induce a change between the conforming configuration and the at least one non-conforming configuration; and at least one second mechanism configured to induce a change between the at least one non-conforming configuration and the conforming configuration. The first and second mechanisms can be the same mechanisms or can be different mechanisms.

Dynamic contact lenses provided by the present disclosure can comprise an optical portion, wherein the optical portion comprises a first non-conforming configuration configured to provide a first optical power to an eye having a cornea; and at least one second non-conforming configuration configured to provide a second optical power to the eye, wherein the second optical power is different than the first optical power; at least one first feature configured to induce a change between the first non-conforming configuration and the at least one second non-conforming configuration; and at least one second mechanism configured to induce a change between the at least one non-conforming configuration and the conforming configuration. The first and second mechanisms can be the same mechanisms or can be different mechanisms.

When applied to an eye, the optical portion can assume a configuration in which the posterior surface of the optical portion conforms to or substantially conforms to the anterior surface of the cornea. It will be appreciated that in a conforming configuration, a thin tear film will be present between the posterior surface of the dynamic contact lens and the anterior surface of the cornea. The tear film may be at least about 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, of 10 μm thick, or more. The tear film may be at most about 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, or 0.1 μm thick, or less. The tear film may have a thickness that is within a range defined by any two of the preceding values. For example, the tear film can be from 0.1 μm to 3 μm thick, from 0.5 μm to 2.5 μm thick, or from 1 μm to 2 μm thick. A dynamic contact lens can be designed such that in a conforming configuration, the tear film thickness between the optical portion and the cornea can be greater than 3 μm and/or can vary across the optical portion to cause a change in shape of the optical anterior surface.

When applied to an eye, the optical portion can assume a first non-conforming configuration in which the posterior surface of the optical portion does not conform to the anterior surface of the cornea. For example, in a first non-conforming configuration the center gap between the anterior surface of the cornea and the posterior surface of the optical portion can at least about 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. In a first non-conforming configuration the center gap may be at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, or less. In a first non-conforming configuration the center gap may be within a range defined by any two of the preceding values. For instance, in a first non-conforming configuration the center gap may be greater than 3 μm, such as greater than 5 μm, greater than 10 μm, greater than 20 μm, greater than 30 μm, greater than 40 μm, greater than 50 μm, greater than 60 μm, greater than 70 μm, greater than 80 μm, or greater than 100 μm. For example, in a first non-conforming configuration the center gap between the anterior surface of the cornea and the posterior surface of the optical portion can be within a range from 5 μm to 100 μm, from 10 μm to 90 μm, from 10 μm to 70 μm, from 10 μm, to 50 μm, or from 10 μm to 30 μm. The optical portion can assume a second conforming configuration in which the center gap between the anterior surface of the cornea and the posterior surface of the optical portion which is greater than the center gap in the first non-conforming configuration, and can be within a range, for example, from 10 μm to 200 μm, or from 10 μm to 100 μm. It should be appreciated that the fundamental difference between the two configurations is that one configuration is more conforming to the cornea and the other configuration is less conforming and thus a change in the curvature of the optical anterior surface is created between the two non-conforming configurations, which provides a change in optical power when the optical portion is in either of the two quasi-stable non-conforming configurations.

The dynamic contact lens is fabricated such that the optical posterior curvature is different than the peripheral posterior curvature such that the gap height is small when no tear fluid flows under the optical portion and no mechanical force or fluid pressure is applied to the dynamic contact lens. However, when tear fluid flows under the optical portion such as induced by a gaze change, by eyelid pressure, and/or by connection of the any one of the lens features with a source of tear fluid, such as a tear meniscus, or other means that results in tear fluid flow into or out of the optical tear volume, the gap height changes, causing the dimensions of the optical tear volume to change and thereby change the optical power of the anterior surface of the optical portion. Tear fluid can be provided, for example, by a tear meniscus and/or by tear fluid reservoirs. The as-fabricated sagittal height can be designed based on the desired gap height and the desired change in optical power.

The gap height of the optical tear volume can assume from 10% to 100% of the as-fabricated sagittal height during gaze change, upon eyelid pressure and/or by connection of any one of the lens features with a source of tear fluid, such as the tear meniscus. The gap height can assume at least about 10%, 20%, 30%, 40%, 50%, 60%, 70%, 80%, 90%, or 100% of the as-fabricated sagittal height during gaze change. The gap height can assume at most about 100%, 90%, 80%, 70%, 60%, 50%, 40%, 30%, 20%, or 10% of the as-fabricated sagittal height during gaze change. The gap height can assume an amount of the as-fabricated sagittal height during gaze change that is within a range defined by any two of the preceding values. The percent the gap height can recover to that of the pre-fabricated sagittal height can at least in part be determined by the flow of tear fluid, the availability of tear fluid to flow under the optical portion, and structural features such as tear fluid reservoirs, grooves in the posterior surface of the lens, grooves in the anterior surface of the lens, fenestrations, transition zone geometry, peripheral and edge geometry, and/or other features such as material properties and surface properties that control and/or facilitate the flow of tear fluid under, over, and within the dynamic contact lens. In effect, the as-fabricated sagittal height as well as other structural features of the dynamic contact lens including, for example, the thickness, material modulus, rigidity, radius of curvature, and diameter contribute to imparting a restoring force to the optical portion in the anterior direction and away from the cornea that produces a pumping force to pull tear fluid beneath the optical portion to form a tear volume in at least one quasi-stable non-conforming configuration. This restoring force can be overcome by application of eyelid pressure or eye movement to the dynamic contact lens causing the optical portion to move in the posterior direction and toward the cornea to assume another quasi-stable non-conforming or conforming configuration.

In the conforming configuration the distance between the posterior surface of the optical portion and the cornea can be at least about 1 μm, 2 μm, 3 μm, 4 μm, 5 μm, 6 μm, 7 μm, 8 μm, 9 μm, 10 μm, or more. In the conforming configuration the distance between the posterior surface of the optical portion and the cornea can be at most about 10 μm, 9 μm, 8 μm, 7 μm, 6 μm, 5 μm, 4 μm, 3 μm, 2 μm, 1 μm, or less. In the conforming configuration the distance between the posterior surface of the optical portion and the cornea can be within a range defined by any two of the preceding values. For instance, in the conforming configuration the distance between the posterior surface of the optical portion and the cornea can be, for example, less than 3 μm, less than 2 μm, or less than 1 μm.

A dynamic contact lens can be fabricated such that the optical portion is designed to not conform to a cornea. In such embodiments the optical portion vaults over the cornea creating a gap height that is equal to or greater than 10 μm to create an optical tear volume that provides an optical power. For example, an optic zone of 3 mm in diameter with a base curve of 6.2 mm will create a gap height of 40 μm relative to the paracentral base curve; or, for example, an optic zone of 5 mm in diameter with a base curve (BC) of 6.4 mm will create a gap height of 100 μm relative to the peri-central BC. In the conforming configuration, the base curvature of the posterior surface of the optical portion can be substantially the same as the base curvature of the peripheral portion.

A dynamic contact lens can be designed such that the optical portion is made of a low-modulus material as disclosed herein, e.g., a material having a Young's modulus from 0.05 MPa to 10 MPa, or from 0.1 MPa to 2 MPa, such as to not conform to a cornea. In such embodiments, the optical portion vaults over the corneal curvature to create a gap that is equal to or greater than 10 μm to create an optical tear volume. For example, an optic zone of 3 mm in diameter with a BC of 6.2 mm will create a gap height of 40 μm relative to the pen-central BC; or, for example, an optic zone of 5 mm in diameter with a BC of 6.4 mm will create a gap height of 100 μm relative to the paracentral BC. As described herein, in a non-conforming configuration the gap height can be, for example, from 5 μm to 300 μm. In the conforming configuration the base curvature of the posterior surface of the optical portion can be substantially the same as the posterior base curvature of the peripheral portion.

A conforming configuration represents a quasi-stable state. By quasi-stable is meant that the configuration can be maintained for a period of time unless or until a force is applied or tear fluid becomes available to any of the lens features such that tear fluid can flow into or out of the optical tear volume to disrupt the quasi-stable equilibrium and cause a transition to another quasi-stable configuration.

The quasi-stable conforming configuration can be maintained by adhesion forces between the posterior surface of the optical portion and the anterior surface of the cornea. The quasi-stable conforming configuration can be maintained by mechanical and/or fluid dynamic forces of the dynamic contact lens. The quasi-stable conforming configuration can be maintained by a combination of adhesive forces, fluid dynamic, and lens mechanical forces.

The adhesion forces can be mediated by capillary forces that include, for example, the cohesive forces within the tear fluid and the adhesive forces between the film of tear fluid and the anterior surface of the cornea. The surface tension of the thin film of tear fluid between the posterior surface of the optical portion and the cornea can cause the two surfaces to adhere. With the tear fluid and the anterior ocular surface being hydrophilic, adhesive forces will be favored when the posterior surface of the optical portion is also hydrophilic. Conversely, when the dynamic posterior surface is hydrophobic the adhesive forces will be less.

Mechanical forces can arise from the selection of the thickness of certain regions of the lens, the selection of the curvature of certain regions of the lens, by the incorporation of features that facilitate manipulation of the lens by the eyelids and/or incorporation of features that facilitate fluid coupling and decoupling of the optical tear volume with a source of tear fluid such as a tear meniscus.

In the conforming configuration, the adhesive forces can extend across the entire optical posterior surface or can extend across a portion of the optical posterior surface.

In the conforming configuration, the gap between the optical posterior surface and the cornea can be substantially uniform across the diameter of the optical portion. A gap differential can be defined as a difference between the gap distance at the center of the optical portion and a gap distance at radial distances away from the center. In a conforming configuration, the gap differential is small and at a minimum. In the conforming configuration the gap differential is smaller than in a non-conforming configuration.

In the conforming configuration the optical portion can be configured to provide a first optical power to an eye. The first optical power may be zero (0D). The first optical power may be at least about 10 D, −9D, −8D, −7D, −6D, −5D, −4D, −3D, −2D, −1D, 0D, +1D, +2D, +3D, +4D, +5D, +6D, +7D, +8D, +9D, +10D, or more. The first optical power may be at most about +10D, +9D, +8D, +7D, +6D, +5D, +4D, +3D, +2D, +1D, 0D, −1D, −2D, −3D, −4D, −5D, −6D, −7D, −8D, −9D, −10D, or less. The first optical power may be within a range that is defined by any two of the preceding values. The first optical power can be within a range, for example, from 0D to ±10D, from 0D to ±8D, from 0D to ±6D, from 0D to ±4D, from 0D to ±3D, from 0D to ±2D, or from 0D to ±1D.

The dynamic optical portion can have one or more quasi-stable non-conforming configurations.

The one or more non-conforming configurations can comprise a single non-conforming configuration, two or more discrete non-conforming configurations, or a plurality of quasi-stable non-conforming configurations such as, for example, at least about 3, 4, 5, 6, 7, 8, 9, 10, or more quasi-stable configurations, which can be continuous or discrete. The one or more non-conforming configurations may comprise at most about 10, 9, 8, 7, 6, 5, 4, 3, or less quasi-stable configurations.

In the conforming configuration the gap differential between the posterior surface of the lens and the cornea at the center of the optical portion and the periphery of the optical portion toward the transition zone with the peripheral portion, is less in the conforming configuration than in a non-conforming configuration. In a conforming configuration the gap height may be at least about 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, or more. In a conforming configuration the gap height may be at most about 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, 0.1 μm, or less. In a conforming configuration the gap height may be within a range defined by any two of the preceding values. For example, in a conforming configuration the gap height can be from 0.1 μm to 4 μm, from 1 μm to 4 μm, or from 1 μm to 3 μm, such that in the conforming configuration the optical portion rests on the corneal tear film.

In a non-conforming configuration, the optical portion is not adhering to the cornea. The optical portion extends above or bulges away from the surface of the cornea to provide a lenticular volume between the posterior surface of the optical portion and the anterior surface of the cornea. The lenticular volume can fill with tear fluid to form an optical tear volume.

When on an eye, the non-conforming configuration of the dynamic contact lens in conjunction with the optical tear volume provides a second optical power to the eye, where the first optical power (in the conforming configuration) and the second optical power are not the same. The second optical power in a non-conforming configuration can be more or less than the optical power in the conforming configuration. The second optical power in a non-conforming configuration may be at least about −10D, −9D, −8D, −7D, −6D, −5D, −4D, −3D, −2D, −1D, −0.9D, −0.8D, −0.7D, −0.6D, −0.5D, −0.4D, −0.3D, −0.2D, −0.1D, 0D, +0.1D, +0.2D, +0.3D, +0.4D, +0.5D, +0.6D, +0.7D, +0.8D, +0.9D, +1D, +2D, +3D, +4D, +5D, +6D, +7D, +8D, +9D, +10D, or more. The second optical power in a non-conforming configuration may be at most about +10D, +9D, +8D, +7D, +6D, +5D, +4D, +3D, +2D, +1D, +0.9D, +0.8D, +0.7D, +0.6D, +0.5D, +0.4D, +0.3D, +0.2D, +0.1D, 0D, −0.1D, −0.2D, −0.3D, −0.4D, −0.5D, −0.6D, −0.7D, −0.8D, −0.9D, −1D, −2D, −3D, −4D, −5D, −6D, −7D, −8D, −9D, −10D, or less. The second optical power in a non-confirming configuration may be within a range defined by any two of the preceding values. For example, the second optical power can be less than ±1D, less than ±2D, less than ±3D, less than ±4D, less than ±5D, less than ±6D, less than ±7D, less than ±8D, less than ±9D, or less than ±10D of the first optical power. For example, the second optical power can be from 0.1D to 10D, from 0.1D to 9D, from 0.1D to 8D, from 0.1D to 7D, from 0.1D to 6D, from 0.1D to 5D, from 0.1D to 4D, from 0.1D to 3D, from 0.1D to 2D, or from 0.1D to 1D of the first optical power. For example, the second optical power can be from −0.1D to −10D, from −0.1D to −9D, from −0.1D to −8D, from −0.1D to −7D, from −0.1D to −6D, from −0.1D to −5D, from −0.1D to −4D, from −0.1D to −3D, from −0.1D to −2D, or from −0.1D to −1D of the first optical power.

In certain dynamic contact lenses, the first optical power does not provide a change in optical power to the eye; and in certain dynamic contact lenses the second optical power does not provide a change in optical power to the eye.

In certain dynamic contact lenses, the conforming configuration provides a first change in optical power to the eye; and the at least one non-conforming configuration provides a second change in optical power to the eye. It should be appreciated that the optical power of a dynamic contact lens is derived from the optical anterior surface. The optical power is related to effects of the optical tear volume on the optical anterior surface of the dynamic contact lens.

For a single non-conforming configuration, the optical portion can assume a single configuration in which the optical portion is not adhering to the cornea. The single non-conforming configuration can be quasi-stable. The single non-conforming configuration can have substantially the same shape as the as-fabricated optical portion.

A non-conforming configuration can comprise two or more discrete configurations. Each of the two or more discrete non-conforming configurations can impart a different optical power to the eye. The different optical powers are created by the anterior optical surface, the shape of which corresponds with the shape of the optical tear volume formed by between the optical posterior surface and the anterior surface of the cornea. Each of the two or more discrete configurations can be quasi-stable.

A non-conforming configuration can comprise a plurality of non-conforming configurations which can be discrete or continuous. These discrete or continuous non-conforming configurations can be quasi-stable or may not be stable. One or more of the plurality of discrete or continuous non-conforming configurations can be quasi-stable. For example, a quasi-stable configuration included within a plurality of continuous non-conforming configurations can comprise substantially the shape of the as-fabricated optical portion.

A non-conforming configuration can be characterized by a center gap height with respect to the base curvature of the peripheral portion. The posterior surface of the peripheral portion 106 can be characterized by a single curvature, which as shown in FIG. 1, can be extrapolated 119 to extend beneath the optical portion of the dynamic contact lens. In non-conforming configurations, the distance between the posterior surface of the optical portion and the peripheral base curvature is the gap height with respect to the peripheral base curvature. The gap height can decrease radially from the center of the optical portion toward the periphery of the optical portion in a non-conforming configuration.

In certain designs, the as-fabricated sagittal height and the gap height can increase and then decrease toward the transition zone between the optical portion with the peripheral portion.

The anterior surface of the lens can have a multifocal structure such that, for example, when the optical portion assumes a non-conforming configuration the optical portion provide additional optical power to the eye, and the region peripheral to the optical portion provides the same optical power as in the conforming configuration.

The entire dynamic lens configuration can be coupled with a multi-focal lens design to provide advantages of a multi-focal lens while also providing additional optical power from the dynamic lens under desired conditions, for example, for intermediate and near vision.

When placed on an eye, the peripheral portion can conform to the cornea and can rest on a tear film, and the peripheral base curvature can be substantially the same as the corneal curvature, and the gap height can be referenced with respect to the anterior surface of the cornea.

In a non-conforming configuration, the center gap height of the optical portion can be greater than the center gap height than in the conforming configuration.

In a non-conforming configuration, the gap height differential will be greater than the gap height differential in the conforming configuration.

A dynamic contact lens provided by the present disclosure can comprise one or more features configured to induce a change in configuration of the optical portion.

The one or more mechanisms or features can induce a change in configuration upon application of pressure to the feature by an eyelid or by contact with the tear meniscus. A mechanism for applying eyelid pressure can be passive, active, or a combination thereof. A passive mechanism can comprise, but does not require, a conscious action by the wearer of a dynamic contact lens. For example, a passive mechanism can comprise changing a gaze angle. An active mechanism can involve a conscious action by the dynamic contact lens wearer to induce a transition from one configuration to another. An example of an active mechanism includes consciously blinking or consciously squinting to induce a transition from one configuration of the optical portion to another configuration of the optical portion. A conscious mechanism can comprise repeated blinking or holding the eyelids closed for a period of time.

The mechanism for inducing a conformational change such as a change in quasi-stable configurations, can also comprise internal forces within the lens that can cause the optical portion to bulge once the capillary forces are overcome. For example, for a lens fabricated with a bulge, a bulging conformation can represent a low-energy configuration. After the capillary forces are reduced to release a conforming optical portion, the physical structure of the dynamic contact lens will act as a force to cause the optical portion to bulge away from the cornea and assume or approach as as-fabricated shape. The mechanism for inducing a transition between conforming and non-conforming states may not involve capillary forces. Mechanical forces within the lens can cause the optical portion to transition between configurations. Tear fluid can flow into the volume between the posterior surface of the dynamic contact lens and the cornea to form an optical tear volume during or after the optical portion has transitioned between configurations such as from a conforming configuration to a non-conforming configuration. The mechanical forces and/or fluid dynamic forces can arise from the selection of the design of the dynamic contact lens and the selection of the materials forming different parts of the lens. For example, design elements include the thickness, the rigidity, and/or the radius of curvature of different portions of the as-fabricated dynamic contact lens and including the disposition of protrusions on the anterior surface of the dynamic contact lens. Examples of material properties include the modulus, hydrophobicity, and/or hydrophilicity of the materials forming different portions of the dynamic contact lens and the rigidities and/or relative rigidities of different portions of the optical portion, the transition zone, and the peripheral portion of the dynamic contact lens.

The at least one first mechanism and the at least one second mechanism can be the same mechanism or can be different mechanisms including, for example, capillary forces and/or internal mechanical forces.

A dynamic contact lens provided by the present disclosure can comprises a center geometric axis.

The optical portion can be disposed at the center of the geometric axis, para-central to the center geometric axis, off the center of the geometric axis, or a combination of any of the foregoing. For example, the optical portion can be centrosymmetric and be centered at the geometric axis of the dynamic contact lens. A para-central optical portion can be symmetrically disposed at a radial distance about the center geometric axis of the dynamic contact lens. An optical portion can also be located away from the center of the geometric axis.

In the conforming configuration, the optical posterior surface can be configured to substantially conform to the anterior surface of the cornea.

In the conforming configuration, the optical portion can be configured to adhere to the cornea. By adhesion to the cornea is meant that in a conforming configuration the optical portion will assume a quasi-stable configuration in which the posterior surface of the optical portion is separated from the anterior surface of the cornea by a thin layer of tear fluid. The adhesion to the cornea can be temporary. The adhesion can be such as to establish a quasi-stable equilibrium. The quasi-stable equilibrium can be disrupted by application of a force such as mechanical force and/or a fluid dynamic force.

The optical portion can adhere to the corneal surface by capillary forces.

A layer of liquid between two wetted surfaces can be referred to as a capillary bridge. A capillary adhesive force between the two surfaces is caused by capillary action pulling the liquid outward from the narrow gap. The capillary adhesive force pulling the two surfaces toward each other can maintain the relative position of the two surfaces in an equilibrium state. Disrupting the equilibrium such as, for example, by forcing the opposing surfaces apart can reduce the capillary adhesive forces and cause the surfaces to separate.

In a non-conforming configuration, a tear volume can be formed within the optical tear volume between the posterior surface of the optical portion and the surface of the cornea. The tear fluid for filling the tear volume can originate, for example, from tear fluid reservoirs, from the tear film between the dynamic contact lens such at the peripheral portion of the dynamic contact lens, from the periphery of the dynamic contact lens such as proximate to the conjunctiva, from the tear meniscus, through fenestrations spanning the thickness of the dynamic lens, through grooves in the posterior surface and/or the anterior surface of the dynamic contact lens, or a combination of any of the foregoing. In dynamic contact lenses comprising fenestrations extending from the anterior surface of the dynamic contact lens to the posterior surface or into posterior grooves, tear fluid can also originate from tear fluid on the anterior surface of the dynamic contact lens and/or from the tear meniscus of the eye.

The optical portion of the dynamic contact lens can be configured to provide a different optical power for at least two different depths of vision. The depths of vision can include, for example, near vision, intermediate vision, and distance vision.

For example, a dynamic contact lens can be configured such that when applied to the cornea, the optical portion provides a corrected first vision in the conforming configuration and provides a corrected second vision in the at least one non-conforming configuration.

For example, a dynamic contact lens can be configured such that when applied to the cornea, the optical portion provides an uncorrected first vision in the conforming configuration and provides a corrected second vision in the at least one non-conforming configuration.

For example, a dynamic contact lens can be configured such that when applied to the cornea, the optical portion provides a corrected first vision in the conforming configuration and provides an uncorrected second vision in the at least one non-conforming configuration.

Each of the first vision and the second vision can independently comprise a distance vision, an intermediate vision, or a near vision. For example, a dynamic contact lens can be configured such that when applied to the cornea, the optical portion provides an uncorrected first vision in the conforming configuration and provides a corrected second vision in the at least one non-conforming configuration.

Dynamic contact lenses provided by the present disclosure can facilitate the exchange of tear fluid between the optical tear volume beneath the optical portion with tear fluid overlying the peripheral anterior surface of the dynamic contact lens such as tear fluid on the peripheral anterior surface and/or the tear meniscus, upon interaction of the dynamic contact lens with motion of the eyelids or eye movement such as a change in gaze angle. The inner optical portion of the contact lens is dynamic such that the optical portion can assume at least two quasi-stable configurations when worn on the eye of a patient. The posterior surface of the optical portion and the anterior surface of the cornea define a dynamic optical tear volume such that the optical tear volume is different in the two quasi-stable configurations. The optical tear volume can change the shape of the optical anterior surface to change the optical power of the optical portion.

The dynamic contact lens is configured to facilitate the ability of the optical portion to change configuration as a wearer changes vision, such as from near to far vision, or from far to near vision. To accommodate the need to continuously change the optical power of the optical portion the optical tear volume must change rapidly. For example, a transition between quasi-stable configurations can be less than 3 seconds, less than 2 seconds, or less than 1 seconds to accommodate changes in a patient's vision. The dynamic contact lens must therefore be continuously and repeatedly responsive to a user's vision.

When a dynamic contact lens is applied to an eye, there is a tear film between the peripheral posterior surface of the contact lens and the cornea. For a peripheral posterior surface that conforms to the anterior surface of the cornea, the tear film generally has a thickness of from 0.1 μm to 3 μm and for an area of about 0.005 mm³ to about 0.15 mm³ under a typical of a 14 mm-diameter contact lens, has a total volume of about 0.005 μL to 0.15 μL.

Dynamic contact lenses can be configured to have a maximum optical tear volume, such as a maximum optical tear volume of at least about 0.01 μL, 0.002 μL, 0.003 μL, 0.004 μL, 0.005 μL, 0.006 μL, 0.007 μL, 0.008 μL, 0.009 μL, 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, or more. The dynamic contact lenses may be configured to have a maximum optical tear volume of at most about 1 μL, 0.9 μL, 0.8 μL, 0.7 μL, 0.6 μL, 0.5 μL, 0.4 μL, 0.3 μL, 0.2 μL, 0.1 μL, 0.09 μL, 0.08 μL, 0.07 μL, 0.06 μL, 0.05 μL, 0.04 μL, 0.03 μL, 0.02 μL, 0.01 μL, 0.009 μL, 0.008 μL, 0.007 μL, 0.006 μL, 0.005 μL, 0.004 μL, 0.003 μL, 0.002 μL, 0.001 μL, or less. The dynamic contact lenses may be configured to have a maximum optical tear volume that is within a range defined by any two of the preceding values, such as, for example, from 0.01 μL to 1 μL such as from 0.05 μL to 0.8 μL, from 0.1 μL to 0.7 μL, from 0.2 μL to 0.6 μL. Tear fluid distribute non-uniformly over the eye surface in the different compartments, such as the surface tear film, the upper and lower menisci, and in the cul-de-sac (under the eyelid).

The tear fluid in the tear film under the contact lens is very shallow (up to 0.15 μL) and does not have the capacity to fill the tear volume between the posterior surface of the optical portion and the cornea in a dynamic contact lens, the lower and upper tear menisci have sufficient tear fluid, from about 1.5 μL to about 3 μL to provide fluid for the tear volume.

Dynamic contact lenses can be configured to have a maximum optical tear volume, for example, from 0.01 μL to 1 μL such as from 0.05 μL to 0.8 μL, from 0.1 μL to 0.7 μL, from 0.2 μL to 0.6 μL.

Dynamic contact lenses provided by the present disclosure can comprise one or more mechanisms for facilitating the transition between the two or more quasi-stable configurations and for maintaining the two or more quasi-stable configurations. The mechanisms are configured to facilitate and to control the flow of tear fluid into and out of the optical tear volume between the optical portion of the contact lens and the cornea. This volume is referred to as the optical tear fluid volume and is distinct from other tear volumes such as tear fluid reservoirs. These transition mechanisms can operate independently or in conjunction with any mechanical mechanisms incorporated into the dynamic contact lens.

The two quasi-stable configurations of the optical portion the optical tear volume is different. During transitions between the two quasi-stable configurations tear fluid must be transported out of or into the optical tear volume. Accordingly, when tear fluid is expelled from the optical tear volume there must be somewhere for the tear fluid to flow. The tear fluid can flow into the tear film along the interface between the posterior surface of the contact lens and the cornea toward the perimeter of the lens. Also, features can be incorporated into the contact lens to facilitate the ability of tear fluid from the optical portion to flow to the anterior surface of a contact lens and/or into a groove or cavity incorporated into the posterior surface and/or the anterior surface of the peripheral portion of the contact lens. Conversely, when tear fluid flows into the optical tear volume as the optical portion transitions from one quasi-stable configuration to another, there must be a source of tear fluid to draw from. The source of the tear fluid can be the tear film between the posterior surface of the contact lens and the cornea. The source of tear fluid can be the anterior surface of the contact lens, or a feature such as a groove or cavity incorporated into the posterior and/or anterior surface of the peripheral portion that is filled with tear fluid. The source can also be the tear fluid present in the tear meniscus area. Thus, the mechanism configured to facilitate and to control the flow of tear fluid can also serve as a source of tear fluid that can be exchanged with the tear fluid in the optical tear volume during transitions between the quasi-stable configurations. Other features and mechanisms such as a combination of fenestrations and grooves can serve to fluidly couple the tear meniscus at the perimeter of the eye to the optical tear volume.

Examples of mechanisms for facilitating and controlling the flow of tear fluid into and out of the optical tear fluid volume include posterior grooves, fenestrations, tear fluid reservoirs, cavities, indentations, protrusions, anterior grooves, valves, and combinations of any of the foregoing.

A mechanism can comprise one or more grooves disposed in the anterior and/or posterior surface of a dynamic contact lens.

A groove can be configured to transport tear fluid into and out of the optical tear volume.

A groove can be configured to transport tear fluid from toward the perimeter of the contact lens to the optical tear volume, and from the optical tear volume toward the perimeter of the contact lens.

A groove can be configured to transport tear fluid into and out of a tear fluid reservoir.

A groove can be configured to transport tear fluid from toward the perimeter of the contact lens into a tear fluid reservoir and from a tear fluid reservoir toward the perimeter of the contact lens.

A groove can be configured to transport tear fluid from a tear fluid reservoir into the and out of the optical tear volume.

A groove can be configured to transport fluid between tear fluid reservoirs.

A groove can be configured to transport tear fluid and to serve as a tear fluid reservoir.

A groove can be non-compressible, compressible, or partially compressible by force applied by an eyelid.

A groove can be fluidly coupled to the optical tear volume, fluidly coupled to the tear meniscus, fluidly coupled to a tear fluid reservoir, or a combination of any of the foregoing.

A groove may not be coupled the optical tear volume, fluidly coupled to the tear meniscus, fluidly coupled to a tear fluid reservoir, or a combination of any of the foregoing.

A groove may have any suitable cross-sectional profile such as a truncated round, oval, square, rectangular or triangular cross-sectional profile.

The cross-sectional profile and dimensions of a groove may be substantially throughout the length of the groove. The cross-sectional profile and/or dimensions may vary throughout the length of the groove. For example, the width of a groove and/or the depth of a groove can change throughout the length of the groove or in different portions along the length of the groove.

The width of a groove may be at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The width of a groove may be at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. The width of a groove may be within a range defined by any two of the preceding values. The width of a groove can be, for example, from 20 μm to 1,000 μm, from 20 μm to 800 μm, from 20 μm to 600 μm, from 200 μm to 600 μm, or from 400 μm to 600 μm.

The height of a groove may be at least about 10 μm, 20 μm, 30 μm, 40 μm, 50 μm, 60 μm, 70 μm, 80 μm, 90 μm, 100 μm, 150 μm, 200 μm, 250 μm, or more. The height of a groove may be at most about 250 μm, 200 μm, 150 μm, 100 μm, 90 μm, 80 μm, 70 μm, 60 μm, 50 μm, 40 μm, 30 μm, 20 μm, 10 μm, or less. The height or depth of a groove can be, for example, from 20 μm to 200 μm, from 20 μm to 150 μm, or from 100 μm to 200 μm.

A groove may have a length of at least about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, or more. A groove may have a length of at most about 10 mm, 9 mm, 8 mm, 7 mm, 6 mm, 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, or less. A groove may have a length that is within a range defined by any two of the preceding values. A groove can have a length, for example, from 0.5 mm to 7 mm, from 1 mm to 6 mm, from 1 mm to 5 mm, from 1 mm to 4 mm, or from 1 mm to 3 mm.

The cross-sectional profile and dimensions of a groove can be different at different locations along the length of the groove. For example, a groove can have larger dimensions at an interface with the optical tear volume, the perimeter of the contact lens, or a tear fluid volume.

The surfaces of a groove can include features and/or a surface treatment for controlling the flow of tear fluid within the groove. The features can control the directional flow of tear fluid within the groove. Examples of suitable features include surface roughness, hydrophobic coatings, and hydrophilic coatings.

A groove can be fluidly coupled to one or more fenestrations. A fenestration can intersect a groove at any suitable location along the length of the groove. The fenestration can be configured to transport tear fluid to and from the groove to the anterior surface of the contact lens.

A dynamic contact lens can include a plurality of grooves, which can be symmetrically or asymmetrically disposed around the optical portion. A dynamic contact lens may include at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more grooves. A dynamic contact lens may comprise at most about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 grooves. A dynamic contact lines may comprise a number of grooves that is within a range defined by any two of the preceding values. For example, a dynamic contact lens can include, for example, from 1 to 40 grooves, from 1 to 30 grooves, from 1 to 20 grooves, from 2 to 15 grooves, from 3 to 10 grooves, from 4 to 8 grooves, or from 4 to 6 grooves.

The groove can be fluidly coupled to the optical tear volume such that one end of the groove intersects with the optical portion and the other end of the groove can be terminated at a radial distance from the center axis in the peripheral portion. This terminal groove end can be located at any suitable distance from the lens center and can extend to the perimeter of the dynamic contact lens. Each of the plurality of grooves can independently terminate at the same or different distance from the lens center.

The grooves can have any suitable orientation with respect to the optical portion and the central axis of the dynamic contact lens. A groove can be directed toward the lens center such that a plurality of grooves radiates from the lens center and the grooves orthogonally intersect the optical portion. For example, the grooves can be oriented in a spoke/hub configuration where the hub is in effect the optical portion of the contact lens. The plurality of grooves may not be directed toward the lens center and may non-orthogonally intersect the optical portion.

A groove can be configured to be compressible. A compressible or a groove can compress upon interaction with an eyelid or with motion of an eyelid. A groove can have a depth or height such that a portion of the contact lens overlying the groove is thin and is deformable by a force applied by an eyelid. The ability to deform can be facilitated by one or more additional mechanisms such as a protrusion on the anterior surface of the peripheral portion in proximity to the groove that facilitates the ability of an eyelid to interact with the contact lens. For example, interaction with motion of an eyelid or eye movement can apply downward pressure on the groove which is amplified by a proximal protrusion.

A groove can be coupled to a passive or active mechanism configured to rotate the dynamic contact lens to a certain angular position with respect to the optical axis of the eye to facilitate exchange of tear fluid between the optical tear volume and tear fluid external to the optical tear volume. For example, a dynamic contact lens can include one or mechanisms that facilitate the ability of the dynamic contact lens to rotate to fluidly couple or enhance the fluid coupling between the groove and the tear meniscus.

One or more grooves can be disposed in the anterior surface of the peripheral portion of the dynamic contact lens. An anterior groove can be fluidly coupled to the tear meniscus, to a fenestration, to a fluid reservoir, to a posterior groove, or a combination of any of the foregoing. An anterior groove can serve as a tear fluid reservoir. An anterior groove can be connected to one or more other anterior grooves.

One or more anterior and/or posterior grooves can be connected to a single fenestration and/or to a single groove or can be connected to multiple fenestrations and/or multiple grooves.

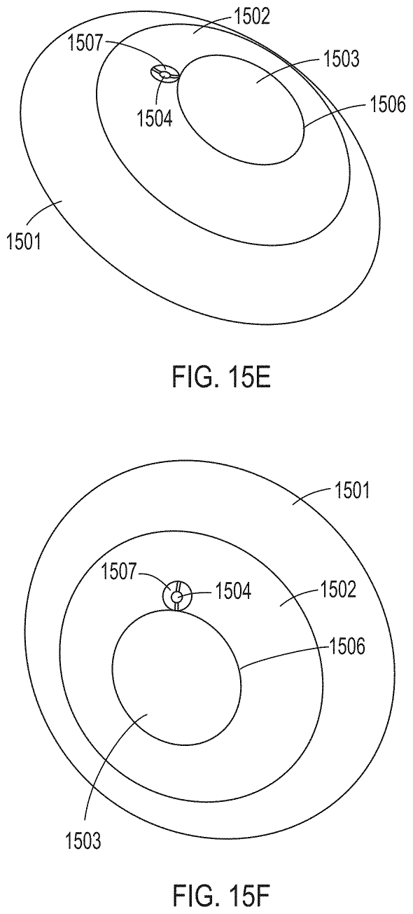

Two or more posterior and/or anterior grooves can be fluidly coupled. The fluidly coupled posterior and/or anterior grooves can be configured to facilitate fluid flow and/or to filter the tear fluid. The coupling can be such that over a certain distance the two or more grooves can overlap. By overlapping an anterior groove and a posterior groove the anterior and posterior surfaces can be fluidly coupled without fenestrations as shown in FIGS. 18A-18C.

A posterior groove and/or anterior groove can include wide sections that retain tear fluid and can serve s tear fluid reservoirs.

The edges of a groove can be chamfered to improve the flow of tear fluid and/or to enhance patient comfort. Chamfered edges can mitigate irritation caused by interaction of an eyelid, the conjunctiva, and/or the cornea with a groove.

A posterior and/or anterior groove can be disposed such that the groove is oriented in a desired position with respect to the eye. For example, a groove can be oriented toward the lower tear meniscus, toward the upper tear meniscus, or away from either tear meniscus. To facilitate orientation of a groove with respect to the eye, the dynamic contact lens can comprise one or more thickened or ballasted regions.

A cavity on the anterior side of the lens can also be a result of a recess on the posterior side of the lens and collapsing of the recess while on the eye.

A dynamic contact lens can comprise one or more fenestrations. Fenestrations can be configured to facilitate the transport of tear fluid to and from the anterior surface of a dynamic contact lens to the tear film and/or to a transition control mechanism such as a groove, a cavity, or a tear fluid reservoir.

A fenestration can be disposed in the peripheral portion of the lens such that the fenestration does not interfere with vision.

A fenestration can extend through the thickness of the peripheral portion thereby fluidly coupling tear fluid on the peripheral anterior surface with tear fluid on the peripheral posterior surface

A fenestration can be oriented substantially orthogonally to the anterior and posterior surfaces of the peripheral portion. A fenestration can be oriented at an angle with respect to the anterior and posterior surfaces of the peripheral portion. An angled orientation can facilitate directional control of tear fluid flow.

A fenestration can be fluidly coupled to a groove such as at the terminal end of a groove or at any place along the length of a groove.

A fenestration can have any suitable cross-sectional profile. For example, the cross-sectional profile of a fenestration can be round, oval, oblong, square, rectangular, or triangular.

A fenestration can have any suitable cross-sectional dimension. For example, a cross-sectional dimension of a fenestration can be from 20 μm to 600 μm, from 50 μm to 400 μm, or from 100 μm to 300 μm.

A fenestration can have a constant cross-sectional profile and dimension throughout the length, or a fenestration can have a cross-sectional profile that is different at different sections along the length. For example, a fenestration can have larger dimensions at the ends where the fenestration intersects with the anterior and/or posterior surfaces of the peripheral portion.

A fenestration can comprise a slit such as a cut extending through the thickness of the peripheral portion of the dynamic contact lens. A slit may have a length of at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, 1,500 μm, 2,000 μm, 2,500 μm, 3,000 μm, 3,500 μm, 4,000 μm, 4,500 μm, 5,000 μm, or more. A slit may have a length of at most about 5.000 μm, 4,500 μm, 4,000 μm, 3,500 μm, 3,000 μm, 2,500 μm, 2,000 μm, 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. A slit may have a length that is within a range defined by any two of the preceding values. A slit can have a length, for example, from 25 μm to 2,000 μm, from 50 μm to 1,500 μm, from 100 μm to 1,000 μm, or from 200 μm to 600 μm. A fenestration in the form of a slit can be an arc at a radial distance from the center axis of the lens or from the center axis of the optical portion. A slit can be oriented toward the center axis of the lens or from the center axis of the optical portion or ant an angle with respect to the center axis of the lens or from the center axis of the optical portion. A slit can be coupled to a groove and/or to a tear fluid reservoir or directly to the tear meniscus.

Terminal portions of a fenestration can include features that facilitate the ability of the fenestration to interact with pressure applied by an eyelid. Examples of such features include protrusions in proximity to the fenestration. For example, the protrusion or protrusions can be annular, can be disposed toward the perimeter of the contact lens, or can be disposed toward the optical portion.

Terminal portions of a fenestration can include features that facilitate the ability of a fenestration to transport tear fluid. For example, one or more cavities over the anterior surface of the lens can be situated in proximity to a fenestration that are configured to collect and retain tear fluid. For example, a fenestration can intersect the anterior surface of the peripheral portion at a cavity or depression that can fill with tear fluid. The cavity can be an annular depression surrounding the fenestration.

A fenestration can extend from the peripheral anterior surface to the posterior surface at the interface between the optical portion and the peripheral portion or to the posterior surface of the optical portion. A fenestration with this configuration can provide for tear fluid transport directly between the optical tear volume and the anterior surface of the contact lens.

A fenestration can be disposed in the optical portion. A fenestration disposed in the optical portion can provide for tear fluid transport directly between the optical tear volume and the anterior surface of the dynamic contact lens.

A fenestration can be configured to function as a valve.

A fenestration can be configured to function as a capillary valve.

A fenestration can have areas of elevation in proximity to the anterior orifice of the fenestration. The elevated areas, such as an elevated annular ring surrounding the anterior orifice can serve to limit tear fluid flow when the volume of tear fluid is below a certain amount. The area may be elevated above the anterior surface of the dynamic contact lens by at least about 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The area may be elevated above the anterior surface of the dynamic contact lens by at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, or less. The area may be elevated above the anterior surface of the dynamic contact lens by a distance that is within a range defined by any two of the preceding values. The area can be elevated above the anterior surface of the dynamic contact lens, for example, from 1 μm to 400 μm, from 5 μm to 300 μm, from 10 μm to 200 μm, or from 20 μm to 100 μm. The elevated area can be circumferential or partially circumferential about the anterior orifice of the fenestration. The elevated are can have different elevations or a gradient of elevation in different parts of the elevated area. The elevated area may be smoothened to minimize the interaction with the eyelid.

A mechanism for facilitating and controlling the flow of tear fluid can include one or more tear fluid reservoirs. A tear fluid reservoir is referred to as a cavity disposed in the posterior surface of the contact lens that is configured to provide a source of tear fluid and/or a volume for tear fluid to flow into. Tear fluid reservoirs are distinguished from cavities, which can be disposed in the anterior surface of the dynamic contact lens.

The reservoirs can be disposed in the in the peripheral posterior surface of a dynamic contact lens. When worn on the eye of a patient, the reservoirs can fill with tear fluid. When fluidly coupled to the optical tear volume, the reservoirs can serve as a source of tear fluid. A tear fluid reservoir can serve as a source of tear fluid for filling the optical tear volume when the optical portion assumes a first quasi-stable optical configuration and can serve as a receptacle to receive and retain tear fluid when the optical portion assumes a second quasi-stable configuration.

A reservoir can be configured to operate in a reciprocal manner with the optical portion to provide a pumping and pulling action in which tear fluid is alternately exchanged between the optical tear volume and a tear fluid reservoir.

A reservoir can be configured to provide a compressible tear fluid reservoir. For example, interaction of the dynamic contact lens with an eyelid can cause the reservoir to change conformation. During the change in conformation the reservoir can either expel tear fluid or can draw tear fluid into the reservoir. For example, the tear fluid reservoir can be fluidly coupled to the optical tear volume and can exchange tear fluid with the optical tear volume through a pumping and pulling action.

A compressible reservoir can have dimensions such as width and height that render the peripheral portion overlying the reservoir to be flexible.

A reservoir can have a height/depth of at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 2 mm, 3 mm, 4 mm, 5 mm, 6 mm, 7 mm, 8 mm, 9 mm, 10 mm, or more. A reservoir can have a height/depth of at most about 10 mm, 9 mm, 8 mm, 7 mm, 6 mm, 5 mm, 4 mm, 3 mm, 2 mm, 1 mm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. A reservoir can have a height/depth that is within a range defined by any two of the preceding values. A reservoir can have a height/depth, for example, from 10 μm to 800 μm, from 20 μm to 600 μm, from 50 μm to 500 μm, or from 100 μm to 400 μm. A reservoir can have a width/length, for example, from 50 μm to 5 mm, from 100 μm to 4 mm, from 200 μm to 3 mm, or from 500 μm to 2 mm.

A reservoir can be disposed at a radial distance from the center axis of the dynamic contact lens or from the center axis of the optical portion and can be in the shape of an arc or can extend circumferentially around the dynamic contact lens at a radial distance from the axis.

The thickness of the peripheral portion overlying a reservoir can be configured to deform. The thickness of the peripheral portion overlaying a reservoir may be at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The thickness of the peripheral portion overlaying a reservoir may be at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. The thickness of the peripheral portion overlaying a reservoir may be within a range defined by any two of the preceding values. For example, the thickness of the peripheral portion overlying a reservoir can be from 10 μm to 500 μm, from 10 μm to 400 μm, from 50 μm to 300 μm, or from 100 μm to 250 μm.

A reservoir can be any suitable shape. The shape can be symmetric or non-symmetric.

The shape can be oriented with respect to the optical portion. By oriented is meant that the reservoir can have a shape that is associated with the optical portion. For example, the reservoir can be narrower or wider toward the optical portion, or the reservoir can be radially symmetric with respect to the optical portion. For example, the reservoir can be round, oval, oblong, elongated in a radial dimension with respect to the center axis of the lens, or elongated in a centrosymmetric dimension.

A reservoir and the resulting tear fluid reservoir can be fluidly coupled to a groove, the optical tear volume, to another reservoir, to the tear meniscus, to a fenestration, or a combination of any of the foregoing.

A dynamic contact lens can include one or more reservoirs. The one or more reservoirs can be located in the posterior surface of the peripheral portion. The one or more cavities can be disposed symmetrically or non-symmetrically about the optical portion. The one or more reservoirs can be disposed at a radial distance from the center axis of the lens. The one or more reservoirs may be disposed at a radial distance of at least about 1 mm, 2 mm, 3 mm, 4 mm, 5 mm, 6 mm, 7 mm, 8 mm, 9 mm, 10 mm, or more. The one or more reservoirs may be disposed at a radial distance of at most about 10 mm, 9 mm, 8 mm, 7 mm, 6 mm, 5 mm, 4 mm, 3 mm, 2 mm, 1 mm, or less. The one or more reservoirs may be disposed at a radial distance that is within a range defined by any two of the preceding values. For example, a reservoir can be disposed at a radial distance from 2 mm to 7 mm, from 3 mm to 6 mm, or from 3 mm to 5 mm, from the lens center or from the center of the optical portion.

A reservoir may provide a volume of at least about 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 1.25 μL, 1.5 μL, 1.75 μL, 2 μL, 2.5 μL, 3 μL, 4 μL, 5 μL, or more. A reservoir may provide a volume of at most about 5 μL, 4 μL, 3 μL, 2.5 μL, 2 μL, 1.75 μL, 1.5 μL, 1.25 μL, 1 μL, 0.9 μL, 0.8 μL, 0.7 μL, 0.6 μL, 0.5 μL, 0.4 μL, 0.3 μL, 0.2 μL, 0.1 μL, 0.09 μL, 0.08 μL, 0.07 μL, 0.06 μL, 0.05 μL, 0.04 μL, 0.03 μL, 0.02 μL, 0.01 μL, or less. A reservoir may provide a volume that is within a range defined by any two of the preceding values. A reservoir can provide a volume from 0.05 μL up to 2 μL, from 0.1 μL to 1.5 μL, from 0.2 1.25 μL, or from 0.5 μL to 1 μL between the posterior surface of the peripheral portion and the cornea.

A reservoir can be associated with one or more mechanisms configured to facilitate the interaction of the tear fluid reservoir with pressure exerted by an eyelid and/or by movement of the eye. For example, one or more protrusions can be disposed on the anterior surface of the peripheral portion in proximity to the reservoir such that the one or more protrusions functions to amplify and/or direct a downward force exerted by an eyelid. The downward force on the reservoir can function to expel tear fluid from the reservoir and into the optical tear volume.

A mechanism for facilitating and controlling the flow of tear fluid can include one or more tear fluid depressions. A tear fluid depression can be disposed in the anterior surface of the contact lens that can be configured to provide a source of tear fluid and/or a volume for tear fluid to flow into. Tear fluid depressions are distinguished from reservoirs, which can be disposed in the posterior surface of the dynamic contact lens.

A depression can be disposed in the in the peripheral anterior surface of a dynamic contact lens. When worn on the eye of a patient, the cavities can fill with tear fluid. When fluidly coupled to the optical tear volume, the depressions can serve as a source of tear fluid. A tear fluid depression can serve as a source of tear fluid for filling the optical tear volume when the optical portion assumes a first quasi-stable optical configuration and can serve as a receptacle to receive and retain tear fluid when the optical portion assumes a second quasi-stable configuration.

A depression can be configured to operate in a reciprocal manner with the optical portion to provide a pumping and pulling action in which tear fluid is alternately exchanged between the optical tear volume and a cavity.

A depression can be disposed at a radial distance from the center axis of the dynamic contact lens or from the center axis of the optical portion and can be in the shape of an arc or can extend circumferentially around the dynamic contact lens at a radial distance from the axis.

A depression can be fluidly coupled to a groove, the optical tear volume, to another cavity, to the tear meniscus, to a fenestration, or a combination of any of the foregoing.

A dynamic contact lens can include one or more depressions. The one or more depressions can be located in the anterior surface of the peripheral portion. The one or more depressions can be disposed symmetrically or non-symmetrically about the optical portion. The one or more depression can be disposed at a radial distance from the center axis of the lens. A cavity may be disposed at a radial distance of at least about 1 mm, 2 mm, 3 mm, 4 mm, 5 mm, 6 mm, 7 mm, 8 mm, 9 mm, 10 mm, or more from the center of the optical portion. A cavity may be disposed at a radial distance of at most about 10 mm, 9 mm, 8 mm, 7 mm, 6 mm, 5 mm, 4 mm, 3 mm, 2 mm, 1 mm, or less from the center of the optical portion. A cavity may be disposed at a distance from the center of the optical portion that is within a range defined by any two of the preceding values. For example, a cavity can be disposed at a radial distance from 2 mm to 7 mm, from 3 mm to 6 mm, or from 3 mm to 5 mm, from the lens center or from the center of the optical portion.

A depression can be configured to fluidly couple to a tear meniscus. The tear meniscus has a height of about 200 μm to 300 μm at the above eyelid margins. Fenestrations having diameters from 25 μm to 500 μm are relatively small and can be difficult to couple to the shallow tear meniscus. To facilitate the ability of fenestrations to fluidly couple with the tear meniscus, the anterior orifice of a fenestration can be disposed within a depression or cavity in the anterior surface of the dynamic contact lens. A large depression, compared to the diameter of a fenestration, can facilitate the ability of the anterior orifice of the fenestration to fluidly couple to the tear meniscus. The depression may have a diameter of at least about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, or more. The depression may have a diameter of at most about 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, or less. The depression may have a diameter that is within a range defined by any two of the preceding values. The depression can have a diameter, for example, from 0.5 mm to 4, such as from 1 mm to 3 mm. A depression may have a depth of at least about 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 150 μm, 200 μm, 250 μm, or more. A depression may have a depth of at most about 250 μm, 200 μm, 150 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, or less. A depression may have a depth that is within a range defined by any two of the preceding values. A depression can have a depth, for example, from 3 μm to 150 μm. The depression can have any suitable cross-sectional profile such as, for example, round, oval, slit, oblong, or can have an irregular contour. The edges of the depression can be smoothed or chamfered to facilitate fluid coupling to the fenestration and/or to improve comfort.

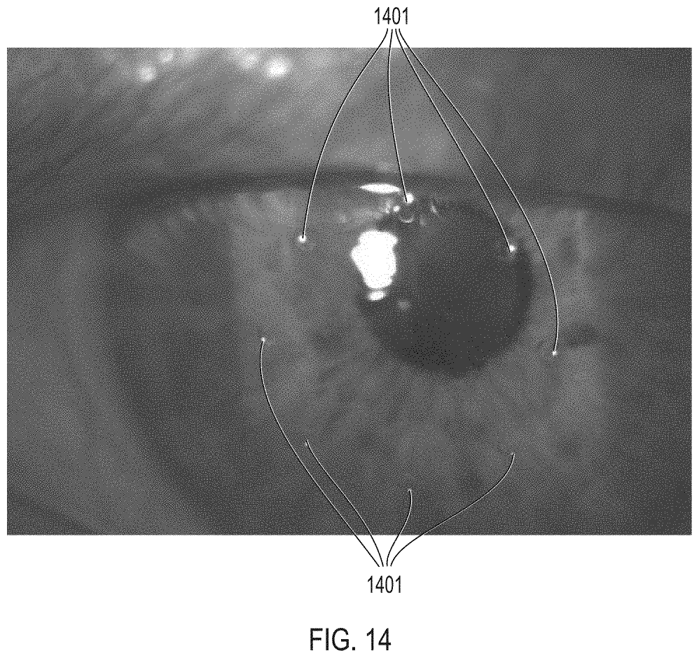

For example, FIGS. 15A-15H show views of a dynamic contact lens having depressions and fenestrations within the depressions disposed in the second peripheral portion near the transition zone. FIGS. 15A and 15B show views of the anterior surface and a cross-sectional view, respectively, of a dynamic contact lens. The dynamic contact lens shown in FIGS. 15A and 15B includes first peripheral portion 1501, second peripheral portion 1502, optical portion 1503, transition zone 1506, fenestration 1504 within depression 1507, and posterior groove 1505. FIG. 15C shows a magnified cross-sectional view illustrating the depression 1507 and fenestration 1504, which are coupled to a groove 1505 in the posterior surface of the contact lens. FIG. 15C shows a depression 1507 and fenestration 1504 in peripheral portion 1502 coupled to posterior groove 1505.

FIG. 15E shows a view of the posterior surface of a dynamic contact lens including first peripheral portion 1501, second peripheral portion 1502, optical portion 1503, and depression 1507 with a fenestration 1504. FIG. 15F shows the anterior surface of the dynamic contact lens shown in FIG. 15E including first peripheral portion 1501, second peripheral portion 1502, optical portion 1503, and depression 1507 with a fenestration 1504. FIG. 15G shows a view of the posterior surface of a dynamic contact lens including first peripheral portion 1501, second peripheral portion 1502, optical portion 1503, and groove 1505 with a fenestration 1504. FIG. 15H shows the anterior surface of the dynamic contact lens shown in FIG. 15G including first peripheral portion 1501, second peripheral portion 1502, optical portion 1503, and depression 1507 with a fenestration 1504.

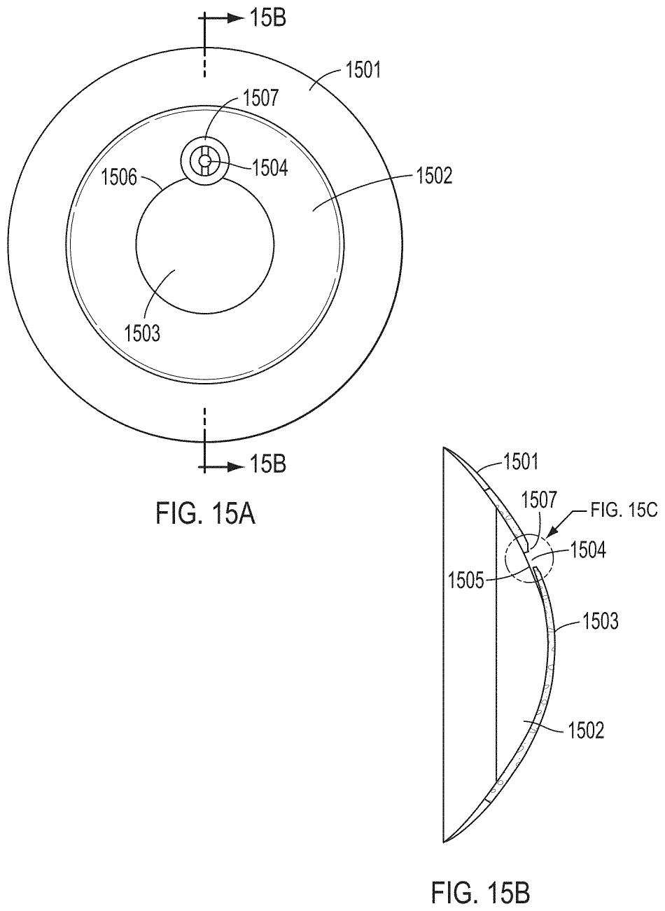

Alternatively, or in addition to a depression, a fenestration can be fluidly coupled to grooves on the anterior surface of the peripheral portion configured to draw fluid from the tear meniscus toward and into the fenestration by capillary forces. Examples of these structures are shown in FIGS. 16A-16C. FIGS. 16A-16C show side, perspective, and cross-sectional views, respectively, of a dynamic contact lens having a first peripheral portion 1601, a second peripheral portion 1602, an optical portion 1603, and a cavity 1604 in the anterior surface of the second peripheral portion 1602 with a fenestration 1605 in the bottom of the cavity 1604. As shown in FIG. 16B, on the posterior surface, a groove 1606 is coupled to the fenestration 1605 and extends from the second peripheral portion 1602 into the optical portion 1603. A cross-sectional view of the dynamic contact lens is shown in FIG. 16C, and in addition the elements shown in FIGS. 16A-16B, shows that the posterior groove 1606 narrows toward the optical portion 1603 and is fluidly coupled to optical tear volume 1607.

A mechanism for facilitating tear fluid transport and control of tear fluid transport can include one or more protrusions. The one or more protrusions can be disposed on the anterior surface of the peripheral portion of the dynamic contact lens. The one or more protrusions can be configured to facilitate interaction of an eyelid with the dynamic contact lens and can serve to amplify the force applied to the dynamic contact lens by an eyelid.

A protrusion can be configured to amplify a mechanical force applied by an eyelid such as a pushing force toward the optical portion. The pushing force can serve to destabilize or stabilize a quasi-stable configuration of the optical portion.

A protrusion can be associated with another mechanism for facilitating or controlling the transport of tear fluid. For example, a protrusion can be located in proximity to and mechanically coupled to a groove, a fenestration, and/or a tear fluid reservoir such that a force applied to the protrusion by an eyelid is transferred to an anterior groove, a posterior groove, a fenestration, a cavity, and/or tear fluid reservoir. For example, a protrusion can be located at a peripheral edge of a mechanism such as a tear fluid reservoir such that eyelid motion against the protrusion causes tear fluid to be pushed toward and into the optical tear volume.

The location and dimensions of a protrusion can be selected to minimize or avoid patient discomfort while also serving the intended function of controlling tear fluid flow.

A protrusion may have a height of at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. A protrusion may have a height of at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. A protrusion may have a height that is within a range defined by any two of the preceding values. A protrusion can have a height, for example, from 10 μm to the 600 μm, from 20 μm to 500 μm, from 50 μm to 400 μm, or from 100 μm to 300 μm.

A protrusion may have a width of at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, 2,000 μm, 3,000 μm, 4,000 μm, 5,000 μm, or more. A protrusion may have a width of at most about 5,000 μm, 4,000 μm, 3,000 μm, 2,000 μm, 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. A protrusion may have a width that is within a range defined by any two of the preceding values. A protrusion can have a width, for example, from 20 μm to 3,000 μm, from 50 μm to 2,500 μm, from 100 μm to 2,000 μm, from 200 μm, to 1,500 μm, or from 400 μm to 1,000 μm. or even larger and encompass substantial part of the lens.

A protrusion can have any suitable shape. For example, a protrusion can be round, oval, oblong, elongated, or annular.

A protrusion can comprise a plurality of protrusions. The plurality of protrusions can be located symmetrically or non-symmetrically around the optical portion of the lens. The plurality of protrusions can be located at a single radial distance from the center axis of the lens or can be located at different radial distances from the center axis of the lens.

A mechanism for facilitating tear fluid transport and controlling tear fluid transport can include one or more valves. A valve can be associated with another tear fluid control mechanism such as a groove, fenestration, and/or tear fluid reservoir.

A valve can be configured to control the direction of tear fluid flow. For example, a valve can allow fluid to flow from a tear fluid reservoir into an optical tear volume, and resist or prevent the flow of tear fluid from the optical tear volume into the tear fluid reservoir.

A valve can be configured to provide a variable resistance to tear fluid flow. For example, a valve can resist tear fluid transport at a first tear fluid pressure and allow tear fluid transport at a second pressure.

A valve can be sensitive to mechanical forces such that a mechanical force applied by an eyelid can cause the valve to open or close. The mechanical force can be amplified by one or more protrusions.

A valve can be bidirectional or can be unidirectional.

A valve can include a capillary valve that is configured to control tear fluid flow based on a pressure difference between the tear fluid in the optical tear volume and the tear fluid pressure within an tear fluid reservoir, the tear fluid pressure in the vicinity of a fenestration, and/or the tear fluid pressure in an anterior groove and/or a posterior groove.

A valve can include a capillary valve that is configured to control tear fluid flow based on the shape, size, and length of fenestration and/or groove

A valve can be configured such that the valve seals the area on the posterior surface of the dynamic contact lens to prevent the flow of tear fluid and opens to allow the flow of tear fluid.

Examples of other suitable valves include “fish-mouth” valves or a slit membrane. Examples of other suitable valves include a capillary valve in which the capillary forces between the tear fluid and the walls of a fenestration near the anterior orifice and the air allow tear fluid to flow toward the optical tear volume. To achieve the required valve properties suitable lens materials, coatings or treatments, fenestration shape, and fenestration dimensions can be selected. For example, the capillary forces will be greater for more hydrophilic surfaces and therefore the pressure difference that can cause a valve to open can be less. Similarly, the thinner a fenestration the higher the pressure needed to open a valve. A fenestration can have a diameter, for example, from 10 μm and 1 mm.

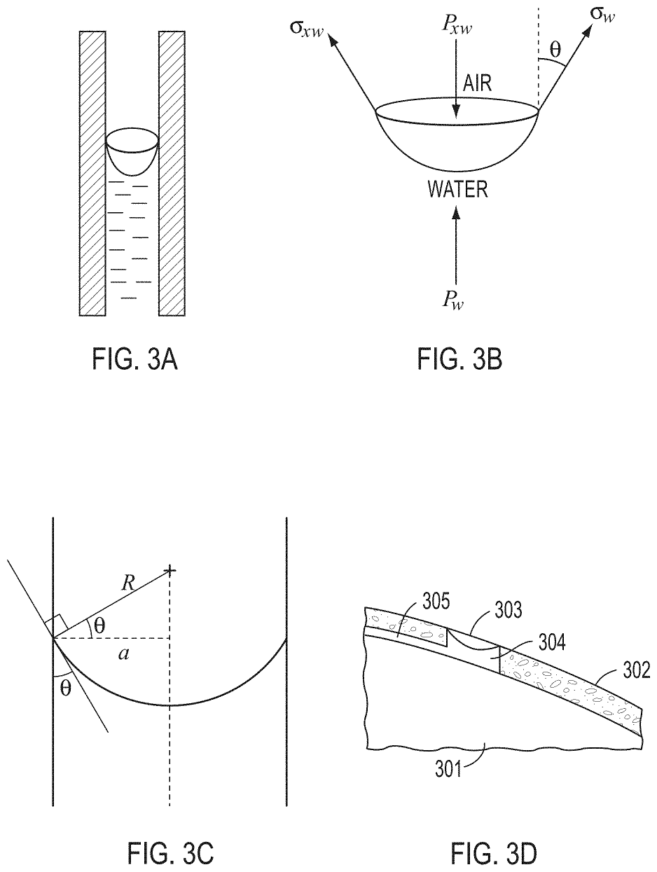

FIGS. 2A-2B show examples of valves. FIG. 2A shows a top view and FIG. 2B shows a cross-sectional view of a dynamic contact lens having a peripheral portion 201/202, a fish-mouth valve 210 disposed between the anterior and the posterior surface of the lens, which is coupled to a posterior groove 205, which is coupled to optical portion 203, to a tear fluid reservoir, or to another feature in the posterior surface of the dynamic contact lens. FIG. 2A shows a top view of the dynamic contact lens with an amplified view 204 of a sectional fish-eye valve 210 coupling the anterior surface 207 of the lens to posterior groove 205. FIG. 2B includes a detailed cross-sectional view 208 of a dynamic contact lens of an open fish-mouth valve 210.

The various mechanisms disclosed herein can be fluidly coupled by the tear film overlying the epithelial layer of the cornea. For example, it is not necessary that a tear fluid reservoir, cavity, or a fenestration be fluidly coupled to the optical tear volume by a groove. Rather, a tear fluid reservoir and the optical tear volume can be fluidly coupled by the tear film overlying the epithelial layer. Certain mechanisms can be fluidly coupled to each other by features incorporated into the posterior surface of the peripheral portion, while other mechanisms can be fluidly coupled by the tear film overlying the epithelial layer.

At the interface between the optical portion and the peripheral portion, the peripheral portion can have a peripheral base curvature from 7.5 mm to 9.5, such as from 8 mm to 9 mm, and the optical portion can have an optical base curvature that is less than the peripheral base curvature. At the interface, the optical base curvature can be more than 0.4 mm less than the peripheral base curvature. For example, the optical base curvature can be less than 0.4 mm than the peripheral base curvature, less than 0.5 mm, less than 1.0 mm, less than 1.5 mm, or less than 2.0 mm than the peripheral base curvature. For example, the optical base curvature can be from 0.4 mm to 2 mm less than the peripheral base curvature, from 0.5 mm to 1.5 mm less, from 0.75 mm to 1.0 mm less than the peripheral base curvature. The optical base curvature can be, for example, less than 7.4 mm, less than 7.3 mm, less than 7.2 mm, less than 7.1 mm, less than 7.0 mm, less than 6.9 mm, less than 6.8 mm, less than 6.7 mmm, less than 6.6 mm, less than 6.5 mm, less than 6.0 mm, less than 5.0 mm or less than 4.0 mm. The optical base curvature can be, for example, from 4 mm to 6.8 mm, from 5 mm to 6.5 mm, or from 5.5 mm to 6.0 mm. The optical base curvature can be, for example, from 4 mm to 7.4 mm, from 5 mm to 7.1 mm, or from 6.9 mm to 7.4 mm.

The interface between the peripheral portion and the optical portion can define a transition zone. The transition zone can be located at a radial distance of from 1 mm to 8 mm, such as from 1.5 mm to 7 mm, or from 1.5 mm to 5 mm, from 1.5 mm to 4 mm, or from 1.5 mm to 2.5 mm, from the center of the dynamic contact lens. The width of the transition zone can be, for example, from 0.1 mm to 2 mm, from 0.2 mm to 1.5 mm, from 0.3 mm to 1 mm, or from 0.4 mm to 0.8 mm.

The transition zone can have a transition base curvature that is different than the transition base curvature of the peripheral portion and that is different than the base curvature of the optical portion. The transition zone can have one or more transition base curvatures.

The interface can have a substantially constant thickness around the circumference.

The interface can have a variable thickness around the circumference. The thickness can vary in a regular pattern or in an irregular pattern about the circumference of the interface.

For example, the interface can include a plurality of grooves disposed in the posterior surface of the contact lens around the interface circumference. For example, the plurality of grooves can include from 3 to 16 grooves disposed symmetrically or asymmetrically across the interface. At least some of the plurality of grooves can be coupled to a fenestration, a tear fluid reservoir, or to both a fenestration and a tear fluid reservoir.

At the interface between the peripheral portion and the optical portion, the interface can be chamfered. For example, rather than have an abrupt interface, the interface can be smoothed, rounded, and/or lifted above the surface of the cornea. In other words, the interface between the peripheral portion and the optical portion can be gradual. The interface transition can be configured to enhance patient comfort.

A pre-fabricated SAG built into the optical portion can serve as mechanism for pumping tear fluid into and out of the optical tear volume.

In a dynamic contact lens, the central optical portion is designed to have a pre-fabricated sagittal height with a base curvature with is from a few microns to a few hundred microns less than that of the peripheral posterior base curvature (8.2 mm to 9.2 mm). For example, the optical portion can have an optical posterior base curvature that is from 0.1 mm to 2.5 mm less than that of the posterior base curvature of the peripheral portion, which is approximately the curvature of the anterior surface of the cornea.

When the dynamic contact lens is placed on the cornea, the pre-fabricated sagittal height provides a structural strength (rigidity) such that if tear fluid is available in proximity to the optical portion, then the tear fluid will tend to flow under the optical portion to create a lenticular volume of tear fluid between the posterior surface of the optical portion and the anterior surface of the cornea.

The ability of a pre-fabricated sagittal height to provide a pumping force is in part determined by the structural strength of r optical portion. Parameters that influence the strength include the diameter of the optical portion, which can be from 1 mm to 9 mm, the rigidity of the lens, which is determined by the thickness, which can range from 40 μm to 800 μm, the material modulus, which can range from 0.1 MPa to 8 MPa, and the radius of curvature of the optical portion.

A dynamic contact lens with an optical portion having mechanical properties that allow it to assume a continuum of geometric configurations depending on the pressure applied to the optical portion. The pressure may be applied to the anterior surface or to the posterior surface of the optical portion. In a lowest-pressure configuration, the optical portion assumes a neutral geometric configuration such that a lenticular tear volume forms between the posterior surface of the optical portion and the anterior surface of the cornea. When exposed to a negative pressure, or to a positive pressure on the anterior surface, the posterior surface of the optical portion substantially conforms to the anterior surface of the cornea such that the thickness of the tear film is substantially constant between the posterior surface of the optical portion and the anterior surface of the cornea. For example, in substantially conforming configurations, the thickness of the tear film can vary by less than 10 μm or less than 3 μm. A dynamic contact lens can also assume any suitable configuration between a fully conforming configuration and the neutral configuration depending on the magnitude of pressure applied to the optical portion of the dynamic contact lens. For a given pressure, the extent to which the optical portion conforms to the anterior surface of the cornea and the tear volume can depend on several parameters including, for example, the diameter, the thickness, the rigidity, and the sagittal depth of the optical portion; the geometry of the transition zone between the optical portion and the peripheral portion; and the elastic modulus of the lens material. For example, a dynamic contact lens provided by the present disclosure can assume a full range of configurations when exposed to a negative posterior pressure, for example, from 5 Pa to 1,500 Pa, such as from 10 Pa to 1,000 Pa, from 10 Pa to 500 Pa, from 10 Pa to 300 Pa, from 10 Pa to 200 Pa, from 10 Pa to 100 Pa, from 10 Pa to 50 Pa, from 50 Pa to 150 Pa, from 50 Pa to 250 Pa, from 50 Pa to 500 Pa, from 100 Pa to 250 Pa, from 100 Pa to 500 Pa, from 100 Pa to 750 Pa, or from 100 Pa to 1,000 Pa. When the negative pressure is relieved, the mechanical properties of the lens are such that the lens returns to the neutral configuration with a maximum lenticular volume between the posterior surface of the optical portion and the anterior surface of the cornea.

Two main forces are acting against the pumping pressure generated by the sagittal height and the other parameters of the optical portion.

First, there is a counteracting suction force. When there is only a very thin layer of tear fluid between the central optical portion of a dynamic contact lens and the cornea, such as a tear film less than 5 μm thick, there is an adhesion force between the contact lens and the cornea. The smaller the diameter and/or the thinner the lens the higher the suction force.

Second, there are counteracting capillary forces. When tear fluid is available and can flow into the tear volume through grooves or other features, tear fluid will flow into the optical tear volume. When the grooves are then, tear fluid transport is believed to be dominated by capillary forces. A capillary force can be generated by grooves connected external to the lens through fenestrations. The number, geometry, and dimensions of the grooves can determine the strength of the capillary forces. For example, for shorter grooves and larger the dimensions of the fenestrations, the lower the capillary forces tend to be.

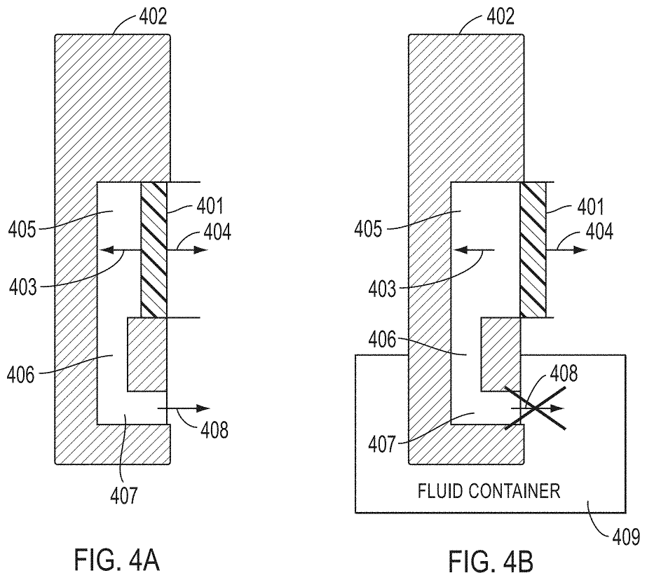

The parameters associated with capillary forces within fenestration are illustrated in FIGS. 3B-3C. FIG. 3A shows the meniscus that is being created inside a fenestration. FIGS. 3B and 3C show a cross-sectional view of tear fluid within a fenestration and the parameters associated with the meniscus. The pressure across the meniscus is related to the radius and the surface tension γ by the equation Δp=2γ/R. The definitions of the parameters are illustrated in FIG. 3B and in FIG. 3C.

The counteracting capillary forces can be controlled by fluidly coupling the optical tear volume to a source of tear fluid such as the tear meniscus. Having a fenestration with a conduit that enters or is in proximity to the optical portion not only serves to couple the optical portion to a source of tear fluid, but can also be configured to function as a capillary valve such that when the fenestration is fluidly coupled to the tear meniscus the capillary forces are reduced and tear fluid can flow into the tear volume driven in part by the pumping force generated by the pre-fabricated sagittal height. Then, when the fenestration is open to the air or is buried below the eyelid and not coupled to the tear meniscus, the fenestration functions as a closed valve that prevents tear fluid from flowing into the optical portion.

Adhesion forces can also be reduced by surface treatment. For example, a surface of the contact lens can be treated with a hydrophobic coating to reduce adhesive force. The hydrophobic coating or treatment can be applied to a portion of the posterior surface of the lens. A hydrophilic coating or treatment can also be applied to a portion of the posterior surface of the lens to increase the adhesion force between the lens and the cornea and to reduce the mobility of the contact lens on the eye.

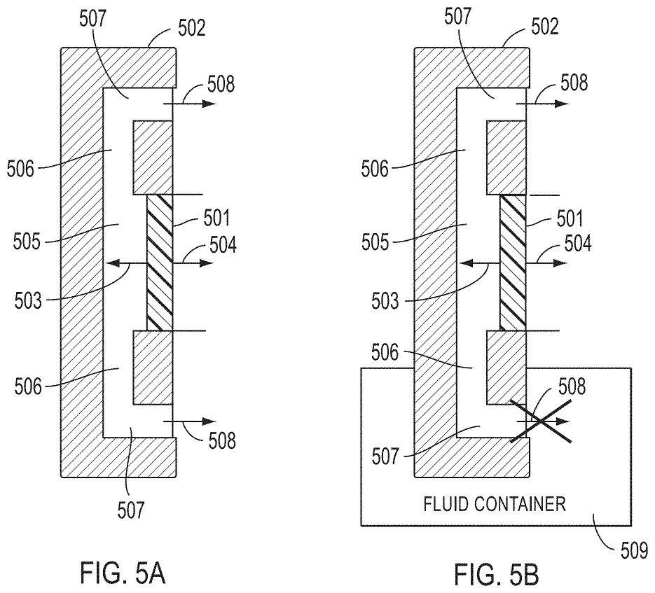

FIGS. 4A-4B show a fluid dynamic models of tear fluid transport in a dynamic contact lens having a single fenestration, which is either or open to air or is fluidly coupled to a tear meniscus. In FIG. 4A the piston 401 represents the optical portion showing a suction force 403 pulling the optical portion 401 toward the cornea 402 and a restoring force 404 tending to pull the optical portion 401 away from the cornea 402. The restoring force 404 is generated by the structure of the optical portion such as the pre-fabricated sagittal height. An optical tear volume 405 is situated between the optical portion 404 and the cornea 402 and as shown in FIG. 4A is fluidly coupled by a groove 406 and to a fenestration 407. Capillary forces 408 generated within the fenestration 407 pull the tear fluid away from the optical tear volume 405 and may act similar to a closed valve. In FIG. 4B the fenestration 407 is fluidly coupled to a source of tear fluid 409 such as a tear meniscus. Fluid coupling of the fenestration 407 to the source of tear fluid cancels the capillary force 408 and may act similar to an open valve such that the sum of the forces causes the optical portion 401 represented by the piston to overcome the suction force 403 and to pull away from the cornea 402 and thereby cause an increase in the optical tear volume 405.

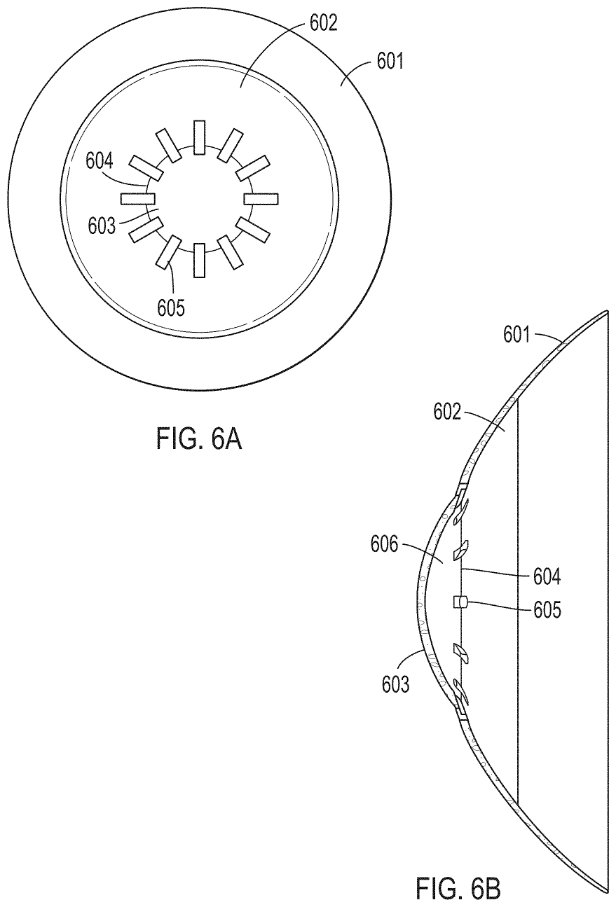

FIGS. 5A-5B show another fluid dynamic model of tear fluid transport in a dynamic contact lens having two fenestrations 507. As shown in FIG. 5A, the position of the optical portion 501 represented by the piston is determined by a suction force 503, a structural force 504, and by the capillary forces 508 within the two fenestrations 507. When one or both of the fenestrations 507 are fluidly coupled to a source of tear fluid 509 as shown in FIG. 5B, the position of the optical portion 501 moves away from the cornea 502 causing the optical tear volume 505 to increase. Fenestrations 507 are fluidly coupled to optical tear volume 505 by grooves 506.

The behaviour of valves is mainly dictated by the valve opening pressure which is the maximum pressure that the valve can hold before it opens. The valve opening pressure is a function of the geometry and the materials involved such as the valve material and the fluid. For example, the larger the valve opening the smaller the valve opening pressure is. The length of the valve opening and how valve geometry changes during opening can also influence the valve behaviour. For example, the valve can have a stepped geometry that creates different valve opening pressures. This geometry can be used to allow incremental fluid flow while increasing the opening pressure through the valve. The cross-sectional geometry of a valve can also influence the opening pressure. The interaction between the lens material and the tear fluid can be associated with the influence on surface tension, contact angle, and the adhesion energy. For example, the greater the surface tension or the smaller the contact angle the higher will be the capillary force and as a result the capillary pressure over the valve. To calculate the pressure of fluid in a capillary conditions Jurin's Law that defines the height h of a liquid column:

h=(2γ cos θ)/(ρgr)

where γ is the liquid-air surface tension (force/unit length), θ is the contact angle, ρ is the density of fluid (mass/volume), g is the local acceleration due to gravity (length/square of time^{[28]}), and r is the radius of tube. Therefore, the thinner the space in which the fluid can travel, the greater the capillary force. The relationship can be changed by using coatings to alter wettability or hydrophilic properties of the surfaces.

The interface between the optical portion and the peripheral portion can be configured to facilitate the ability of the contact lens to transition between quasi-stable configurations and/or to maintain quasi-stable configurations. This interface can be referred to as the transition zone.

At least a portion of the transition zone can have a thickness that is less than the thickness of the peripheral portion and the thickness of the optical portion at the respective interfaces with the transition zone.

The transition zone may have a circumference that is not uniform throughout the entire circumference. For example, certain regions of the transition zone can be thinner and/or have a different base curvature than other regions of the transition zone. Certain regions of the transition zone can have a different thickness and/or a different base curvature than that of the peripheral portion and/or optical portion. For example, thickness of certain regions of the transition zone can be thinner than the adjacent region of the peripheral portion and/or the optical portion by from 10 μm to 300 μm, from 20 μm to 200 μm, or from 50 μm to 150 μm, depending on the thickness of the adjacent region of the peripheral portion and/or the optical portion. For example, the transition zone can have a base curvature that differs from the base curvature of the peripheral portion and/or the optical portion by from 100 μm to 5 mm, from 200 μm to 4 mm, from 300 μm to 3 mm, or from 500 μm to 2 mm.

The transition zone can have a base curvature that is different than the base curvature of the peripheral portion and that is different than the base curvature of the optical portion; and ay least a portion of the transition zone can have a thickness that is less than the thickness of the peripheral portion and the thickness of the optical portion at the respective interfaces with the transition zone.

The transition zone can have a transition base curvature that is less than the peripheral base curvature and greater than the optical base curvature. The transition zone can have a transition base curvature that is less than the peripheral base curvature and that is less than the optical base curvature.

The transition zone can have a base curvature that is different than both the peripheral base curvature and the optical base curvature.

The transition zone can have a thickness that is substantially the same throughout the circumference of the transition zone.

The transition zone can have a thickness that varies throughout the circumference of the transition zone.

The transition zone can have a thickness that varies in a regular pattern throughout the circumference of the transition zone.

The transition zone can have a thickness that varies in an irregular pattern throughout the circumference of the transition zone.

The transition zone may have a width of at least about 10 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1,000 μm, or more. The transition zone may have a width of at most about 1,000 μm, 950 μm, 900 μm, 850 μm, 800 μm, 750 μm, 700 μm, 650 μm, 600 μm, 550 μm, 500 μm, 450 μm, 400 μm, 350 μm, 300 μm, 250 μm, 200 μm, 150 μm, 100 μm, 90 μm, 80 μm, 70 μm, 60 μm, 50 μm, 40 μm, 30 μm, 20 μm, 10 μm, or less. The transition zone may have a width that is within a range defined by any two of the preceding values. The transition zone can have a width, for example, from 10 μm to 2 mm, from 50 μm to 1.5 mm, from 100 μm to 1 mm, or from 250 μm to 750 μm.

The transition zone can have, for example, a plurality of grooves disposed in the posterior surface of the contact lens. For example, the transition zone can comprise from 3 to 16 grooves, such as from 6 to 12 grooves disposed symmetrically or asymmetrically about the circumference of the transition zone.

At least some of the grooves can be configured to transport tear fluid into and out of the tear volume. At least some of the plurality of grooves can be coupled to a fenestration, to a tear fluid reservoir, or to both a fenestration and a tear fluid reservoir.

The radius of curvature of the transition zone and the thickness of the transition zone can be configured to facilitate the transition between quasi-stable configurations of the dynamic contact lens and/or to maintain quasi-stable configurations of the contact lens.

The transition zone can have a radius of curvature that is the same as either the peripheral base curvature, or the same as the optical base curvature, and can have a thickness that is greater than the thickness of the thickness of the peripheral portion and the optical portion at the interface with the transition zone, or can have a thickness that is less than the thickness of the thickness of the peripheral portion and the optical portion at the interface with the transition zone.

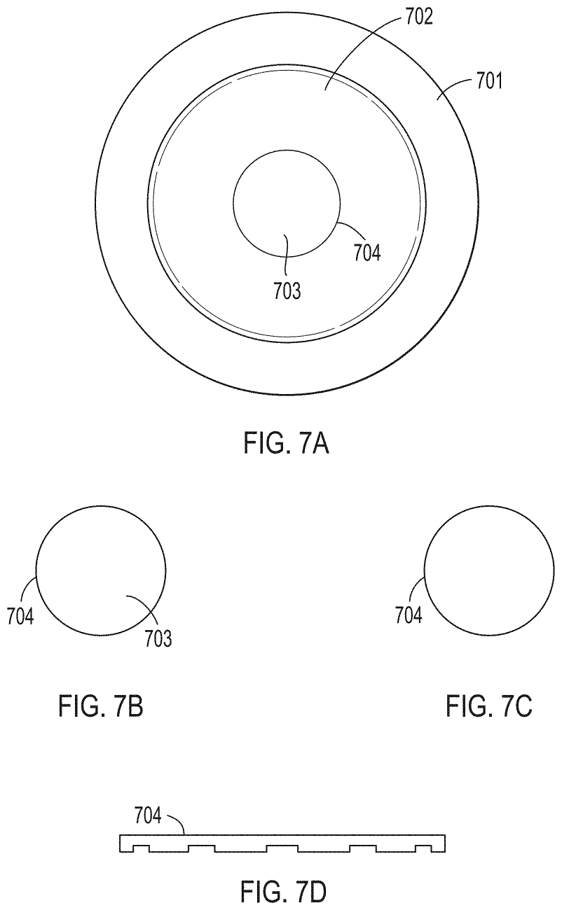

FIGS. 6A and 6B show a view of an anterior surface and a cross-sectional view, respectively, of an example of a dynamic contact lens provided by the present disclosure having an abrupt transition zone with discontinuities. The dynamic contact lens includes first peripheral portion 601, second peripheral portion 602, optical portion 603, and abrupt transition zone 604. As shown in the cross-sectional view of FIG. 6B, the abrupt transition zone is characterized by discreet difference in the base curve of the second peripheral portion 602 and the base curve of the optical portion 603 and the interface 604 between the two regions. Channels or grooves 605 are shown to extend from the peripheral portion across the abrupt transition zone 604 into the optical portion 603 and represent discontinuities around the circumference of the abrupt transition zone 604.

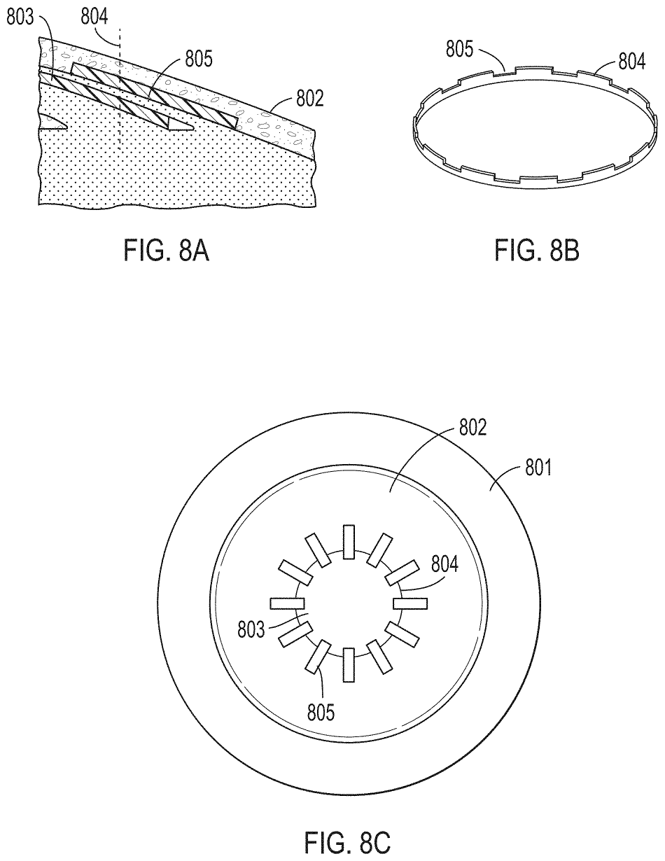

Additional examples of transition zone discontinuities are shown in FIGS. 7A-7D.

FIGS. 7A-7D show an example of a dynamic contact lens having a first peripheral portion 701, a second peripheral portion 702, an optical portion 703 and transition zone 704 at the interface between the second peripheral portion 702 and the optical portion 703. As shown in FIG. 7D, the transition zone 704 can have a discontinuous cross-sectional profile such that the thickness varies in a regular manner around the circumference of the transition one. The differing thickness can be associated with grooves in the posterior surface of the dynamic contact lens that transection the transition zone. In other embodiments, the discontinuities can be irregular. FIG. 7B shows a view of the optical portion 703 and the circumference of the transition zone 704. FIG. 7C shows a top view of the abrupt transition zone 704.

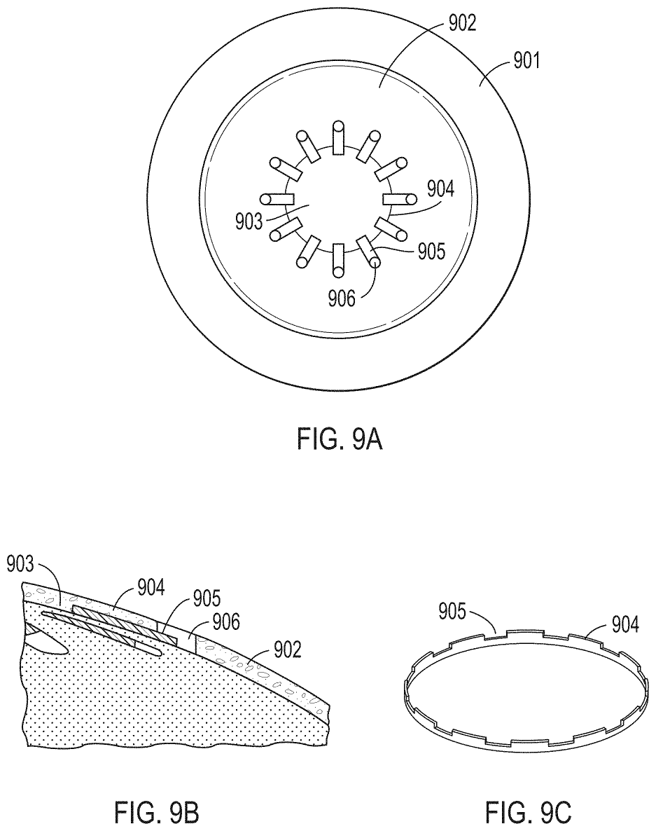

FIGS. 8A-8C show similar views of a dynamic contact lens having an abrupt transition zone, but with discontinuities in the posterior surface of the dynamic contact lens and extending across the abrupt transition zone. The dynamic contact lens shown in FIGS. 8A-8C include first peripheral portion 801, second peripheral portion 802, optical portion 803, and abrupt transition zone 804. The abrupt transition one 804 includes irregularities 805 such as posterior grooves extending across the transition zone 804 such that the transition zone has a different thickness around the circumference.

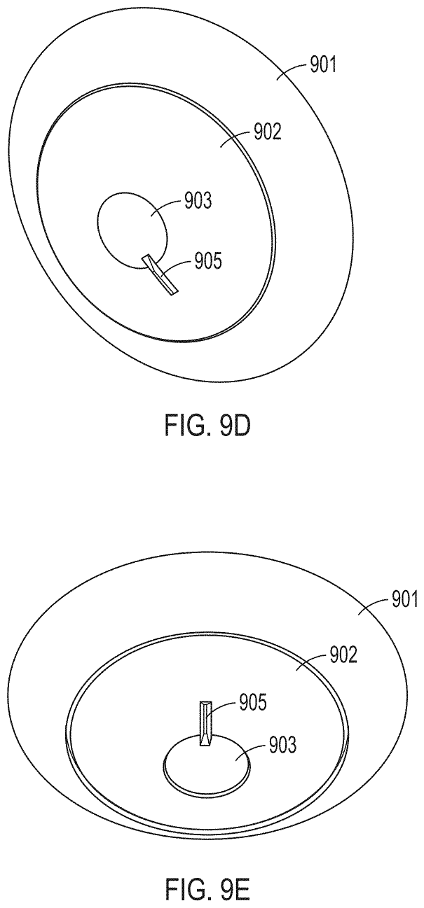

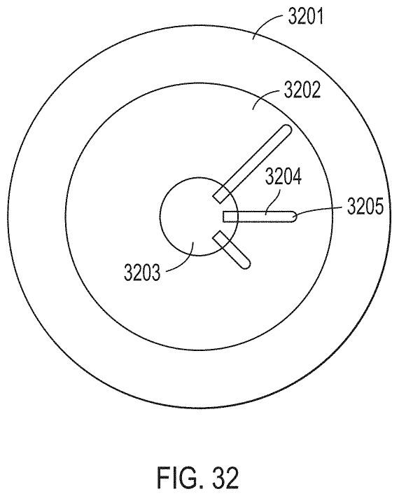

The dynamic contact lens shown in FIGS. 9A-91 include first peripheral portion 901, second peripheral portion 902, optical portion 903, and abrupt transition zone 904. The abrupt transition zone 904 includes irregularities 905 such as grooves extending across the transition zone such that the transition zone 904 has a different thickness around the circumference. One end of each groove 905 is connected to a fenestration 906 and extends into optical region 903.

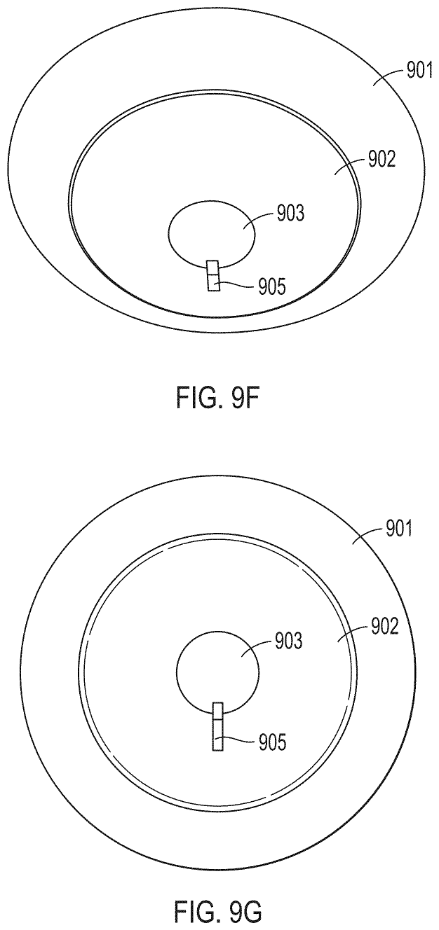

As an example, FIG. 10 shows a posterior surface of a dynamic contact lens provided by the present disclosure including an optical portion 1006, a first peripheral portion 1003, a second peripheral portion 1001, and a transition zone 1002. The dynamic contact lens includes radial grooves 1004 extending from the second peripheral portion 1001 to the transition zone 1002, and a fenestration 1005 coupled to each of the grooves 1004. As shown in FIG. 10, groove 1004 terminates at the transition zone 1002.

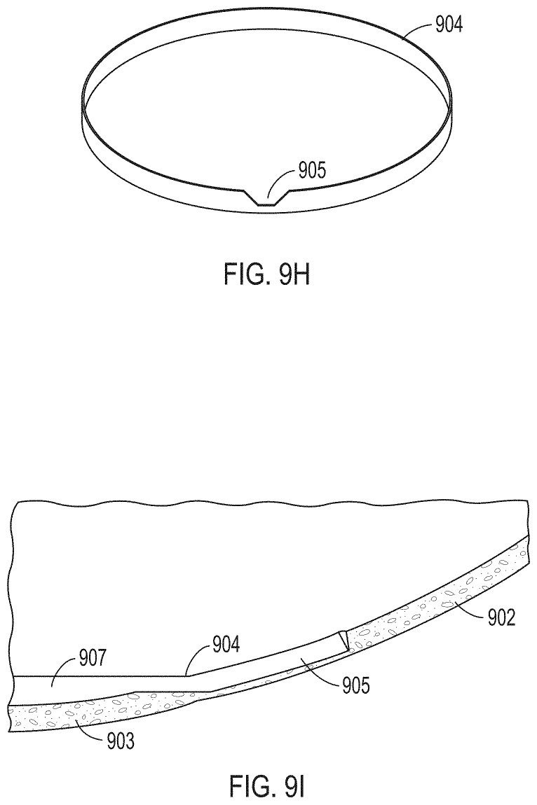

FIG. 11 shows an anterior surface of another dynamic contact lens provided by the present disclosure including optical portion 1101, transition zone 1102, and peripheral portion 1103. The dynamic contact lens also includes 8 fenestrations through the peripheral portion of the dynamic contact lens. As shown in FIG. 11, groove 1104 terminates at the transition zone 1102.

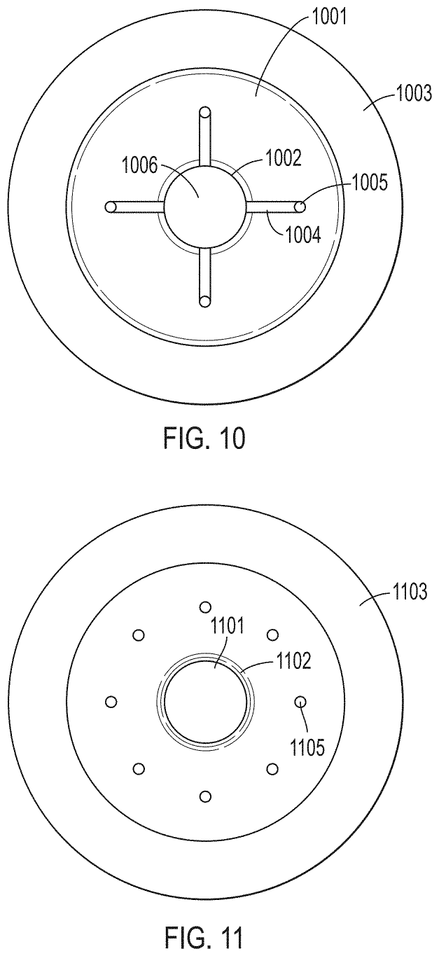

FIG. 12 shows the posterior surface of the same contact lens as shown in FIG. 11 including optical portion 1201, peripheral portion 1203, radial posterior grooves 1204 and fenestrations 405 connected to each of the posterior grooves 1204.

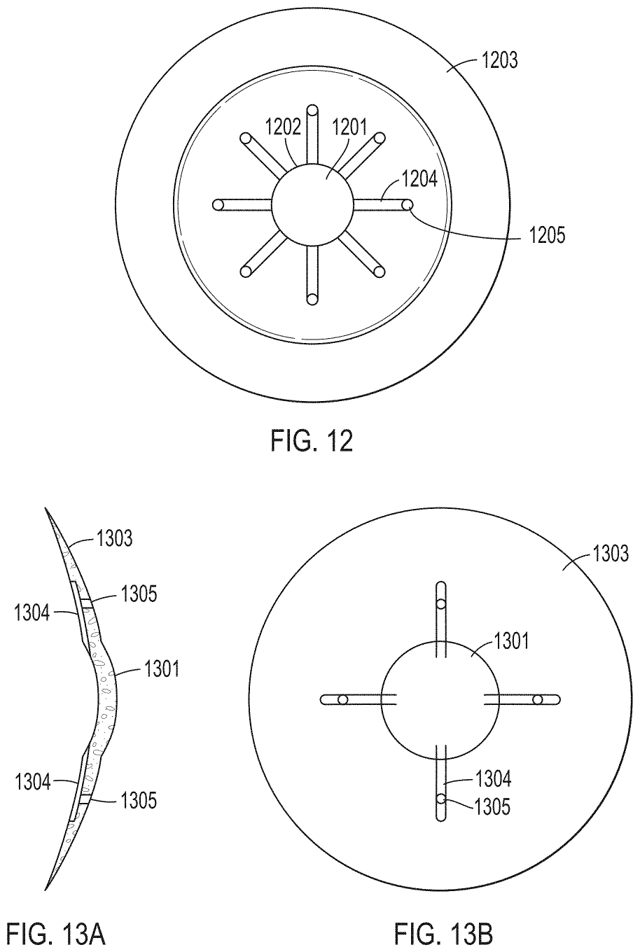

FIG. 13A shows a cross-sectional view of an example of a dynamic contact lens provided by the present disclosure including optical portion 1301 peripheral portion 1303, radial posterior grooves 1304 and fenestrations 1305. A view of the posterior surface of the same dynamic contact lens is shown in FIG. 13B and including optical portion 1301, peripheral portion 1303, radial posterior grooves 1304 and fenestrations 1305. As shown in FIGS. 13A and 13B, the radial posterior grooves extend into the posterior surface of the optical portion 1301 or, as shown in FIG. 12, may terminate at the interface of the peripheral portion with the optical portion.

FIG. 13C shows the dynamic contact lens of FIGS. 13A and 13B on the eye of a patient and includes optical portion 1301, peripheral portion 1303, transition zone 1302, four radial posterior grooves 1304, and a fenestration 1305 connected to each of the posterior grooves 1304.

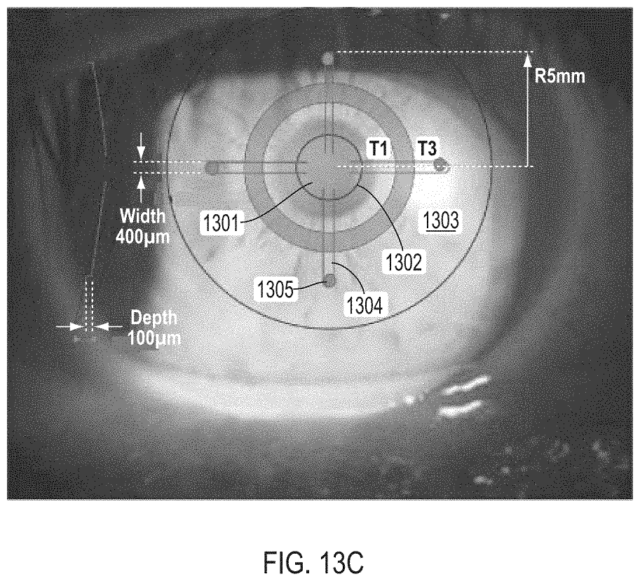

FIG. 14 shows a slit lamp bio-microscope image of a dynamic contact lens having eight (8) fenestrations on an eye of a patient. The fenestrations 1401 are visible as eight (8) white dots.

From a functional perspective, the dynamic contact lens can be configured such that with forward gaze, the optical portion is closest to the cornea, and in downward gaze the optical portion bulges away from the cornea. During forward gaze the fenestrations are not fluidly couple to a source of tear fluid. During downward gaze, the fenestrations become fluidly coupled to the tear meniscus allowing tear fluid to flow into the optical tear volume causing the optical portion to bulge outward and away from the cornea to increase the optical power of the anterior optical surface.

In the simplest form, a dynamic contact lens can have an optical portion with a pre-fabricated central sagittal height and fenestrations that are located on peripheral portion. During primary gaze, the fenestrations do not come in contact with the tear meniscus and therefore no fluid is available to fill the optical tear volume. During downgaze, one or more fenestrations can come in contact with the tear meniscus, which enables tear fluid to flow into the optical tear volume between the posterior surface of the optical portion and the anterior surface of the cornea.

A mechanism for inducing a change in conformation can comprise manipulating tear fluid reservoirs and/or tear fluid cavities.

Reservoirs can be formed in the posterior surface of a dynamic contact lens. The reservoirs can be disposed in the peripheral portion of the lens and outside the optical region so as not to interfere with vision. The reservoirs can be compressible or non-compressible.

When applied to an eye, the reservoirs can fill with tear fluid to form tear fluid reservoirs. The tear fluid reservoirs can be compressible or non-compressible. A dynamic contact lens can comprise compressible tear fluid reservoirs, non-compressible tear fluid reservoirs, or a combination thereof.

A tear fluid reservoir can be compressible by application of eyelid pressure. The eyelid pressure can be applied, for example, by changing the gaze angle of the eye, by normal blinking, by intentionally blinking, by squinting, or by a combination of any of the foregoing.

A tear fluid reservoir may be compressed by a force of at least about 0.1 μm-force, 0.2 μm-force, 0.3 μm-force, 0.4 μm-force, 0.5 μm-force, 0.6 μm-force, 0.7 μm-force, 0.8 μm-force, 0.9 μm-force, 1 μm-force, 2 μm-force, 3 μm-force, 4 μm-force, 5 μm-force, 6 μm-force, 7 μm-force, 8 μm-force, 9 μm-force, 10 μm-force, or more. A tear fluid reservoir may be compressed by a force of at most about 10 μm-force, 9 μm-force, 8 μm-force, 7 μm-force, 6 μm-force, 5 μm-force, 4 μm-force, 3 μm-force, 2 μm-force, 1 μm-force, 0.9 μm-force, 0.8 μm-force, 0.7 μm-force, 0.6 μm-force, 0.5 μm-force, 0.4 μm-force, 0.3 μm-force, 0.2 μm-force, 0.1 μm-force, or less. A tear fluid reservoir may be compressed by a force that is within a range defined by any two of the preceding values. A tear fluid reservoir can be compressed by a force within a range, for example from 0.1 μm-force to 10 μm-force, from 0.2 μm-force to 8 μm-force, from 0.5 μm-force to 6 μm-force, from 1 μm-force to 5 μm-force, or from 2 μm-force to 4 μm-force.

To be effective in inducing a change in conformation of the optical portion, it may only be necessary that the tear fluid reservoir can be partially compressible. For example, to induce a change in conformation, an amount of tear fluid can be forced into the tear film gap between the posterior surface of the optical portion and the cornea. The amount of tear fluid can be sufficient to widen the gap or otherwise weaken the capillary force and release the capillary adhesion. Subsequently, as the optical portion transitions to a non-conforming configuration, tear fluid fills the expanding lenticular volume and at least some of the tear fluid can be drawn from the tear fluid reservoirs. Alternatively, or in addition, tear fluid can be intermittently, continuously, or semi-continuously forced into the gap between the optical posterior surface and the cornea by applying eyelid pressure to the tear fluid reservoir and/or by movement of the eye to provide one or more discrete non-conforming configurations or one or more continuous non-conforming configurations.

Tear fluid reservoirs can also be involved in a mechanism for transitioning from a non-conforming configuration to a conforming configuration. When released from a fully compressed or partially compressed state, a tear fluid reservoir can be configured to expand. The expanding lenticular volume of the tear fluid reservoir can draw tear fluid from the tear film and from the tear volume. The result of filling the tear fluid reservoirs can be to pull the posterior surface of the optical portion against the cornea to establish or to restore a quasi-stable state of the conforming configuration.

The one or more tear fluid reservoirs can be configured to compress when pressure is applied by an eyelid, only during a gaze change. During a gaze change, pressure applied by an eyelid to the anterior surface of the cornea and/or to a compressible tear fluid reservoir can provided by the anterior surface coming into dynamic contact with an eyelid. More force can be applied to a compressible tear fluid reservoir by normal blinking, by intentional blinking, and/or by squinting where the squinting can be held for a certain period of time and with a certain force with the eyes closed.

Thus, the at least one first mechanism, the at least one second mechanism, or both the at least one first mechanism, and the at least one second mechanism can comprise manipulating fluid within one or more tear fluid reservoirs. The tear fluid reservoirs can be fluidly coupled to the tear film or to the tear volume between the posterior portion and the cornea by a tear film between the posterior surface of the peripheral portion and the cornea.

A reservoir can be configured such that during compression tear fluid is preferentially pushed beneath the optical portion and when released tear fluid is preferentially drawn from beneath the optical portion of the dynamic contact lens. This can be accomplished, for example, with appropriate selection of the shape of the cavity/tear fluid reservoir. For example, a suitable shape can comprise a cross-sectional profile that narrows toward the optical portion such as a wedge-shaped cavity/tear fluid reservoir.

A dynamic contact lens can comprise one or more tear fluid reservoirs.

A single tear fluid reservoir can comprise a concentric cavity disposed at a radial distance from the center geometric axis of the dynamic contact lens. A single tear fluid reservoir can comprise a cavity disposed in only a part of the peripheral portion. For example, a single tear fluid reservoir can comprise a cavity in the shape of an arc on one half of a peripheral portion of a dynamic contact lens. For example, the arc-shaped cavity can be disposed at a radial distance from the center geometric axis of the dynamic contact lens and configured to be worn such that the arc-shaped tear fluid reservoir is on the lower portion of the dynamic contact lens when worn by a user. A single tear fluid reservoir can be configured such that the reservoir can interact with an eyelid. More than one circular reservoir can be provided such that each reservoir can have, for example, a different internal diameter. A circular reservoir can also have compartments such that when pressure is applied on the reservoir the tear fluid preferentially moves toward the optical portion and not within the circular reservoir.

A dynamic contact lens can comprise two or more tear fluid reservoirs such as a plurality of tear fluid reservoirs. The tear fluid reservoirs can be shaped and disposed in the peripheral portion as suitable to interact with one or both eyelids and to induce transitions between conforming and non-conforming configurations. Tear fluid reservoirs can be disposed symmetrically or asymmetrically around the optical portion. The tear fluid reservoirs can be disposed outside the optical zone so as not to interfere with vision.

The at least one first mechanism, the at least one second mechanism, or both the at least one first mechanism and the at least one second mechanism can comprise exchanging tear fluid by compressing the optical portion and/or compressing the peripheral portion of the dynamic contact lens, when pressure is applied to the dynamic contact lens by an eyelid or when one of the lens features interacts with the tear meniscus, during a gaze change. Exchanging tear fluid can comprise exchanging tear fluid between and/or among the tear film between the posterior surface of the optical portion and the cornea, the tear film between the peripheral posterior surface and the cornea, the tear volume, one or more tear fluid reservoirs, tear fluid at the periphery of the lens, tear fluid on the anterior surface of the lens, tear fluid from the lower and/or upper tear meniscus, or a combination of any of the foregoing.

The at least one first feature, the at least one second feature, or both the at least one first feature and the at least one second feature can comprise protrusions on an anterior surface of the dynamic contact lens configured to interact with an eyelid or when one of the lens features interacts with the tear meniscus.

The optical portion and the one or more tear fluid reservoirs can be contiguous. In this design, eyelid motion on the peripheral portion of a tear volume can cause the optical portion to move toward the cornea such that the optical portion bulges anteriorly. The optical portion can assume a conforming configuration or a non-conforming configuration when bulging anteriorly. The optical portion can assume a at least two different non-conforming configurations when bulging anteriorly.

Similar features as described for use with tear fluid reservoirs can be used without tear fluid reservoirs. A dynamic contact lens may not have reservoirs and tear fluid reservoirs and similar action by the eyelids and/or gaze angle of the eye and/or when one of the lens features interacts with the tear meniscus can cause transitions between conformations and the tear volume can exchange tear fluid, for example, with the tear film.

The protrusions can be disposed on the anterior surface of the peripheral portion of the dynamic contact lens outside the optical region so as to not interfere with vision.

The protrusions can be configured to provide a frictional force when dynamically contacted with an eyelid. The frictional force can cause the dynamic contact lens to move on the eye or, for example, can impart a compressive force to the optical portion sufficient to reduce the adhesive capillary forces in the conforming state to release and thereby induce a transition from a conforming configuration to a non-conforming configuration. The protrusions can be disposed symmetrically or asymmetrically around the optical portion. A protrusion can comprise one or more concentric ridges located at various radial distance from the center of the dynamic contact lens. The protrusions can be discrete features located symmetrically about the optical portion, for example, at angles of 120°, 90°, 60°, 45°, or 30°. The protrusions can be disposed outside the optical region of the dynamic contact lens so as to not interfere with vision.

Protrusions are thickened areas in the anterior surface of a lens and are designed to create mechanical forces when there is dynamic contact between the protrusions and the eyelids. A dynamic contact lens can include one or more protrusions. The one or more protrusions can be disposed at a distance from the optical portion of at least about 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, or more. The one or more protrusions can be disposed at a distance from the optical portion of at most about 10 mm, 9.5 mm, 9 mm, 8.5 mm, 8 mm, 7.5 mm, 7 mm, 6.5 mm, 6 mm, 5.5 mm, 5 mm, 4.5 mm, 4 mm, 3.5 mm, 3 mm, 2.5 mm, 2 mm, 1.5 mm, 1 mm, 0.9 mm, 0.8 mm, 0.7 mm, 0.6 mm, 0.5 mm, 0.4 mm, 0.3 mm, 0.2 mm, 0.1 mm, or less. The one or more protrusions can be disposed at a distance from the optical portion that is within a range defined by any two of the preceding values. The one or more protrusions can be disposed at a certain distance from the optical portion such as, for example, within a range from 0.5 mm to 5.5 mm, from 1 mm to 5 mm, from 1.5 mm to 4.5 mm, or from 2 mm to 4 mm from the optical portion. A protrusion may have dimensions of at least about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, or more. A protrusion may have dimensions of at most about 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, or less. A protrusion may have dimensions that are within a range defined by any two of the preceding values. A protrusion can have dimensions, for example, within a range from 0.5 mm to 3 mm, from 1 mm to 3 mm, or from 1 mm to 2 mm. The one or more protrusions can independently have a height from the anterior surface of the dynamic contact lens, for example, from 10 μm to 500 μm, from 50 μm to 450 μm, from 100 μm to 400 μm, or from 150 μm to 350 μm. The one or more protrusions can independently have any suitable cross-sectional profile such as oval-shaped, kidney-shaped, dome-shaped, or oblong-shaped, and the sides can have different slopes.

In embodiments in which a protrusion overlies a reservoir, the protrusion can be designed to be compressible. By compressible in this context is meant that in a configuration in which the reservoir is in a compressed state, the protrusion also moves toward the cornea such that the height of the protrusion above the anterior surface of the dynamic lens is less than that in the compressed state. For example, the protrusion may substantially conform to the curvature of the anterior surface to provide a substantially smooth profile.

In embodiments in which a protrusion overlies a reservoir, cross-sectional thickness at the overlap can be less than the thickness of an adjacent peripheral portion, the same as the thickness of an adjacent peripheral portion, or greater than the thickness of an adjacent peripheral portion.

The one or more protrusions can include surface features that increase friction such as grooves, depressions, and ridges. A groove, depression, or ridge can have dimensions less than the dimensions of a protrusion. For example, a height or depth of a groove, depression, or ridge can be less than 100 μm, less than 75 μm, less than 50 μm, or less than 25 μm. The dimensions of one or more features for increasing friction between an eyelid and the dynamic lens can be selected to facilitate user comfort.

The location and height of the one or more protrusions can be selected such that motion of the eyelids against the protrusions can induce a change in conformation of the optical portion of the dynamic contact lens. The mechanism by which the protrusions can induce a change in conformation can be through changes in capillary forces and/or changes in the internal forces of the dynamic contact lens. The protrusions can be situated such that during down gaze the force of the eyelid against the one or more protrusions causes the optical portion to change conformation.

The one or more protrusions can overly a reservoir such as a tear fluid reservoir. The one or more protrusions may not overly or can partially overly a reservoir such as a tear fluid reservoir.

It should also appreciated that such reservoirs can be compressible or deformable even if there is not overlying protrusion following for example lid pressure. Such compressibility can be achieved by thinning the lens thickness over the reservoir or by increasing the dimension of the reservoir, changing its geometry, changing the general geometry of the lens or by changing the rigidity in the reservoir area such as by using a material having a lower modulus and/or decreasing the thickness of the peripheral portion in the vicinity of the reservoir.

Similar mechanical and fluid dynamics apply to cavities disposed on the peripheral anterior surface and that can be fluidly coupled to the optical tear volume by fenestrations and grooves.

The tear volume can be fluidly coupled to at least one fenestration to facilitate tear fluid movement from and to the space between the lens and the eye. The number of fenestration can be, for example, from 1 to 50, such as from 1 to 20, or from 3 to 10, and can have an internal diameter, for example, from 50 μm to 600 μm, such as from 100 μm to 300 μm.

Dynamic contact lenses provided by the present disclosure can comprise an optical portion, which refers to the region of the dynamic contact lens used for vision and which can assume at least two quasi-stable configurations.

When worn on the eye, the optical portion overlaps with at least a portion of the optical region of the cornea. The dimensions of the optical portion can be less than the dimensions of the optical region, substantially the same as the optical region, or can be less than the dimensions of the optical region of the cornea.

Dynamic contact lenses provided by the present disclosure can comprise a peripheral portion coupled to the optical portion, wherein the peripheral portion is configured to retain the dynamic contact lens on the cornea. The optical portion and the peripheral portion can be coupled at a transition zone. The transition zone can be configured such as dimensioned to facilitate transitions between the conforming and/or non-conforming configurations, control transitions between the conforming and/or non-conforming configurations, stabilize the conforming and/or non-conforming configurations, destabilize the conforming and/or non-conforming configurations, or a combination of any of the foregoing.

For example, the cross-sectional thickness at the transition zone between the peripheral and optical portion can be thinner or thicker than the thickness of the adjacent peripheral and/or optical portion of the dynamic contact lens. For example, in a cross-sectional profile of a dynamic contact lens, the thickness can gradually increase from the peripheral edge of the lens in the peripheral region toward the transition zone with the optical portion. The thickness of the optical portion can be substantially uniform and can be the same as the transition zone thickness, thinner than the transition zone thickness, or thicker than the transition zone thickness. The thickness of the optical portion can increase from the transition zone thickness to the center of the optical portion. The thickness of the optical portion can decrease from the transition zone thickness to the center of the optical portion.

The transition zone can be configured to facilitate maintaining the quasi-stable configurations, to facilitate transitioning between quasi-stable configurations, and/or to control and/or facilitate transport of tear fluid between or among different regions surrounding the dynamic contact lens.

A dynamic contact lens can comprise an optical portion comprising a first material characterized by a first modulus; and a peripheral portion comprising a second material characterized by a second modulus.

The first material and the second material can comprise the same material, or the first material and the second material can comprise different materials.

The first modulus can be greater than the second modulus, the first modulus can be less than the second modulus, or the first modulus can be the same as the second modulus.

The optical portion and the peripheral portion can comprise a single material characterized by a single modulus. As can be appreciated, depending on the thickness of the dynamic lens at a radial distance from the center, the dynamic lens can be characterized by a rigidity that varies with radial distance from the center.

The first modulus may comprise any modulus described herein. The second modulus may comprise any modulus described herein. The first modulus can be within a range, for example, from 0.05 MPa to 100 MPa; and the second modulus can within a range from 0.05 MPa to 100 MPa.

The first modulus can be within a range, for example, from 0.1 MPa to 2 MPa; and the second modulus can be within a range from 0.1 MPa to 2 MPa.

For example, the first modulus and the second modulus can independently be within a range, for example, from 0.05 MPa to 10 MPa, from 0.1 MPa to 8 MPa, from 0.15 MPa to 6 MPa, from 0.2 MPa to 4 MPa, from 0.25 MPa to 3 MPa, from 0.3 MPa to 2 MPa, from 0.3 MPa to 1.5 MPa, for from 0.3 MPa to 1.0 MPa.

The peripheral portion of a dynamic contact lens can comprise a single material characterized by a single modulus. The peripheral portion can comprise different materials having a different modulus. The materials can be different in the sense that the materials have the same fundamental chemistry, such as being silicone hydrogels, but have a different cross-link density and are therefore considered different materials.

Regions of the peripheral portion overlying dynamic features such as dynamic grooves, dynamic fenestrations, or dynamic tear reservoirs can comprise a material having a lower modulus than adjacent regions of the peripheral portion of a contact lens. The use lower modulus material can reduce the rigidity of the region of the peripheral portion. The low modulus material with or without a lower cross-sectional thickness can facilitate the ability of the feature to deform in response to the flow of fluid into, out of, or through the feature. The use of a lower modulus material and therefore less rigid structure can facilitate the ability of the feature to deform in response to interaction with an eyelid. The optical portion can be characterized by a rigidity that is less than a rigidity of the peripheral portion.

Each of the first material, the second material, or the single material can independently comprise a silicone, a hydrogel, a silicone hydrogel, or a combination of any of the foregoing. Any suitable material used to fabricate soft contact lenses can be used. Although the optical portion can be fabricated from a different material than the non-dynamic optical portion, a single basic material can be used to fabricate the dynamic contact lens, however, certain regions can be treated or modified to impart desired mechanical properties. For example, the peripheral portion and the optical portion can comprise the same basic materials, however, certain regions can have a higher cross-linking density or a lower cross-linking density design, for example, to facilitate the ability of the optical portion to exhibit quasi-stable configurations and/or to transition between the quasi-stable configurations in response to force applied to the dynamic contact lens by eyelids.

A dynamic contact lens can comprise a posterior surface; and at least a portion of the posterior surface can comprise a material, a surface treatment, or a combination thereof, selected to control capillary forces between at least a portion of the posterior surface and tear fluid, between the cornea and the tear fluid, between the posterior surface and the cornea, or a combination of any of the foregoing.

The material and/or surface treatment can be selected to provide a surface hydrophobicity, hydrophilicity, polarity, charge, or other attribute that can affect the capillary forces. The properties of the posterior surface can be uniform or can be non-uniform. The surface properties of the posterior surface can be continuous or discontinuous.

Examples of suitable surface treatments include coatings, plasma treatments, and impregnations.

The material itself can be selected to establish a desired surface property.

The properties of the posterior surface of the lens, including the peripheral portion and the optical portion, can be the same or can be different in one or more regions of the posterior surface. For example, one surface property may be desirable to control capillary adhesion of the posterior surface of the optical portion to the cornea, and a different surface property may be desirable, for example in the regions between the tear fluid reservoirs and the optical portion to facilitate exchange of tear fluid.

In a cross-sectional profile, an optical portion can comprise a posterior surface which comprises a gap profile between the posterior surface and the cornea. The gap profile can be characterized by a gap differential, wherein the gap differential is the difference between a center gap height and a peripheral gap height. The gap profile comprises a plurality of gap differentials which decrease with radial distance from the center of the optical portion toward the perimeter transition zone with the peripheral portion. A maximum gap differential can be defined as the difference between a center gap height and a gap height at perimeter of the optical portion.

The conforming configuration can be characterized by a first maximum gap differential; the non-conforming configuration can be characterized by a second maximum gap differential; wherein the second maximum gap differential is greater than the first maximum gap differential.

Dynamic contact lenses provided by the present disclosure can comprise an as-fabricated shape. The as-fabricated shape comprises an optical portion that bulges away from the peripheral base curvature of the peripheral portion from the posterior surface toward the anterior surface.

Dynamic contact lenses may not have an as-fabricated optical portion that bulges anteriorly. An optical portion can have, for example, an anterior surface that is substantially continuous with the anterior surface of the peripheral portion. A tear volume in this configuration can be provided by having the thickness of at least a portion of the optical portion be less than the thickness of the transition zone with the peripheral portion. Such configurations can be useful for providing a lens with negative optical power. Increasing the gap of the optical portion can provide a tear volume that provides a mechanical force to the anterior curvature and thereby changes the optical power of the optical system that can improve near vision such as for near reading.

In one of the at least one non-conforming configurations a dynamic contact lens can comprise the as-fabricated shape.

A dynamic contact lens can comprise a peripheral portion comprising a peripheral posterior surface, wherein the peripheral posterior surface comprises a peripheral base curvature, and the optical portion comprises an optical posterior surface, wherein the optical posterior surface comprises an optical base curvature.

In the conforming configuration the optical posterior base curvature can be substantially the same as the peripheral base curvature.

In a non-conforming configuration, the optical posterior base curvature can deviate from the peripheral base curvature. For example, the curvature of the optical portion can be greater than the peripheral base curvature.

A cornea can be characterized by a corneal curvature. The optical portion of a dynamic contact lens can comprise an optical posterior surface, wherein the optical posterior surface can be characterized by an optical posterior base curvature. In the conforming configuration, the optical posterior base curvature can be substantially the same as the corneal curvature. In a non-conforming configuration, the optical posterior base curvature can deviate from the corneal curvature.

A dynamic contact lens can comprise a peripheral portion comprising a peripheral posterior surface, wherein the peripheral posterior surface comprises a peripheral base curvature, and the optical portion can be characterized by a center sagittal height with respect to the peripheral base curvature.

The optical portion can be characterized by a first center sagittal height with respect to the peripheral base curvature and assume a second configuration characterized by a second center SAG height with respect to the peripheral base curvature, wherein the first center sagittal height and the second center sagittal height are different. The first center sagittal height can be greater than the second center sagittal height or can be less than the second center sagittal height.

The optical portion can be configured to assume a first configuration characterized by a first center gap height with respect to the peripheral base curvature and assume a second configuration characterized by a second center gap height with respect to the peripheral base curvature, wherein the first center gap height and the second center gap height are different. The first center gap height can be greater than the second center gap height or can be less than the second center gap height.

A dynamic contact lens provided by the present disclosure can comprise a peripheral portion comprising a peripheral posterior surface and a peripheral anterior surface, wherein the peripheral posterior surface comprises a peripheral posterior base curvature; and an optical portion comprising an optical posterior surface and an optical anterior surface, wherein at least the optical posterior surface bulges away from the peripheral base curvature toward the optical anterior surface.

A dynamic contact lens can comprise an optical portion comprising an optical posterior surface, wherein the optical posterior surface can be characterized by an optical posterior base curvature; and a peripheral portion coupled to the dynamic optical portion, wherein the peripheral portion comprises a peripheral posterior surface; and the peripheral posterior surface can be characterized by a peripheral posterior base curvature.

In a first configuration the optical posterior base curvature can be substantially the same as the peripheral base curvature; and in a second configuration the optical posterior base curvature can deviate from the peripheral base curvature. In the second configuration the optical posterior base curvature can be less than the peripheral base curvature.

A dynamic contact lens can comprise an optical portion comprising an optical posterior surface, wherein the optical posterior surface comprises an optical posterior base curvature.

In a first configuration the optical posterior base curvature can be substantially the same as a corneal curvature; and in a second configuration the optical posterior base curvature can deviate from the corneal curvature. In the second configuration the optical posterior base curvature can be less than the corneal curvature.

A dynamic contact lens can comprise a peripheral portion comprising a peripheral posterior surface, wherein the peripheral posterior surface can be characterized by a peripheral base curvature; and an optical portion coupled to the peripheral portion, wherein the optical portion comprises a center thickness, and a center sagittal height, a gap height when applied to the cornea, with respect to the peripheral base curvature, or the para-peripheral base curvature adjacent the optical portion.

The optical portion can be configured to assume a first configuration characterized by a first center gap height with respect to the peripheral base curvature and can be configured to assume a second configuration characterized by a second center gap height with respect to the peripheral base curvature.

The first center gap height and the second center gap height can be different.

The first configuration and the second configuration can be quasi-stable.

A dynamic contact lens can comprise an optical portion comprising a posterior surface, wherein the posterior surface comprises an optical posterior base curvature.

In a first configuration the posterior surface of the optical portion can characterized by a first base curvature; and in a second configuration the posterior surface of the optical portion can be characterized by a second base curvature.

The first configuration can be configured to provide a first optical power to an eye having a cornea; and the second configuration can be configured to provide a second optical power to the eye.

The first base curvature can be substantially the same as a corneal curvature.

The dynamic contact lens can further comprise at least one first feature, such as a protrusion, configured to induce a change between the first configuration and the second configuration; and at least one second mechanism configured to induce a change between the second configuration and the first configuration.

In dynamic contact lenses provided by the present disclosure the optical portion can be in the shape of a dome and can have a circular cross section.

Dynamic contact lenses provided by the present disclosure can comprise an optical portion, wherein the as-fabricated optical portion comprises a sagittal height and a center thickness, wherein the center thickness is less than the sagittal height; and a peripheral portion coupled to the optical portion, wherein the peripheral portion is configured to retain the dynamic contact lens on the cornea. With reference to FIG. 1 the sagittal height is the distance between the extension of the curvature of the peripheral portion across the optical portion and the posterior surface of the optical portion at the center axis of the optical portion.

The optical portion can be characterized by a sagittal height, a center thickness, a radial thickness, a posterior surface profile, an anterior surface profile, a diameter, and for spherical profiles, posterior and anterior radii of curvatures.

The as-fabricated sagittal height of the optical portion may be at least about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 225 μm, 230 μm, 235 μm, 240 μm, 245 μm, 250 μm, or more. The as-fabricated sagittal height of the optical portion may be at most about 250 μm, 245 μm, 240 μm, 235 μm, 230 μm, 225 μm, 220 μm, 215 μm, 210 μm, 205 μm, 200 μm, 195 μm, 190 μm, 185 μm, 180 μm, 175 μm, 170 μm, 165 μm, 160 μm, 155 μm, 150 μm, 145 μm, 140 μm, 135 μm, 130 μm, 125 μm, 120 μm, 115 μm, 110 μm, 105 μm, 100 μm, 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 10 μm, 5 μm, or less. The as-fabricated sagittal height of the optical portion may be within a range defined by any two of the preceding values. The as-fabricated sagittal height of the optical portion (110 in FIG. 1) can be within a range, for example, from 5 μm to 300 μm, from 10 μm to 250 μm, from 15 μm to 200 μm, from 20 μm to 150 μm, from 30 μm to 125 μm, or from 40 μm to 100 μm.

In a non-conforming configuration, the gap height may be at least about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 225 μm, 230 μm, 235 μm, 240 μm, 245 μm, 250 μm, or more. In a non-conforming configuration, the gap height of the optical portion may be at most about 250 μm, 245 μm, 240 μm, 235 μm, 230 μm, 225 μm, 220 μm, 215 μm, 210 μm, 205 μm, 200 μm, 195 μm, 190 μm, 185 μm, 180 μm, 175 μm, 170 μm, 165 μm, 160 μm, 155 μm, 150 μm, 145 μm, 140 μm, 135 μm, 130 μm, 125 μm, 120 μm, 115 μm, 110 μm, 105 μm, 100 μm, 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 10 μm, 5 μm, or less. In a non-conforming configuration, the gap height may be within a range defined by any two of the preceding values. In a non-conforming configuration, the gap height (110 in FIG. 1) can be within a range, for example, from 5 μm to 300 μm, from 10 μm to 250 μm, from 15 μm to 200 μm, from 20 μm to 150 μm, from 30 μm to 125 μm, or from 40 μm to 100 μm.

The center thickness of a dynamic contact lens may be at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The center thickness of a dynamic contact lens may be at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. The center thickness of a dynamic contact lens may be within a range defined by any two of the preceding values. The center thickness (112 in FIG. 1) of a dynamic contact lens can be within a range, for example, from 10 μm to 600 μm, from 20 μm to 600 μm, from 30 μm to 600 μm, from 40 μm to 500 μm from 50 μm to 400 μm, from 100 μm to 300 μm, from 150 μm to 200 μm, from 50 μm to 100 μm, from 100 μm to 150 μm, from 150 μm to 200 μm, from 200 μm to 250 μm, or from 250 μm to 300 μm.

The optical portion may be characterized by a diameter of at least about 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, or more. The optical portion may be characterized by a diameter of at most about 10 mm, 9.5 mm, 9 mm, 8.5 mm, 8 mm, 7.5 mm, 7 mm, 6.5 mm, 6 mm, 5.5 mm, 5 mm, 4.5 mm, 4 mm, 3.5 mm, 3 mm, 2.5 mm, 2 mm, 1.5 mm, 1 mm, 0.5 mm, or less. The optical portion may be characterized by a diameter that is within a range defined by any two of the preceding values. The optical portion (115 in FIG. 1) can be characterized by a diameter within a range, for example, from 1 mm 7 mm, from 1.5 mm to 6 mm, from 1.5 mm to 5 mm, from 2 mm to 5 mm, from 2 mm to 4 mm, or from 2.5 mm to 3.5 mm.

The transition zone may have a thickness of at least about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The transition zone may have a thickness of at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, or less. The transition zone may have a thickness that is within a range defined by any two of the preceding values. The transition zone (108 in FIG. 1) can have a thickness within a range, for example, from 10 μm to 600 μm, from 20 μm to 600 μm, from 30 μm to 600 μm, from 40 μm to 500 μm from 50 μm to 400 μm, from 100 μm to 300 μm, from 150 μm to 200 μm, from 50 μm to 100 μm, from 100 μm to 150 μm, from 150 μm to 200 μm, from 200 μm to 250 μm, or from 250 μm to 300 μm.

The optical portion can have a spherical profile and the radius of curvature of the posterior surface and/or the anterior surface can be, for example, within a range from 5 mm to 10 mm, from 4 mm to 9 mm, from 3 mm to 8 mm, from 5 mm to 6 mm, from 6 mm to 7 mm, from 7 mm to 8 mm, from 8 mm to 9 mm, from 9 mm to 10 mm, or from 10 mm to 11 mm.

The optical portion of a dynamic contact lens can comprise a posterior surface and an anterior surface.

As fabricated, the shape of the optical portion including the posterior and anterior surfaces can comprise an outward bulge or dome in which the optical portion extends from the posterior to anterior direction and away from the profile of the peripheral base curvature.

In dynamic contact lenses provided by the present disclosure the optical portion can be configured to assume two or more configurations in which each of the two or more configurations do not conform to the surface of the cornea. Thus, a dynamic contact lens can comprise an optical portion, wherein the optical portion comprises at least one first non-conforming configuration configured to provide a first optical power to an eye having a cornea; and at least one second non-conforming configuration configured to provide a second optical power to the eye, wherein the second optical power is different than the first optical power; at least one first physical feature configured to induce a change between the first non-conforming configuration and the at least one second non-conforming configuration; and at least one second physical feature configured to induce a change between the at least one second non-conforming configuration and the at least one first non-conforming configuration.

A volume of an optical tear volume can be, for example, within a range from 0.001 μL to 0.01 μL, from 0.001 μL to 0.1 μL, from 0.01 μL to 10 μL, from 0.02 μL to 8 μL, from 0.05 μL to 7 μL, from 0.1 μL to 6 μL, from 0.1 μL to 5 μL, from 0.5 μL to 4 μL, or within a range from 1 μL to 3 μL.

The peripheral portion can have a diameter, for example, within a range from 8 mm to 17 mm, from 8.5 mm to 16.5 mm, from 9 mm to 16 mm, or from 9.5 mm to 15.5 mm.

The peripheral portion can be characterized by base curvature, i.e., the curvature of the anterior surface within a range, for example, from 7 mm to 10 mm, from 7.2 mm to 9.8 mm, from 7.4 mm to 9.6 mm, from 7.6 mm to 9.4 mm, from 7.8 mm to 9.2 mm, or from 8 mm to 9 mm.

In certain dynamic contact lenses provided by the present disclosure, the optical portion can be configured to facilitate dynamically changing between configuration when applied to the eye. For example, the optical portion can change configuration during dynamic contact with an eyelid induced or when one of the lens features interacts with the tear meniscus, for example, by a change in gaze angle, by normal blinking, by intentional blinking, by holding the eyelids closed, or by squeezing the eyelids against the eye.

The posterior and anterior surfaces of the optical portion can independently have a spherical profile or a non-spherical profile. For example, the thickness of the optical portion can be substantially constant throughout the profile, can be thinner toward the center than toward the transition zone, or can be thicker toward the center than toward the transition zone.

Dynamic contact lenses can have an optical portion comprising a posterior surface characterized by a first radius of curvature; and a peripheral portion characterized by at least one second radius of curvature; wherein the first radius of curvature is less than the second radius of curvature. In other words, the optical portion extends anteriorly from the peripheral base curvature.

The optical portion of a dynamic contact lens comprises a thickness. The thickness of the optical portion can comprise a center thickness, which refers to the thickness of the optical portion at the physical center of the optical portion, and a plurality of radial thicknesses that span the segment of the optical portion from the center to the transition zone of the optical portion with the peripheral portion.

The thickness of the optical portion can be substantially uniform across the profile. In certain lenses, the thickness can vary or be non-uniform across the profile. For example, the center thickness can be greater than each of the plurality of radial thicknesses. The thickness of the optical portion can be radially symmetric about the center axis of the optical portion.

The thickness of the optical portion may not be uniform across the profile. The thickness can be greater toward the center or less toward the center compared to the periphery. The thickness of the optical portion can also vary across the profile.

The optical portion and the optical portion can be aligned with the optical axis of the dynamic contact lens. The optical axis of the dynamic contact lens refers to the center axis of the lens. In some embodiments, the optical portion is not aligned with the optical axis of the lens.

The optical region can be characterized by a diameter within a range, for example, from 1 mm to 8 mm, from 2 mm to 7 mm, or from 3 mm to 6 mm.

The optical portion and the peripheral portion of a dynamic contact lens provided by the present disclosure can comprise a silicone, a hydrogel, or a silicone hydrogel. Any suitable soft contact lens material can be used.

The optical portion and the peripheral portion of a dynamic contact lens can comprise the same material. The optical portion and the peripheral portion can comprise different materials characterized, for example, by different physical and/or mechanical properties. The optical portion and the peripheral portion can be characterized by materials having a different modulus, and the portions can exhibit different rigidities.

The optical portion and the peripheral portion can also be characterized by a rigidity. The cross-sectional rigidity is proportional to the material modulus time the cube of the cross-sectional thickness. As can be appreciated, when the peripheral portion comprises a single material, the cross-sectional rigidity increases as the thickness increases from the edge of the peripheral portion toward the transition zone with the optical portion.

Dynamic contact lenses provided by the present disclosure can comprise a deformable optical portion and a peripheral portion coupled to the deformable optical portion. The optical portion can be configured to deform to accommodate a depth of vision. The peripheral portion can be configured to retain the dynamic contact lens on a cornea.

When applied to the eye, lenticular volumes between the posterior surface of the optical portion and the anterior surface of the cornea can fill with tear fluid to form a tear volume. In a dynamic contact lens, the optical portion is configured to change shape depending on the distance of vision. The change in configuration of the optical portion provides a tear volume. The configuration of the optical portion can change continuously or can assume discrete configurations. It should be appreciated that a dynamic contact lens having an optical portion can be fabricated having a dome extending outward (posterior to anterior direction) from the curvature of the peripheral portion. It should also appreciated that when the fabricated lens with a dome extending outward is worn by the user, the dome can extend outward less than as-fabricated. In other words, when applied to the cornea, the dynamic contact lens can stretch outwardly.

The first and second configurations correspond to different optical powers imparted by the optical anterior surface. The first configuration can be appropriate for distance vision and the second configuration can be appropriate for near vision. The first configuration can be appropriate for near vision and the second configuration can be appropriate for distance vision.

An objective of the optical portion is to facilitate changing the optical power of the optical portion in response to the viewing distance of an eye. For example, in a first configuration suitable for distance vision the optical portion will be disposed proximate the anterior corneal surface, and for near vision the optical portion will extend away from the cornea to form a tear volume.

In dynamic contact lenses, the optical power of the optical portion does not change when the configuration of the optical portion changes. For example, the thickness of the optical portion and the relative cross-sectional profiles of the posterior and anterior surfaces of the dynamic optical portion do not change as the optical portion assumes different configurations. Therefore, the optical power of the lens itself does not change (i.e the relationship of the anterior curve to the posterior curve and the refractive index remain constant). The shape of the peripheral portion does not change appreciably when the configuration of the optical portion changes. The peripheral portion can be configured to retain the dynamic contact lens on the cornea, keeping the dynamic contact lens centered on the optical region of the cornea and minimizing translation of the dynamic contact lens on the cornea. For example, translation of the dynamic contact lens on the cornea can be less than ±1.5 mm, less than ±1.0 mm, or less than ±0.5 mm.

In the different configurations, the center thickness of the optical portion and the radial thicknesses of the optical portion may not appreciably change. For example, the optical portion can comprise a plurality of radial thicknesses, and the plurality of radial thicknesses in a first configuration is substantially the same as the corresponding radial thicknesses in a second configuration.

The uniform profile of the optical portion with changing configurations can also be considered in terms of the curvature. In certain dynamic contact lenses, the optical portion will not have an optical power and the posterior and anterior surfaces of the optical portion will have spherical profiles characterized by the same radius of curvature. The radius of curvature can be defined by the diameter of the optical portion, the thickness of the peripheral portion at the transition zone with the optical portion, and the gap height.

In certain dynamic contact lenses, the optical portion can comprise a posterior surface comprising a first radius of curvature, the optical portion can comprise an anterior surface comprising a second radius of curvature, and a ratio of the first radius of curvature to the second radius of curvature in the first configuration is the same as the ratio in the at least one second configuration.

In certain dynamic contact lenses, the optical portion can be characterized by a plurality of radial thicknesses, wherein each of the plurality of radial thicknesses is substantially the same throughout the range of gap heights accessible to the optical portion.

The configuration of the optical portion can be configured to change upon application of a force applied to the dynamic contact lens by eyelids. The force can be applied to the peripheral portion, a region of the peripheral portion and/or to the optical portion.

The tear meniscus can also serve as a major source of tear fluids and can act as a driving force for activation of the tear volume. For example, during near viewing and/or gazing down, a fenestration can become fluidly coupled to the tear meniscus and a source of tear fluid can become available to fill the optical tear volume and enable the optical zone to transition to a near vision configuration by forming a tear volume.

Thus, a dynamic contact lens can be configured such that during primary gaze there is no fluid connection between the tear meniscus and the optical portion, while during downgaze the dynamic contact lens can be configured such that a fenestration and/or other fluid transport element, moves downward to become fluidly coupled to the tear meniscus. Using specific dimensions for the fenestration and other elements such as posterior grooves, anterior grooves and/or depressions, and the for optical portion of the lens, passive forces such as capillary forces and capillary valve forces, and active forces such as a pumping force generated by the optical portion, tear fluid can flow from the tear meniscus into the optical tear volume to create a tear volume.

The eyelid force can be applied by changing a gaze angle such as gazing forward for distance vision or by gazing downward such as for near vision. The eyelid force can be applied by normal blinking, or by intentional blinking Intentional blinking can involve, hold the eyelids closed for a period of time, squeezing the eyelids closed for a period of time, and/or repeating either of the foregoing multiple times.

The eyelid forces can be used to transition the optical portion from one configuration to another and/or to accelerate the transition from one configuration to another.

As the optical portion changes configuration caused by force applied by the eyelids, the optical power of the anterior optical surface can change.

As fabricated, the optical portion of a dynamic contact lens extends anteriorly to form a dome with respect to the extended profile of the peripheral portion of the dynamic contact lens.

In a configuration in which the optical portion is proximate to the anterior surface of the cornea, the optical portion can be held in this quasi-stable configuration by a combination of adhesive and cohesive capillary forces. As the thickness of the layer of tear film decreases the adhesive forces between the posterior surface of the optical portion and the anterior surface of the cornea will become greater than the cohesive forces of the tear fluid, thereby causing the optical portion to assume a quasi-stable configuration in which the optical portion substantially conforms to the surface of the cornea.

The transition of the optical portion between or among the two or more configurations induced by eyelid forces can be facilitated using various methods and features.

In certain methods the capillary forces holding the optical portion against the cornea can be broken by increasing the separation between the two surfaces. This can be accomplished, for example, by pushing tear fluid between the surfaces thereby reducing the adhesive force and causing the posterior surface of the optical portion to release. Depending on the construction, upon release the optical portion can assume a fully extended dome-shaped configuration and tear fluid can be pulled from the transition zone between the posterior surface of the peripheral portion and the cornea to fill the tear volume with tear fluid. Alternatively, or in conjunction with, repeated blinking can be used to facilitate the movement of tear fluid into and/or from the tear volume. The blinking can comprise intentional blinking whereby a user can achieve a desired vision correction without the optical portion being fully extended.

In certain methods frictional forces imparted by an eyelid to the peripheral portion can be used to change the configuration of the optical portion and hence the optical power of the anterior optical surface. In such methods an eyelid can grab the peripheral portion and physically squeeze the dynamic contact lens toward the center to impart a force sufficient to overcome the capillary forces holding the optical portion against the cornea and thereby cause the posterior surface optical portion to release and provide a tear volume. Examples of physical lens features that could be used to facilitate the ability of an eyelid to impart a mechanical force include protrusions such as ridges on the anterior surface of the peripheral portion of the dynamic contact lens, thickening in the peripheral portion, features to increase friction between the edge of the peripheral portion and the conjunctiva, and use of multiple curvatures in the peripheral portion.

The optical portion in an extended configuration can be brought against the corneal surface by intentional blinking.

In addition to or as an alternative to the above methods, changing the configuration of the optical portion can be facilitated by manipulating the flow of tear fluid to and from tear fluid reservoirs.

A dynamic contact lens provided by the present disclosure can comprise a plurality of cavities disposed on the posterior surface of the peripheral portion. It can be desirable that the cavities are outside the optical region of the lens so as not to interfere with vision.

A dynamic contact lens can be fabricated such that the posterior surface of the peripheral portion comprises one or more cavities.

The one or more cavities can be configured to provide one or more tear fluid reservoirs when the dynamic contact lens is applied to the cornea.

The one or more cavities can be configured to provide one or more compressible tear fluid reservoirs when the dynamic contact lens is applied to the cornea. The thickness of the peripheral portion between the cavity and the anterior surface of the peripheral portion can be sufficiently thin such that a force applied by an eyelid can compress the cavity. The eyelid force can be imparted by blinking, intentional blinking, or by the motion of an eyelid moving over the cavity.

The cavities can be disposed and configured in any suitable manner to facilitate the transition of the optical portion between two or more configurations.

For example, the one or more cavities can be disposed symmetrically about the optical portion. The one or more cavities can be disposed asymmetrically about the optical portion.

The one or more cavities can comprise one or more concentric rings, one or more grooves, one or more wedge-shaped cavities, and/or one or more rounded cavities.

The cavities can be continuous around the optical portion or can comprise a plurality of separate cavities. The cavities can be elongated such as oblong or wedge-shaped where the long axis points toward the center of the lens. The separate cavities can be fluidly coupled with grooves to facilitate filling and flow of tear fluid between the cavities and/or between the cavities and the optical portion.

For example, a separate cavity can have a width within a range from 0.1 mm to 5 mm, a length within a range from 0.1 mm to 5 mm, and a depth within a range from 10 μm to 200 μm.

The cavities can be continuous, semi-continuous or separated. A continuous cavity refers to a single cavity disposed around the optical portion. An example of a continuous cavity is a concentric ring or a plurality of concentric rings. The concentric rings can have any suitable cross-sectional shape. For example, the cross-sectional shape can be rounded, oval, square, rectangular, triangular, and/or angled. Multiple concentric rings can be fluidly coupled by one or more fluid grooves.

An example of separated fluid cavities is multiple cavities disposed about the optical portion of the dynamic contact lens. The multiple cavities can be disposed symmetrically about the optical portion such as separated by 45° or can be disposed at intervals around the optical portion. For example, groups of cavities can be disposed about the optical portion, for example, at 120°, 90°, 60°, 45°, or 30° intervals or any other suitable interval. The separated cavities can have any suitable dimension and cross-sectional shape. For example, the separated cavities can have a hemispherical or triangular cross-sectional shape. The cavities can be oval, oblong, cylindrical, circular or any other suitable cross-sectional shape. The cavities can be symmetrical or can be characterized by a length different than the width.

The one or more cavities can be disposed at a certain distance from the optical portion such as, for example, within a range from 0.5 mm to 5.5 mm, from 1 mm to 5 mm, from 1.5 mm to 4.5 mm, or from 2 mm to 4 mm from the optical portion. A cavity can have dimensions, for example, within a range from 0.5 mm to 3 mm, from 1 mm to 3 mm, or from 1 mm to 2 mm. The one or more cavities can independently have a height from the anterior surface of the dynamic contact lens, for example, from 10 μm to 500 μm, from 50 μm to 450 μm, from 100 μm to 400 μm, or from 150 μm to 350 μm. The one or more cavities can independently have any suitable cross-sectional profile such as oval-shaped, kidney-shaped, dome-shaped, or oblong-shaped, and the sides can have different slopes.

Semi-continuous cavities refer to separated cavities that are fluidly coupled by grooves formed in the posterior surface of a dynamic contact lens. The grooves can allow tear fluid to flow between adjacent tear fluid reservoirs.

When disposed on a cornea, the cavities can fill with tear fluid to form tear fluid reservoirs.

When compressed by motion of an eyelid or dynamic contact by an eyelid with a change in gaze angle, tear fluid can be pushed toward the optical portion of the dynamic contact lens to break the capillary forces holding the optical portion against the cornea and/or to cause the SAG height to increase. The tear fluid reservoirs can provide a source of tear fluid for filling the tear volume, thereby facilitating a faster response in changing from one configuration to another.

When eyelid pressure is removed, the reservoirs can expand and act to pull tear fluid from the tear volume to fill the reservoirs with tear fluid, effectively pulling the optical portion toward the cornea. The cavities and resulting tear fluid reservoirs can serve to push and pull tear fluid to and from the tear volume. The cavities can serve to modify the internal mechanical properties of a dynamic contact lens to facilitate the transition of the optical portion between quasi-stable configurations.

Symmetrically disposing the cavities and tear fluid reservoirs about the optical portion can render the function of the dynamic contact lens independent of orientation on the eye. Having the dynamic contact lens be rotationally symmetric can facilitate a user's ability to wear the dynamic contact lens.

This push/pull action of the compressible cavities to facilitate the transition of the optical portion from one configuration to another can serve as the only mechanism for changing configuration or can be augmented by intentional blinking. For example, intentionally blinking can help to stabilize the configuration in which the optical portion is proximate to the corneal surface, for example, by expelling tear fluid from or by thinning the tear fluid layer between the optical portion and the cornea.

Dynamic contact lenses provided by the present disclosure can have an optical portion but not include a mechanism for transitioning between configurations. A dynamic contact lens can have an as-fabricated shape in which the optical portion bulges anteriorly from the base curvature of the posterior surface of the peripheral portion. When applied to a cornea, the optical portion forms a tear volume. However, unlike a tear volume, in this embodiment, the dynamic contact lens can produce a tear volume that does not change configuration with a change in eyelid pressure on the lens. In certain embodiments, of the contact lenses the optical portion can be configured to resist deformation. With reference to a contact lens having an optical portion configured to assume conforming and at least one or more conforming configuration, or multiple non-conforming configurations a contact lens having a static tear volume will assume a single non-conforming configuration when placed on the cornea. The contact lens, optical portion, and peripheral portion of a lens configured to have a static tear volume can be dimensioned as for a contact lens in which the optical portion is configured to assume multiple configurations. Contact lenses having a static tear volume can be suitable for correcting vision of an irregular cornea, treating astigmatism, and for corneal wound healing.

Dynamic contact lenses provided by the present disclosure can comprise one or more fenestrations.

The one or more fenestrations can be disposed in the peripheral portion of the lens and outside the optical region so as not to interfere with vision.

The one or more fenestrations can extend through the thickness of the peripheral portion can fluidly couple the anterior surface and the posterior surface of the peripheral portion. The fenestrations can facilitate the flow of tear fluid to the tear film adjacent the epithelium, and depending on the lens configuration, can facilitate the flow of tear fluid to and from the tear volume and/or can facilitate the exchange of tear fluid along the epithelium to promote eye health.

The one or more fenestrations can be fluidly coupled to one or more cavities. The fenestrations can allow tear fluid to flow from the anterior surface of the dynamic contact lens into one or more cavities, which can facilitate the transition of the optical portion between different configurations.

Fenestrations can be fluidly coupled to grooves in the posterior surface of the dynamic contact lens. The grooves can extend from the peripheral region of the lens to the optical portion. Grooves can also fluidly couple fluid cavities which may or may not be fluidly coupled to the optical portion of the dynamic contact lens.

The tear meniscus is a source of tear fluid for exchange with the tear volume.

The tear meniscus can be accessed by fluidly coupling one or more fenestrations with the upper and/or lower tear menisci.

Fenestrations having diameter from 25 μm to 500 μm are relatively small and bringing the opening of a fenestration into contact with the shallow tear meniscus can be difficult. To facilitate the ability of a fenestration to fluidly couple with the tear meniscus, the anterior orifice of a fenestration can be disposed within a depression or cavity in the anterior surface of the dynamic contact lens. The large depression, compared to the diameter of a fenestration, can facilitate the ability of the anterior orifice of the fenestration to fluidly couple with the tear meniscus. The depression can have a diameter, for example, from 0.5 mm to 4 mm, such as from 1 mm to 3 mm. A depression can have a depth, for example, from 3 μm to 150 μm. The depression can have any suitable cross-sectional profile such as, for example, round, oval, slit, oblong, or can have an irregular contour. The edges of the depression can be smoothed or chamfered to facilitate fluid coupling to the fenestration and/or to improve comfort.

For example, FIGS. 15A-15H show views of a dynamic contact lens having depressions and fenestrations within the depressions disposed in the second peripheral portion near the transition zone. FIGS. 15A and 15B show views of the anterior surface and a cross-sectional view, respectively, of the dynamic contact lens. The dynamic contact lens shown in FIGS. 15A and 15B includes first peripheral portion 1501, second peripheral portion 1502, optical portion 1503, transition zone 1506, fenestration 1504 within depression 1507, and posterior groove 1505. FIG. 15C shows a magnified cross-sectional view illustrating the depression 1507 and fenestration 1504, which are coupled to a groove 1505 in the posterior surface of the contact lens. FIG. 15C shows a depression 1507 and fenestration 1504 in peripheral portion 1502 coupled to posterior groove 1505. FIG. 15D shows a magnified top view of the elements shown in FIG. 15C including peripheral posterior surface 1502, depression 1507 and fenestration 1504. FIG. 15E shows a view of the posterior surface of a dynamic contact lens including first peripheral portion 1501, second peripheral portion 1502, optical portion 1503, and depression 1507 with a fenestration 1504. FIG. 15F shows the anterior surface of the dynamic contact lens shown in FIG. 15E including first peripheral portion 1501, second peripheral portion 1502, optical portion 1503, and depression 1507 with a fenestration 1504. As shown in FIGS. 15D and 15F, the depression and fenestration are located in proximity to the transition zone 1506 and to the optical portion 1503. FIG. 15G shows a view of the posterior surface of a dynamic contact lens including first peripheral portion 1501, second peripheral portion 1502, optical portion 1503, and groove 1505 with a fenestration 1004. Groove 1505 extends from the fenestration into the optical portion. 1503. FIG. 15H shows the anterior surface of the dynamic contact lens shown in FIG. 15G including first peripheral portion 1501, second peripheral portion 1502, optical portion 1503, and depression 1507 with a fenestration 1504.

Alternatively, or in addition to a depression, a fenestration can be fluidly coupled to grooves on the anterior surface of the peripheral portion configured to draw fluid from the tear meniscus toward and into the fenestration by capillary forces. Examples of these structures are shown in FIGS. 16A-16C. FIGS. 16A-16C show side, perspective, and cross-sectional views, respectively, of a dynamic contact lens having a first peripheral portion 1601, a second peripheral portion 1602, an optical portion 1603, and a depression 1604 in the anterior surface of the second peripheral portion 1602 with a fenestration 1605 in the bottom of the depression 1604. As shown in FIG. 16B, on the posterior surface, a groove 1606 is coupled to the fenestration 1605 and extends from the second peripheral portion 1602 into the optical portion 1603. A cross-sectional view of the dynamic contact lens is shown in FIG. 16C, and in addition the elements shown in FIGS. 16A-16B, shows that the posterior groove 1606 narrows toward the optical portion 1603 and is fluidly coupled to optical tear volume 1607.

To enhance the ability to access the tear meniscus, multiple fenestrations located at different radial distances from the physical or optical center of the dynamic contact lens can be used. For example, a fenestration having a dimension of 250 μm can be located at a radial distance of 3.5 mm, and a fenestration having a dimension of 300 μm can be located at a radial distance of 4 mm. Other fenestration dimensions, radial distances, and number of fenestrations may be used. The multiple fenestrations may be on the same meridian or on different meridians. If used, depressions surrounding the anterior orifice of a fenestration can be different or the same for different fenestrations. For example, a depression can have a maximum dimension, for example, from 10 μm to 5 mm, such as from 50 μm to 4 mm, from 100 μm to 3 mm, from 200 μm to 2 mm, or from 500 μm to 1.5 mm. A depression can have a depth, for example from, 3 μm to 600 μm, from 10 μm to 500 μm, from 100 μm to 400 μm, from or from 150 μm to 300 μm. A depression can have any suitable shape, such as, for example, oval, round, oblong, triangle, or rectangular.

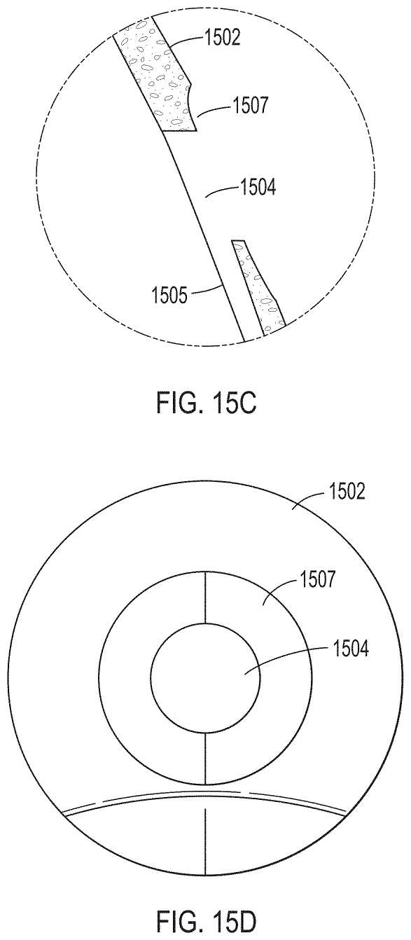

An example of multiple fenestrations for coupling to a tear meniscus is shown in FIGS. 17A-17D. FIGS. 17A-17D show dynamic contact lenses having a first peripheral portion 1701, a second peripheral portion 1702, and an optical portion 1703. Fenestrations 1704 are radially disposed around the optical portion at various radial distances from the center of the optical portion 1703. FIGS. 17A and 17B show anterior and posterior views, respectively, of a dynamic contact lens having 24 fenestrations disposed in 12 radial segments of two fenestrations each. As shown in FIG. 17B, the fenestrations 1704 are coupled to posterior grooves 1705 that extend from the second peripheral portion 1702 into the optical portion 1703. FIGS. 17C and 17D show anterior and posterior views, respectively, of a dynamic contact lens having 36 fenestrations disposed in 12 radial segments of three fenestrations each, where the fenestrations 1704 are disposed at various radial distances from the center of the optical portion 1703. As shown in FIG. 17D, each of the fenestrations is coupled to a radial groove 1705 that extends from the second peripheral portion 1702 into the optical portion 1703.

To facilitate coupling a posterior groove with a tear meniscus, an elongated fenestration can be used. An elongated fenestration extent at an angle with respect to the surfaces of the dynamic contact lens, rather than be substantially orthogonal to the surface of the dynamic contact lens. An elongated fenestration can have a length, for example, from 0.6 mm to 5 mm, from 0.8 mm to 4 mm, from 1 mm to 3 mm, or from 1.5 mm to 2.5 mm.

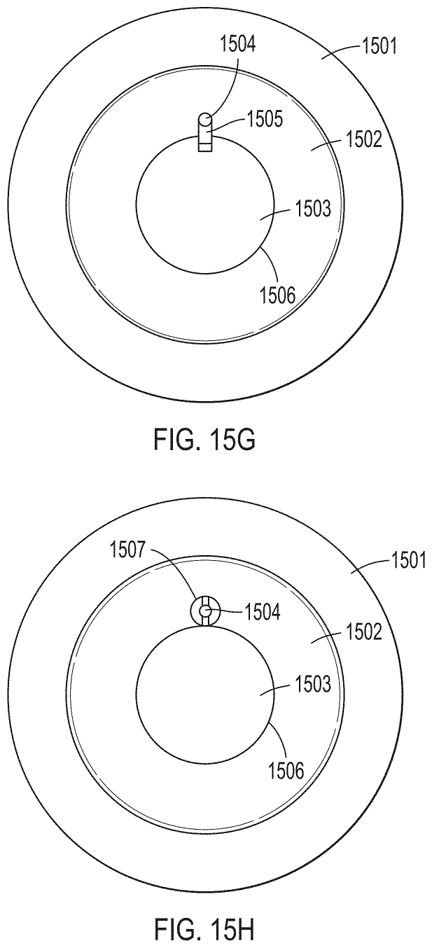

FIGS. 18A-18C and 19A-19C show examples of anterior grooves that extend radially from the periphery of the dynamic contact lens toward the optical portion and are connected to a fenestration, which in turn is connected to a posterior groove. When in contact with the tear meniscus, tear fluid can be drawn from the tear meniscus, through the anterior groove, through the fenestration, through the posterior groove and into the optical tear volume by capillary and/or a combination of forces. FIGS. 18A-18C show first peripheral portion 1801, second peripheral portion 1802, optical portion 1803, radial anterior groove 1805, and fenestration 1805. FIG. 18B shows fenestration 1804 connected to posterior groove 1806 that extends from the fenestration 1804 into the optical zone 1803. FIG. 18C shows a cross-sectional view including anterior groove 1805 connected to posterior groove 1806 by fenestration 1804. Posterior groove 1806 narrows at the transition zone interface with the optical portion 1803, and couples the anterior groove 1805 to the optical tear volume 1807. Anterior channel 1805 can be configured to fluidly couple to a tear meniscus of the eye such as during downward gaze.

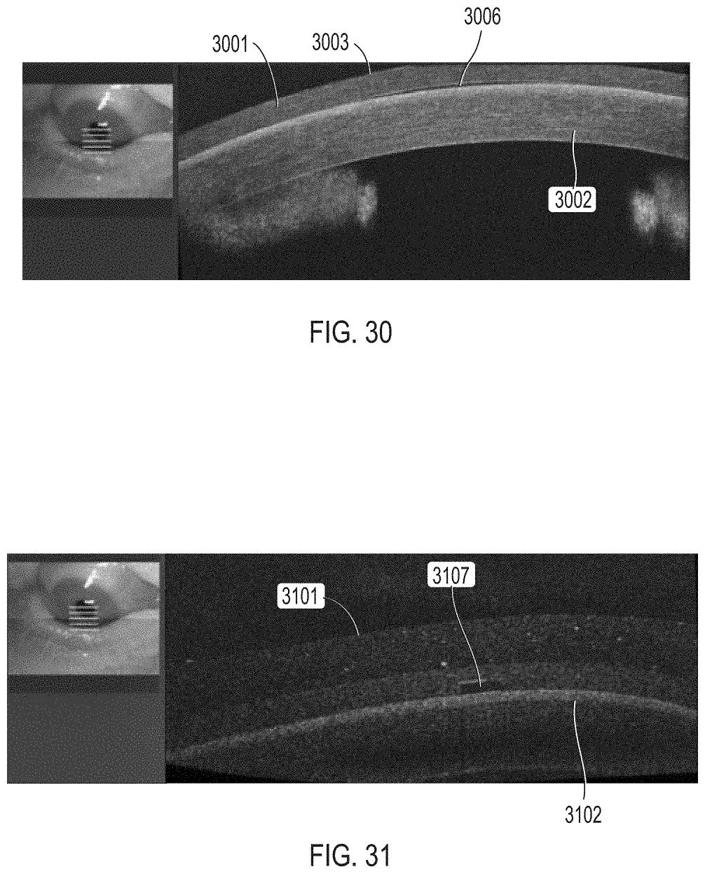

FIG. 32 shows anterior grooves extending from the peripheral portion to the optical portion. The anterior grooves have various lengths to facilitate fluid coupling to a tear meniscus.

FIGS. 19A-19C show views of the anterior surface, posterior surface, and cross-section, respectively, of an example of a dynamic contact lens. As shown in FIG. 19A, the lens includes first peripheral portion 1901, second peripheral portion 1902, optical portion 1903, and cavities 1904 in the anterior surface of the second peripheral portion 1902 with a fenestration 1905 in each of the cavities 1904. As shown in FIG. 19B, on the posterior surface, a groove 1906 extends from the fenestration 1905 into the optical portion 1903. As shown in FIG. 19C, the cavity 1904 is coupled to the tear volume 1907 by the fenestration 1905 and the posterior groove 1906. Anterior cavity 1904 can be configured to fluidly couple to a tear meniscus of the eye such as during downward gaze.

The tear meniscus can theoretically provide sufficient tear fluid to fill the optical tear volume.

The calculated relationship between the optical tear volume and the optical power of the tear volume can be determined by the diameter of the optical portion and the pre-fabricated sagittal height, which represents the largest tear volume, is shown in Table 1. As demonsrated in Table 1, a tear fluid in a meniscus which has a typical volume of 0.1 μL to 1 μL is sufficient to fill the optical tear volume for correcting vision up to 3D, for an optical portion diameter from 3 mm to 7 mm.

TABLE 1
Relationship between optical tear volume and optical power.

| Optical Portion | | | |
|---|---|---|---|
| Diameter | Optical Power | Volume | SAG |
| (mm) | (D) | (mm³) | (μm) |
| 3.00 | 1.50 | 0.02 | 5.26 |
| 3.00 | 2.00 | 0.03 | 7.02 |
| 3.00 | 2.50 | 0.03 | 8.78 |
| 3.00 | 3.00 | 0.04 | 10.54 |
| 3.00 | 3.50 | 0.04 | 12.30 |
| 3.00 | 4.00 | 0.05 | 14.06 |
| 3.00 | 4.50 | 0.06 | 15.82 |
| 3.00 | 5.00 | 0.06 | 17.59 |
| 3.5 | 1.50 | 0.04 | 7.24 |
| 3.5 | 2.00 | 0.05 | 9.66 |
| 3.5 | 2.50 | 0.06 | 12.08 |
| 3.5 | 3.00 | 0.07 | 14.50 |
| 3.5 | 3.50 | 0.08 | 16.93 |
| 3.5 | 4.00 | 0.09 | 19.35 |
| 3.5 | 4.50 | 0.11 | 21.78 |
| 3.5 | 5.00 | 0.12 | 24.22 |
| 4 | 1.50 | 0.06 | 9.58 |
| 4 | 2.00 | 0.08 | 12.78 |
| 4 | 2.50 | 0.10 | 15.98 |
| 4 | 3.00 | 0.12 | 19.19 |
| 4 | 3.50 | 0.14 | 22.40 |
| 4 | 4.00 | 0.16 | 25.62 |
| 4 | 4.50 | 0.18 | 28.84 |
| 4 | 5.00 | 0.21 | 32.06 |
| 4.5 | 1.50 | 0.10 | 12.29 |
| 4.5 | 2.00 | 0.13 | 16.41 |
| 4.5 | 2.50 | 0.17 | 20.52 |
| 4.5 | 3.00 | 0.20 | 24.65 |
| 4.5 | 3.50 | 0.23 | 28.78 |
| 4.5 | 4.00 | 0.27 | 32.92 |
| 4.5 | 4.50 | 0.30 | 37.07 |
| 4.5 | 5.00 | 0.34 | 41.22 |
| 5 | 1.50 | 0.16 | 15.43 |
| 5 | 2.00 | 0.21 | 20.59 |
| 5 | 2.50 | 0.26 | 25.77 |
| 5 | 3.00 | 0.31 | 30.95 |
| 5 | 3.50 | 0.37 | 36.15 |
| 5 | 4.00 | 0.42 | 41.36 |
| 5 | 4.50 | 0.47 | 46.58 |
| 5 | 5.00 | 0.52 | 51.81 |
| 5.5 | 1.50 | 0.23 | 19.02 |
| 5.5 | 2.00 | 0.31 | 25.39 |
| 5.5 | 2.50 | 0.39 | 31.78 |
| 5.5 | 3.00 | 0.47 | 38.18 |
| 5.5 | 3.50 | 0.55 | 44.60 |
| 5.5 | 4.00 | 0.63 | 51.04 |
| 5.5 | 4.50 | 0.71 | 57.50 |
| 5.5 | 5.00 | 0.79 | 63.98 |
| 6 | 1.50 | 0.34 | 23.11 |
| 6 | 2.00 | 0.45 | 30.86 |
| 6 | 2.50 | 0.57 | 38.63 |
| 6 | 3.00 | 0.68 | 46.43 |
| 6 | 3.50 | 0.80 | 54.26 |
| 6 | 4.00 | 0.92 | 62.11 |
| 6 | 4.50 | 1.03 | 69.99 |
| 6 | 5.00 | 1.15 | 77.90 |
| 6.5 | 1.50 | 0.48 | 27.75 |
| 6.5 | 2.00 | 0.65 | 37.07 |
| 6.5 | 2.50 | 0.81 | 46.43 |
| 6.5 | 3.00 | 0.97 | 55.83 |
| 6.5 | 3.50 | 1.14 | 65.26 |
| 6.5 | 4.00 | 1.30 | 74.73 |
| 6.5 | 4.50 | 1.47 | 84.24 |
| 6.5 | 5.00 | 1.64 | 93.79 |
| 7 | 1.50 | 0.67 | 33.03 |
| 7 | 2.00 | 0.90 | 44.14 |
| 7 | 2.50 | 1.13 | 55.30 |
| 7 | 3.00 | 1.36 | 66.51 |
| 7 | 3.50 | 1.59 | 77.78 |
| 7 | 4.00 | 1.82 | 89.10 |
| 7 | 4.50 | 2.05 | 100.48 |
| 7 | 5.00 | 2.29 | 111.92 |

The posterior surface of the optical portion, the posterior surface of the peripheral portion, or the posterior surfaces of both the optical portion and the peripheral portion can comprise a surface treatment.

The surface treatment can be configured to control, modify, and/or select the adhesive and cohesive force of tear fluid to the posterior surface of the optical portion, the posterior surface of the peripheral portion, or the posterior surfaces of both the optical portion and the peripheral portion.

A surface treatment may be applied to all or to a portion of the inner posterior surface and/or the peripheral posterior surface of a dynamic contact lens.

In dynamic contact lenses comprising cavities, a surface treatment may be applied to the walls of the cavities and/or to grooves extending from the cavities.

A surface treatment can comprise, for example a coating, a thin film, a chemical treatment, a plasma treatment or a combination of any of the foregoing.

A surface treatment can be selected to modify the hydrophobicity/hydrophilicity of the posterior surface of the optical portion, the posterior surface of the peripheral portion or the posterior surfaces of both the optical portion and the peripheral portion.

A surface treatment can be selected to control and/or to tailor the capillary forces between the posterior surface of the optical portion and the cornea.

A surface treatment can be selected to control and/or facilitate the flow of tear fluid to and from the optical tear volume.

A posterior surface of a dynamic contact lens can comprise a material selected to control the hydrophilicity/hydrophobicity of the posterior surface. A posterior surface can comprise a material selected to control the charge of the posterior surface, the polarity of the posterior surface, or a combination thereof.

Dk refers to oxygen permeability, i.e., the amount of oxygen passing through a device such as a dynamic contact lens over a given period of time and pressure difference conditions. Dk is express in units of 10⁻¹¹ (cm/sec)(mL O₂)(mL×mm Hg), also referred to as a barrer. Oxygen transmissibility can be expressed as Dk/t, where t is the thickness of the structure such as a dynamic contact lens and therefore Dk/t represents the amount of oxygen passing through a dynamic contact lens of a specified thickness over a given set of time and pressure difference conditions. Oxygen transmissibility has the units of barrers/cm or 10⁻⁹ (cm/sec)(mL O₂)(mL×mm Hg).

Eye health is promoted by lens materials having oxygen permeability. For dynamic contact lenses, it is generally desirable that the oxygen permeability be greater than about 80 Dk. This high oxygen permeability can be difficult to obtain for high modulus materials and/or for thicker material cross-sections.

The optical portion and the peripheral portion of a dynamic contact lens may comprise a material characterized by an oxygen permeability of at least about 10 Dk, 20 Dk, 30 Dk, 40 Dk, 50 Dk, 60 Dk, 70 Dk, 80 Dk, 90 Dk, 100 Dk, 200 Dk, 300 Dk, 400 Dk, 500 Dk, or more. The optical portion and the peripheral portion of a dynamic contact lens may comprise a material characterized by an oxygen permeability of at most about 500 Dk, 400 Dk, 300 Dk, 200 Dk, 100 Dk, 90 Dk, 80 Dk, 70 Dk, 60 Dk, 50 Dk, 40 Dk, 30 Dk, 20 Dk, 10 Dk, or less. The optical portion and the peripheral portion of a dynamic contact lens may comprise a material characterized by an oxygen permeability that is within a range defined by any two of the preceding values. The optical portion and the peripheral portion of a dynamic contact lens can comprise a material characterized by an oxygen permeability from about 10 Dk to about 500 Dk, from about 50 Dk to about 400 Dk, from about 50 Dk to about 300 DK, and in certain embodiments from about 50 DK to about 100 Dk.

A dynamic contact lens may comprise silicone or silicone hydrogel having a low ionoporosity. For example, a dynamic contact lens may comprise silicone hydrogel or silicone comprising a low ion permeability, and the range of water can be from about 5% to about 35%, such that the Dk is 100×10⁻¹¹ or more. The low ion permeability may comprise an Ionoton Ion Permeability Coefficient of at least about 0.01×10⁻³ cm²/sec, 0.02×10⁻³ cm²/sec, 0.03×10⁻³ cm²/sec, 0.04×10⁻³ cm²/sec, 0.05×10⁻³ cm²/sec, 0.06×10⁻³ cm²/sec, 0.07×10⁻³ cm²/sec, 0.08×10⁻³ cm²/sec, 0.09×10⁻³ cm²/sec, 0.1×10⁻³ cm²/sec, 0.15×10⁻³ cm²/sec, 0.2×10⁻³ cm²/sec, 0.25×10⁻³ cm²/sec, or more. The low ion permeability may comprise an Ionoton Ion Permeability Coefficient of at most about 0.25×10⁻³ cm²/sec, 0.2×10⁻³ cm²/sec, 0.15×10⁻³ cm²/sec, 0.1×10⁻³ cm²/sec, 0.09×10⁻³ cm²/sec, 0.08×10⁻³ cm²/sec, 0.07×10⁻³ cm²/sec, 0.06×10⁻³ cm²/sec, 0.05×10⁻³ cm²/sec, 0.04×10⁻³ cm²/sec, 0.03×10⁻³ cm²/sec, 0.02×10⁻³ cm²/sec, 0.01×10⁻³ cm²/sec, or less. The low ion permeability may comprise an Ionoton Ion Permeability Coefficient that is within a range defined by any two of the preceding values. The low ion permeability may comprise an Ionoton Ion Permeability Coefficient of no more than about 0.25×10⁻³ cm²/sec, for example no more than about 0.08×10⁻³ cm²/sec.

A dynamic contact lens may comprise a wettable surface coating disposed on at least the anterior surface of the dynamic contact lens, such that the tear film is smooth over the dynamic contact lens. The wettable surface coating may comprise a lubricious coating for patient comfort, for example to lubricate the eye when the patient blinks. The wettable coating may create a contact angle no more than about 80°. For example, the coating may create a contact angle no more than about 70°, and the contact angle can be within a range from about 55° to 65° to provide a surface with a smooth tear layer for vision. For example, the wettable coating can be disposed on both an upper surface and a lower surface of the device, i.e., on the anterior and posterior surface of the dynamic contact lens. The upper surface may comprise a wettable coating extending over at least the inner optic portion.

A wettable coating may comprise one or more suitable materials. For example, the wettable coating may comprise polyethylene glycol (PEG), and the PEG coating can be disposed on Parylene™. Alternatively, or in combination, the wettable coating can comprise a plasma coating, and the plasma coating may comprise a luminous chemical vapor deposition (LCVD) film. For example, the plasma coating may comprise at least one of a hydrocarbon, for example, CH₄, O₂, or fluorine containing hydrocarbon, for example, CF₄ coating. Alternatively, or in combination, a wettable coating may comprise a polyethylene glycol (PEG) coating or 2-hydroxyethylmethacrylate (HEMA). For example, a wettable coating may comprise HEMA disposed on a Parylene™ coating, or a wettable coating

A dynamic contact lens provided by the present disclosure can have a water content, for example, from 10 wt % to 70 wt %, such as from 30 wt % to 60 wt %, where wt % is based on the total weight of the dynamic contact lens.

Dynamic contact lenses provided by the present disclosure can be fabricated using any method suitable for fabricating contact lenses and in particular soft contact lenses. Examples of suitable methods include compression molding. The dynamic contact lenses can be fabricated such that as fabricated, the optical portion bulges outward to form a dome, a para-central bulge, or other anteriorly directed surface profile.

Methods of fabricating a dynamic contact lens comprise, for example, forming a dynamic contact lens comprising: an optical portion, wherein the optical portion comprises a sagittal height and a center thickness, wherein the center thickness is less than the sagittal height; and a peripheral portion is coupled to the optical portion, wherein the peripheral portion is configured to retain the dynamic contact lens on the cornea. Methods of fabricating a dynamic contact lens comprise, for example, forming a dynamic contact lens comprising: an optical portion characterized by an optical posterior base curvature; and a peripheral portion is coupled to the optical portion, wherein the peripheral portion comprises a peripheral base curvature, wherein the optical posterior base curvature is different than the peripheral posterior base curvature. For example, the radius of the curvature of the optical portion can be less than the radius of curvature of the peripheral portion. For example, the radius of the curvature of the optical portion can be less than the radius of curvature of the para-central peripheral portion, where the para-central peripheral portion is the part of the peripheral portion adjoining the transition zone and the optical portion. The material used to fabricate a dynamic lens can be a material suitable for use in conventional soft contact lenses. The material can comprise, for example, a Young's modulus from 0.05 MPa to 30 MPa, from 0.1 MPa to 20 MPa, from 0.1 MPa to 10 MPa, from 0.1 MPa to 5 MPa, or from 0.1 MPa to 2 MPa.

Dynamic contact lenses provided by the present disclosure can be fabricated with an as-fabricated SAG height. The as-fabricated center sagittal height refers to the distance from the posterior surface at the center of the optical portion to the extension of the base curve for the paracentral peripheral portion adjacent the optical portion. The as-fabricated center SAG is shown as element 110 in FIG. 1 where the dashed line is the extension of the base curve of the paracentral peripheral portion beneath the optical portion. The as-fabricated SAG height is the maximum gap that can be achieved when the lens is placed on the cornea and the optical portion if filled with tear fluid to form a lenticular tear volume. Depending on a number of factors including the availability of tear fluid, an optical portion with an as-fabricated sagittal height of 40 μm, can produce, for example, a quasi-stable tear volume having a gap of 40 μm, 30 μm, 20 μm, and/or 10 μm. An optical portion with an as-fabricated SAG height of 100 μm, can produce, for example, a quasi-stable tear volume having a gap of 100 μm, 90 μm, 80 μm, 70 μm, 60 μm, 50 μm, 40 μm, 30 μm, and/or 10 μm.

Dynamic contact lenses provided by the present disclosure can be used to correct or to improve vision.

Methods for correcting vision in a patient can comprise applying a dynamic contact lens provided by the present disclosure to an eye of a patient in need of corrected vision.

Correcting vision can comprise correcting hyperopia, myopia, astigmatism, or presbyopia.

Methods provided by the present disclosure comprise treating presbyopia by applying a dynamic contact lens provided by the present disclosure to a presbyopic eye of a patient.

Dynamic contact lenses provided by the present disclosure can be designed to dynamically correct vision. For example, presbyopia is characterized by the inability of the eye to focus on close objects. The optical portion of dynamic contact lenses provided by the present disclosure can dynamically change configuration to accommodate either distant vision or near vision. For example, as relevant to presbyopia, in a first configuration appropriate for viewing distant objects the optical portion of a dynamic contact lens can lie proximate the cornea. In this configuration there is no substantial tear volume and distance vision is uncorrected. Then, when the patient views a near object, the optical portion of the dynamic contact lens can assume a second configuration that corrects the presbyopia and facilitates clearly viewing near objects. This is accomplished without changing the radial thickness of the optical portion or by changing the ratio of the optical posterior curvature to the optical anterior curvature of the optical portion. Rather, as the optical portion bulges outward, the lenticular optical tear volume expands to provide a tear volume that serves to dynamically correct the near-term vision by changing the curvature of the anterior surface of the optical portion.

Dynamic contact lenses provided by the present disclosure can also be used as multi-focal lenses to correct presbyopia and to prevent progression of myopia.

Static configurations of dynamic contact lenses provided by the present disclosure can be used to compensate for an irregular cornea, to treat astigmatism, or for corneal wound healing.

Dynamic contact lenses incorporating a tear volume can correct vision resulting from an irregularly shaped cornea. An irregularly shaped cornea can be permanent or temporary such as resulting from ocular surgery including photorefractive keratectomy or corneal cross-linking procedures. The tear volume can correct astigmatism. For treatment of such conditions a dynamic contact lens provided by the present disclosure having a static tear volume can be appropriate.

Dynamic contact lenses provided by the present disclosure can be used to enhance or to restore visual acuity following ocular therapy. Ocular therapies can involve manipulation of the ocular tissue and can be associated with a lesion external to the optical region. Ocular therapies can involve incising an ocular tissue and implanting a device within the optical region. In certain embodiments, ocular therapies involve ablating at least a portion of the stroma and/or epithelium. Ocular therapies can include, for example, cataract surgery including phacoemulsification, conventional extracapsular cataract extraction, and intracapsular cataract extraction; glaucoma surgery including laser trabeculoplasty, irdotomy, irdectomy, sclerotomy, goniotomy, drainage implant surgery, and canaloplasty; corneal surgery including corneal transplant surgery, penetrating keratopalsty, keratoprosthesis, pterygium excision, corneal tattooing, and osteo-ondonto-keratoprosthesis; and photorefractive therapy including photorefractive keratectomy (PRK) and laser-assisted in-situ keratomileusis (LASIK). Ocular therapy can also involve treating a wound to the eye, wherein the treatment may or may not involve ocular surgery. Ocular therapy can comprise cataract surgery, corneal inlay surgery, corneal transplant surgery, or treatment of an ocular trauma wound. Ocular therapy can comprise incising the cornea and/or perforating the cornea at a site external to the optical region.

In general, ocular therapies such as cataract surgery, corneal inlay surgery, and corneal transplant surgery can be distinguished from ocular therapies involving manipulation only to the optical region of the cornea or primarily to the optical region of the cornea. In the former ocular therapies, which can be considered implantation surgeries in that a device is implanted into an ocular tissue as an adjunct or as a replacement for an ocular tissue that is removed, the procedures involve manipulation of ocular tissue external to the optical region as well as to the optical region itself. The latter therapies are exemplified by refractive surgeries in which the optical region of the cornea is sculpted to correct refractive visual error. Examples of refractive surgeries include, for example, PRK and LASIK. Ocular therapies involving manipulation of the optical region of the cornea are encompassed to the extent that the therapy also involves manipulation of ocular tissue external to the optical region. For example, LASIK involves making an incision in the stroma external to the optical region to form a flap. The flap is then lifted back to expose the stroma, which is then ablated using a laser to provide a shape for refractive correction. Furthermore, ocular manipulation involving tissue external to the optical region and photorefractive surgery involving manipulation of tissue within the optical region can be combined. For example, corneal inlay surgery and associated photorefractive surgery such as LASIK surgery can be combined.

Dynamic contact lenses provided by the present disclosure may be used to treat the cornea following corneal inlay surgery or corneal onlay surgery. Corneal inlays and onlays are small lenses or other optical devices inserted into the cornea to reshape the front surface of the eye, i.e., the anterior surface of the cornea, to improve vision and in some cases, can resemble small contact lenses. The primary use of current corneal inlays is to improve near vision and to address presbyopia. In some cases, corneal inlay surgery can be combined with photorefractive surgery such as LASIK to correct both presbyopia and common refractive errors such as nearsightedness, farsightedness, and/or astigmatism.

Dynamic contact lenses provided by the present disclosure may be used to treat the cornea following cataract surgery. In certain embodiments, ocular therapy comprises cataract surgery. Cataract surgery involves the removal and replacement of the natural lens of the eye that has developed opacification, which is referred to as a cataract.

Dynamic contact lenses provided by the present disclosure may be used to treat the cornea following corneal transplantation surgery. Corneal transplantation therapies include, for example, penetrating keratoplasty, lamellar keratoplasty, deep anterior lamellar keratoplasty, and endothelial keratoplasty.

Dynamic contact lenses provided by the present disclosure when applied to an eye of a patient following ocular therapy speed healing of ocular defects. Ocular defects include incisions and perforations of the cornea and/or other ocular tissue.

Dynamic contact lenses provided by the present disclosure may be used to treat the cornea following cross-linking therapy. Corneal cross-linking is a technique that strengths the chemical bonds in the cornea and thereby facilitate the ability of the cornea to resist irregular changes to the corneal shape known as ectasia.

Dynamic contact lenses provided by the present disclosure may be used to treat the cornea following photorefractive therapy such as, for example, PRK and LASIK. Refractive eye surgery is used to improve the refractive state of the eye and includes procedures such as, for example, automated lamellar keratoplasty (ALK), laser assisted in-situ keratomileusis (LASIK), photorefractive keratectomy (PRK), laser assisted sub-epithelium keratomileusis (LASEK), EPI-LASIK, radial keratotomy, mini-asymmetric radial keratotomy, arcuate keratotomy, limbal relaxing incisions, thermal keratoplasty, laser thermal keratoplasty, intrastromal corneal ring segment removal, and phakic intraocular lens implantation. Following any of these procedures there is a period of time before optimal vision is restored. For example, in LASIK, optimal vision is typically achieved within about 24 hours following surgery. During this recovery period, in addition to sub-optimal visual acuity, a patient may experience discomforts such as photophobia or light sensitivity and/or a burning sensation. Methods for reducing the time to achieve optimal vision and for reducing or eliminating discomfort associated with refractive eye surgery are desired.

PRK is a surgical procedure in which a laser is used to shape the stroma to correct for photorefractive error. In the process, the epithelium overlying the portion of the ablated stroma is removed to form an epithelial defect.

LASIK is a surgical procedure used to correct refractive vision errors such a myopia, hyperopia, and astigmatism in which a laser is used to reshape the cornea to improve visual acuity, e.g., the clearness and sharpness of an image. The LASIK procedure involves both a surgical cutting and laser sculpting of the cornea. During LASIK, the eye is immobilized by application of a soft corneal suction ring. A flap in the outer cornea is then created using a blade or laser leaving a hinge on one end of the flap. The flap is then folded back to expose the stroma, or middle section of the cornea. A laser is then used to vaporize the corneal stroma to remove tissue to reshape the cornea to correct vision. After the stromal layer is reshaped, the flap is repositioned over the eye and remains in position by natural adhesion. Optimal visual acuity is usually achieved within about 24 hours following surgery.

Dynamic contact lenses provided by the present disclosure can be configured to correct refractive error such as astigmatism. The lenses provide a smooth spherical anterior surface and minimize lens-induced distortions by reducing flexure of the inner optical portion and by maintaining lens centration during wear. Reduced flexure of the inner optical portion can, in part, be accomplished by increasing the rigidity of the inner portion and by creating a tear volume. Centration of the inner optical portion minimizes astigmatic and prismatic effects caused by tilting of the optic and also minimizes edge distortion.

While the foregoing has focused on ocular therapies associated with intentional manipulation of the eye, it can be appreciated that dynamic contact lenses and methods of using the dynamic contact lens can also be useful in treating other injuries to the eyes such as, for example, the treatment of trauma wounds. Trauma to the eye can also cause edema and compromise the interfaces between the various ocular tissue. Thus, in addition to post-surgical methods, dynamic contact lenses provided by the present disclosure are useful in healing trauma wounds to the eye. Trauma includes, for example, physical trauma such as blunt trauma and penetrating trauma, chemical trauma, blast injury, burn, and psychological trauma. Treatment of a trauma wound may involve surgical procedures such as removing an embedded physical object or removing scar tissue. To the extent that the trauma produces edema and optical irregularities, application of a dynamic contact lens will lead to faster visual recovery and, by stabilizing the involved ocular tissue, accelerate healing. Trauma may also cause defects to ocular tissue including to the anterior surface of the cornea and involve the epithelium and/or stroma and may cause damage to internal ocular tissue. Wound healing thus includes healing wounds associated with physical damage to ocular tissue not necessarily caused by surgical procedures.

Dynamic contact lenses provided by the present disclosure may also be used as preventative devices. For example, dynamic contact lenses may be used to protect an eye from a potential injury such as injury due to physical trauma, protection from chemicals, protection from particulates, and protection from edema. As a preventative device, a dynamic contact lens can be applied to the eye prior to an anticipated exposure to a potential injury. When worn for protecting the eye from a potential injury, a dynamic contact lens can provide a physical barrier, a chemical barrier by virtue of the seal to the anterior surface of the eye, and/or may prevent or minimize edema caused by non-physical force such as blast pressure or by trauma to other parts of the body. In certain embodiments, protecting the eye form potential injury includes protected the eye from gases, vapors, dust, or smoke. In certain embodiments, protecting includes protected from edema.

### EXAMPLES

Embodiments provided by the present disclosure are further illustrated by reference to the following examples, which describe dynamic contact lenses and uses of dynamic contact lenses provided by the present disclosure.

### Example 1: Optical Function of a Dynamic Contact Lens in an Eye Model

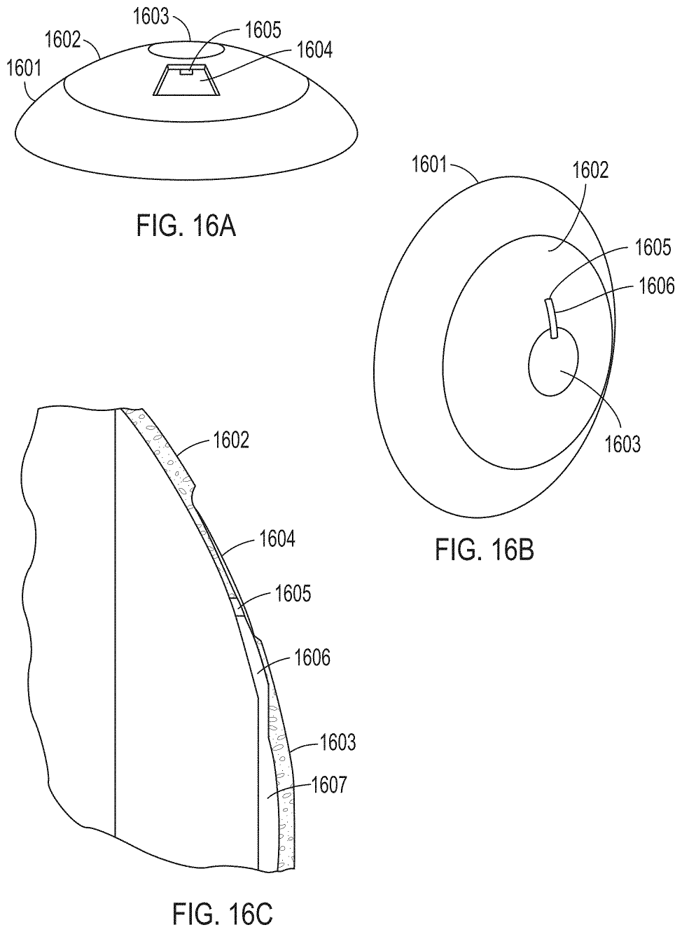

OCT images of a dynamic contact lens having a transitioning mechanism is shown in FIGS. 20A and 20B. The dynamic contact lens 2002 overlies a cornea 2001. As shown in FIG. 20A, a fenestration 2005 couples tear fluid of the tear meniscus 2006 with a groove 2004 disposed on the posterior surface of the peripheral portion 2007 of the dynamic contact lens 2002. The groove 2004 is chamfered toward the optical portion 2008 of the dynamic contact lens such that the groove 2004 fluidly couples tear fluid from the tear meniscus to the tear volume 2003 formed between the posterior surface of the optical portion and the anterior surface of the cornea.

A close-up view of the optical tear volume 2003 and the fluid coupling with groove 2004 is shown in FIG. 20B.

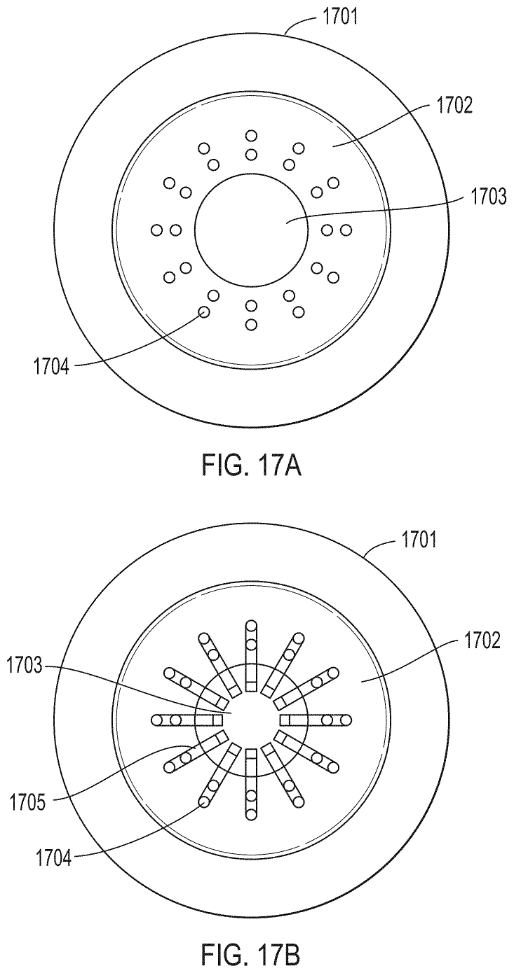

FIGS. 21A and 21B show horizontal and vertical OCT images of a dynamic contact lens 2102 on the cornea 2101 of the patient to further illustrate the coupling of the tear fluid at the tear meniscus 2106 with the groove 2104 via the fenestration 2105.

FIG. 21C shows an OCT image of a dynamic contact lens on a cornea showing fluid coupling of a tear meniscus to a groove with a gap height of 68 μm between the posterior surface of the optical portion and the anterior surface of the cornea. The groove has dimensions of 498 μm.

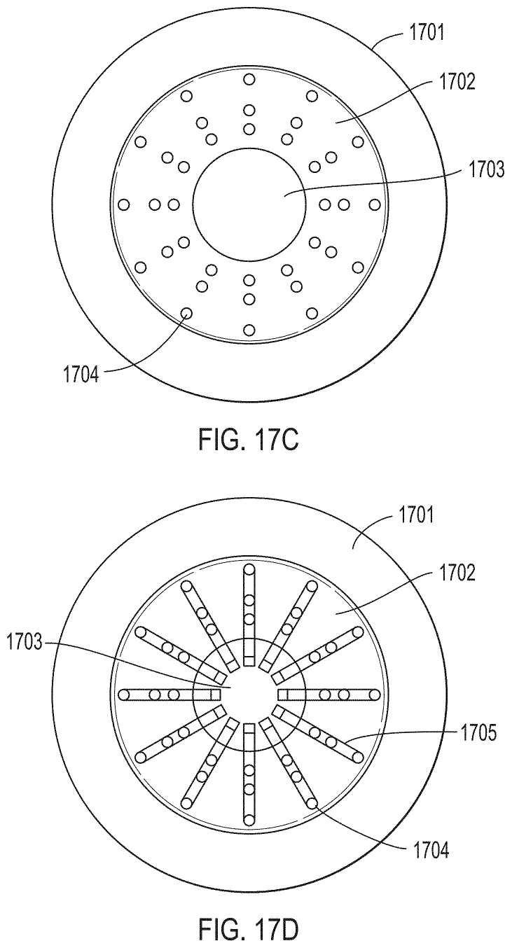

FIG. 22 shows an OCT image of the tear volume 2203 formed between the optical portion of the dynamic contact lens 2202 and the cornea 2201 during downward gaze of the eye. During downward gaze the eyelids exert pressure on the peripheral portion of the dynamic contact lens and push tear fluid into the volume between the optical portion and the cornea to change the tear volume. Alternatively, or in addition to, fluid coupling of the optical tear volume to a source of tear fluid such as a tear meniscus during downward gaze can change the lens forces such as to cause the optical tear volume to bulge away from the cornea. As shown in FIG. 22, in this example, optical tear volume 2203 has a maximum gap height of 29 μm.

### Example 2: Dynamic Contact Lens with Fenestrations and Grooves

Dynamic contact lenses were fabricated having the parameters shown in Table 2.

TABLE 2
Parameters of the dynamic contact lens of Example 2.

| Lens Element | Parameter | Value | |
|---|---|---|---|
| Dynamic contact lens | EBC (average base curvature) | 8.9 | mm |
| | Material | silicone hydrogel | |
| Optical Portion | As-fabricated SAG | 0.1 | mm |
| | Center thickness | 200 | μm |
| | Base curvature | 7.86 | mm |
| | Diameter | 4 | mm |
| | tear volume (calculated) | 0.0114 | μL |
| Peripheral Portion | Maximum thickness | 200 | μm |
| | Base curvature | 9.32 | mm |
| | Diameter | 10 | |
| Transition Zone | Abrupt | Abrupt | |
| Grooves | Number | 12 | |
| | Disposition | 30 | degrees |
| | Width | 0.4 | |
| | Depth | 140 | μm |
| | Length | 3.5 | mm |
| | Radial position | 2.5 | mm |
| Fenestrations | Number | 12 | |
| | Diameter | 0.4 | mm |
| | Radial location | 3.5 | mm |

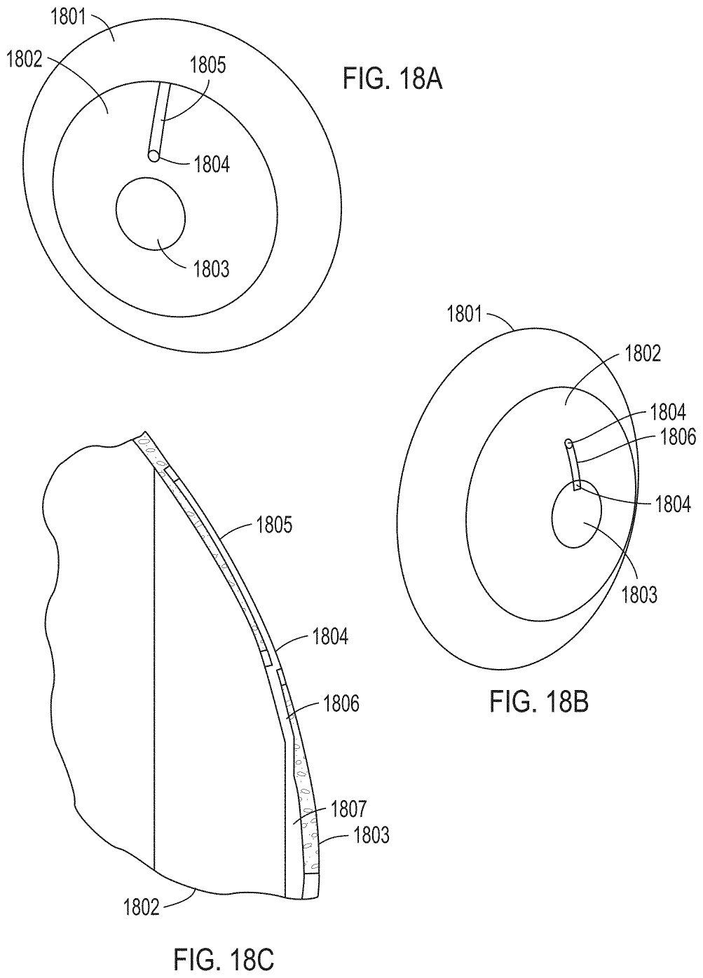

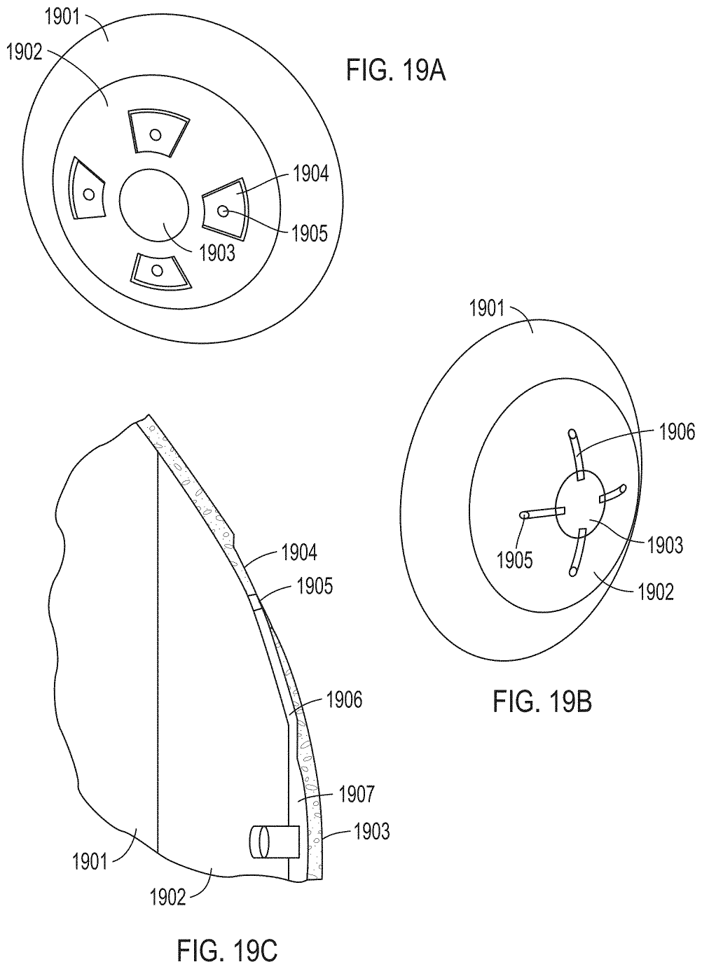

The dynamic contact lens was placed onto a cornea of a patient. FIG. 23 shows a photograph of the dynamic contact lens on an eye of a patient and fenestrations 2301 are evident on the left side of the eye outside the optical region of the eye. An OCT image of the dynamic contact lens on the cornea in forward gaze is shown in FIG. 24. FIG. 24 shows the dynamic contact lens 2401, cornea 2402, optical portion 2403, fenestration 2404, and groove 2405, which tapers toward the optical portion 2403. Although difficult to visualize from the OCT image, the gap height was about 10 μm to 15 μm.

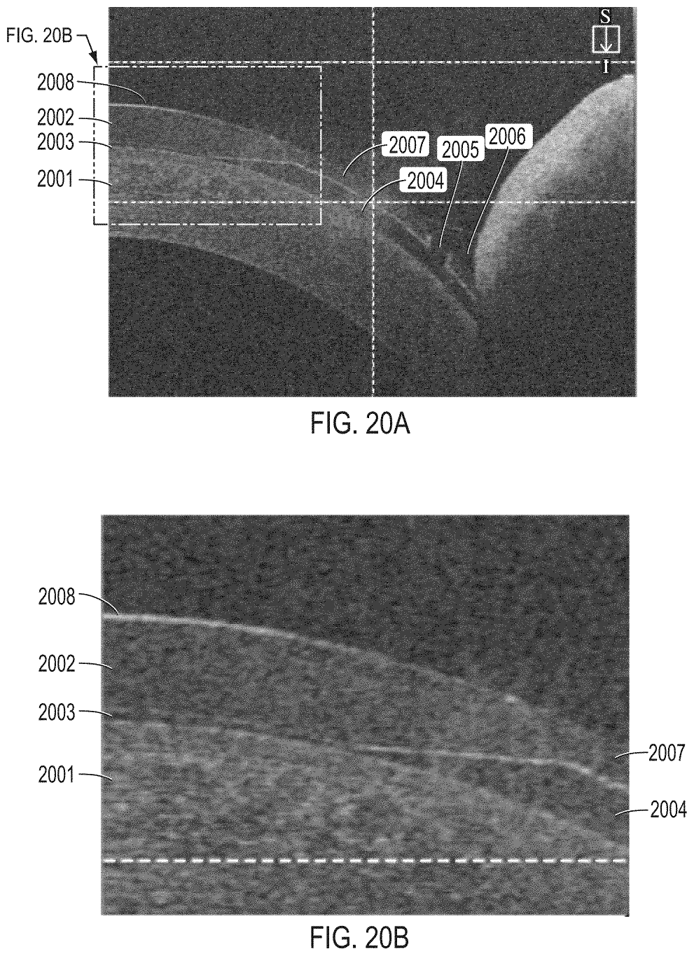

Using a micropipette adapted for low volume, 0.1 μL of tear fluid was placed over the lower fenestration. An OCT image of the dynamic contact lens and cornea with the patient gazing straight ahead is shown in FIG. 25. As shown in FIG. 25, the optical portion 2503 immediately bulged anteriorly to provide a 70 μm-gap between the center of the optical portion 2503 and the cornea 2502. FIG. 25 shows an OCT image of dynamic contact lens including optical portion 2501, cornea 2502, tear volume 2503, fenestration 2504, and posterior groove 2505 extending into the transition zone 2506.

### Example 3: Dynamic Contact Lens with Fenestrations and Grooves

A dynamic contact lens was fabricated having the parameters shown in Table 3.

TABLE 3
Parameters of the dynamic contact lens of Example 3.

| Lens Element | Parameter | Value | |
|---|---|---|---|
| Dynamic contact lens | EBC | 8.9 | |
| | Material | silicone hydrogel | |
| Optical Portion | As-fabricated SAG | 0.1 | mm |
| | Center thickness | 200 | μm |
| | Base curvature | 7.86 | mm |
| | Diameter | 3 | mm |
| Peripheral Portion | Maximum thickness | 200 | μm |
| | Base curvature | 9.32 | mm |
| | Diameter | 10 | mm |
| Transition Zone | Abrupt | Abrupt | |
| Grooves | Number | 1 | |
| | Disposition | posterior surface | |
| | Width | 400 | μm |
| | Depth | 100 | μm |
| | Length | 2.5 | mm |
| | Radial position | 2.5 | mm |
| Fenestrations | Number | 1 | |
| | Diameter | 400 | μm |
| | Radial location | 2.5 | mm |

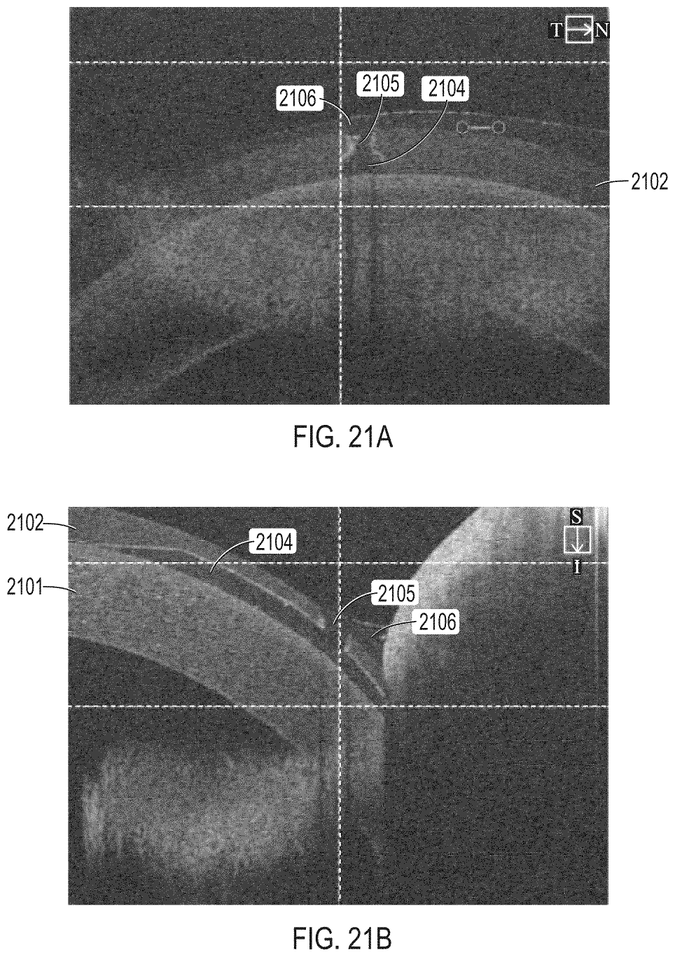

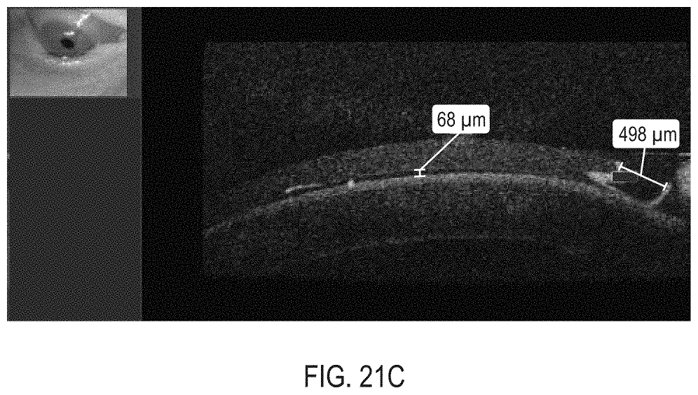

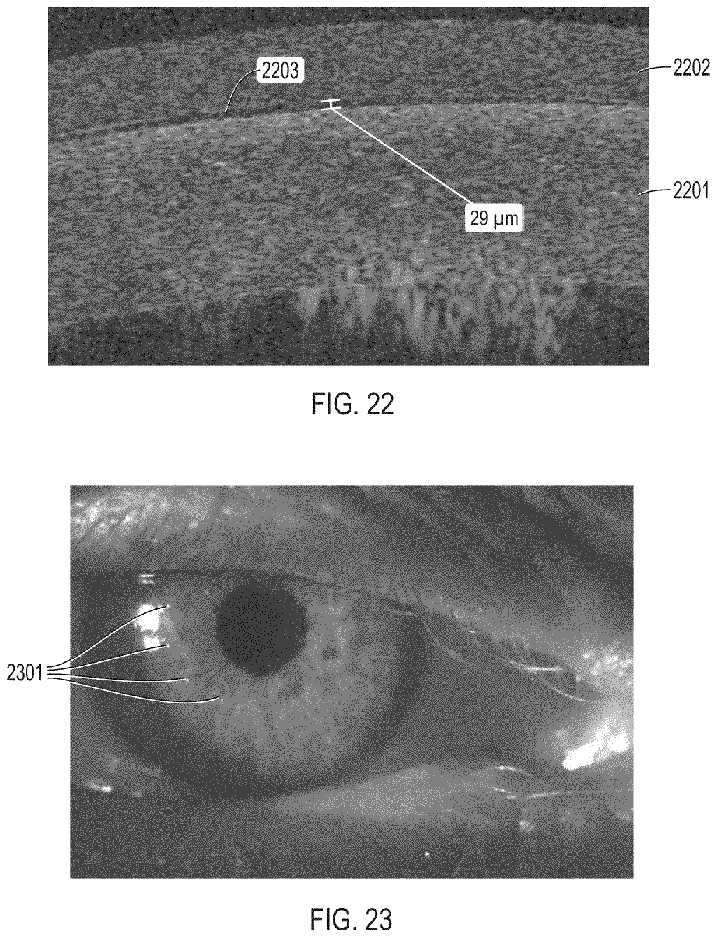

The dynamic contact lens characterized by the parameters in Table 2 was placed on the eye of the patient. On primary gaze (forward gaze) the optical portion of the lens was flattened in a conforming configuration against the cornea. A shown in FIG. 26 (and shown on the upper horizontal OCT section) the posterior groove was 1 mm below (FIG. 27) and the fenestration was 2.5 mm below the center (FIG. 28) and about 2.5 mm above the tear meniscus. FIG. 26 shows an OCT image of the dynamic contact lens overlying the cornea 2602 with the optical portion 2601 substantially conforming to cornea 2602 with a small tear volume 2603. FIG. 27 shows an OCT image of a cross-section of the dynamic contact lens showing posterior groove 2707. FIG. 28 shows an OCT image of a cross-section of the dynamic contact lens 2801 overlying cornea 2802 and showing posterior groove 2807 and fenestration 2808.

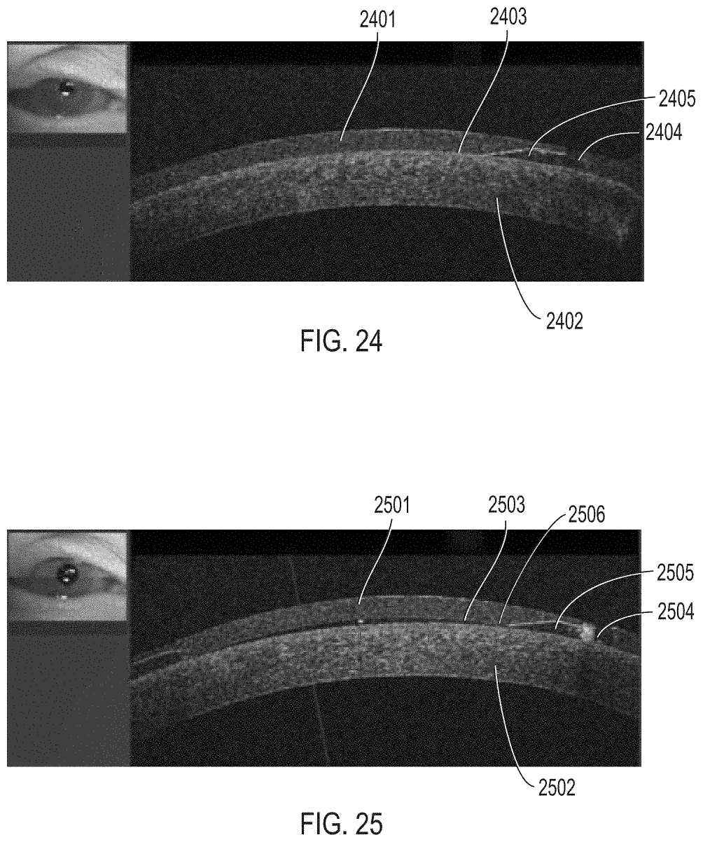

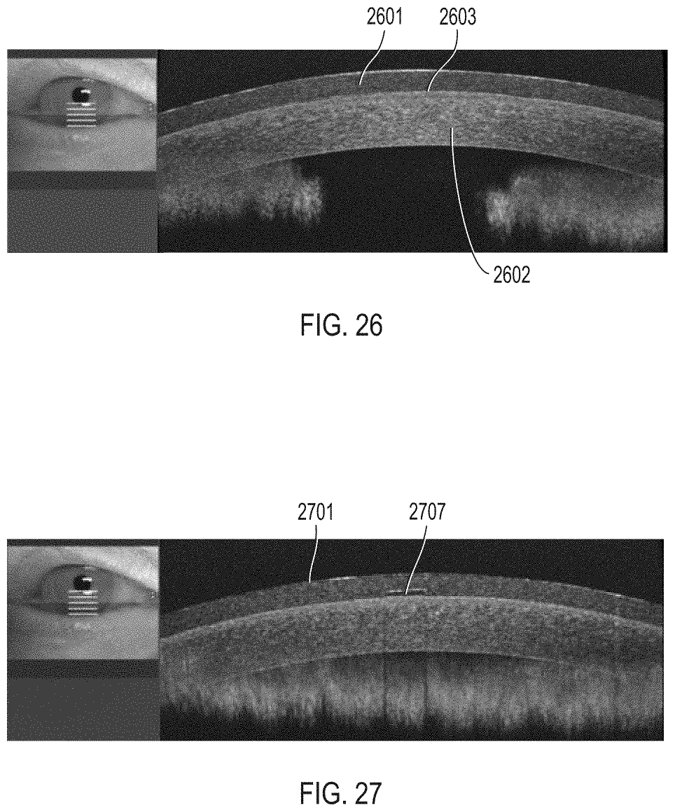

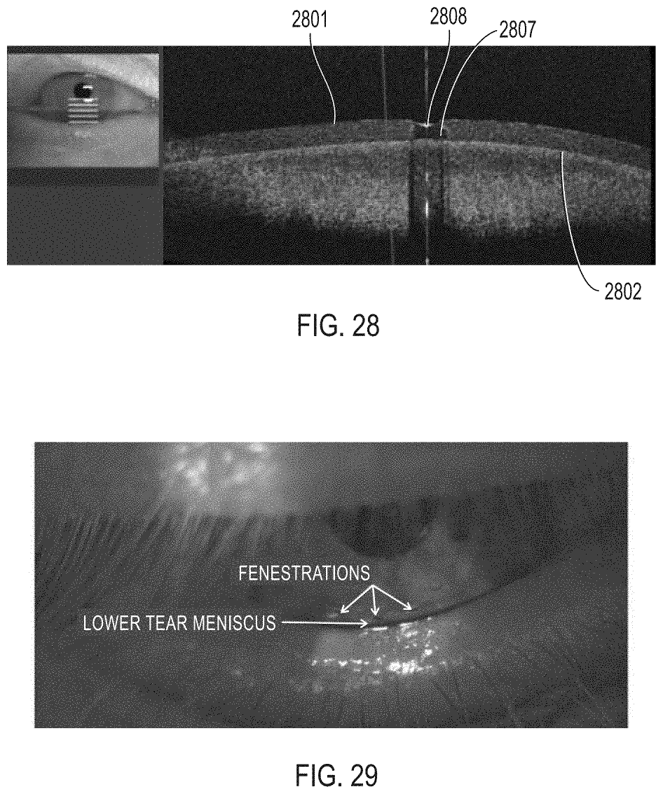

The patient then diverted his gaze downward to about 40 degrees in a reading position. As shown in the FIG. 29, during the downward gaze, the lower fenestrations became fluidly coupled to the tear meniscus, thereby fluidly coupling the tear meniscus to the optical tear volume through the fenestration and the groove. When the fenestration was fluidly coupled to the tear meniscus, the optical portion immediately bulged anteriorly as shown in FIG. 30 and a gap of 40 μm forming between the posterior surface of the lens and the cornea. FIG. 30 shows an OCT image of dynamic contact lens 3001 overlying cornea 3002, and with a tear volume 3006 between the posterior surface of optical portion 3003 and cornea 3002. FIG. 31 is an OCT image showing a posterior groove 3107 during downward gaze.

### Further Aspects of the Invention

Aspect 1. A contact lens comprising: an optical portion, wherein the optical portion comprises an optical posterior base curvature; and an optical center; a peripheral portion, wherein the peripheral portion comprises a peripheral posterior base curvature; and a transition zone coupling the optical portion and the peripheral portion, wherein, the transition zone is located at a radius less than 3.5 mm from the optical center; the central base curvature (also referred to herein as the optical posterior base curvatures) is less than 7.4 mm; and the peripheral base curvature is at least 0.4 mm greater than the center base curvature.

Aspect 1.1. The contact lens of aspect 1, wherein the optical posterior base curvature is less than 7.3 mm.

Aspect 1.2. The contact lens of aspect 1, wherein the optical posterior base curvature is less than 7.2 mm.

Aspect 1.3. The contact lens of aspect 1, wherein the optical posterior base curvature is less than 7.1 mm.

Aspect 1.4. The contact lens of aspect 1, wherein the optical posterior base curvature is less than 7.0 mm.

Aspect 1.5. The contact lens of aspect 1, wherein the optical posterior base curvature is less than 6.9 mm.

Aspect 1.6. The contact lens of aspect 1, wherein the optical posterior base curvature is less than 6.8 mm.

Aspect 1.7. The contact lens of aspect 1, wherein the optical posterior base curvature is less than 6.7 mm.

Aspect 1.8. The contact lens of aspect 1, wherein the optical posterior base curvature is less than 6.6 mm.

Aspect 1.9. The contact lens of aspect 1, wherein the optical posterior base curvature is less than 6.5 mm.

Aspect 1.10. The contact lens of aspect 1, wherein a curvature immediately adjacent to the central base curvature is at least 0.2 mm greater than the central base curvature.

Aspect 2. A contact lens comprising: an optical portion wherein the optical portion comprises an optical posterior base curvature; a peripheral portion wherein the peripheral portion comprises a peripheral posterior base curvature; and a transition zone coupling the optical portion and the peripheral portion, wherein, the transition zone comprises a radial width of 150 microns or less

Aspect 3. A contact lens comprising: an optical portion, wherein the optical portion comprises an optical posterior base curvature; a peripheral portion, wherein the peripheral portion comprises a peripheral posterior base curvature; and a transition zone coupling the optical portion and the peripheral portion, wherein the transition zone comprises a circumference and a thickness, wherein the thickness varies around the circumference of the transition zone.

Aspect 4. A contact lens comprising: an optical portion, wherein the optical portion comprises an optical center, an optical posterior base curvature, and an optical posterior surface; a peripheral portion, wherein the peripheral portion comprises a peripheral posterior base curvature, a peripheral posterior surface, and a peripheral anterior surface; a transition zone coupling the optical portion and the peripheral portion; one or more grooves in the peripheral posterior surface, wherein at least one groove extends from the peripheral posterior surface to the optical portion; and at least one fenestration connecting the at least one groove to the peripheral anterior surface, wherein, the transition zone comprises a circumference and a thickness; the thickness varies around the circumference of the transition zone; the optical posterior base curvature is less than 7.1 mm; and the peripheral posterior base curvature is at least 0.4 mm greater than the optical posterior base curvature at a radius less than 3.5 mm from the optical center.

Aspect 5. A contact lens comprising an optical portion, wherein the optical portion comprises an optical center, an optical posterior base curvature, and an optical posterior surface; and a peripheral portion coupled to the optical portion, wherein the peripheral portion comprises a peripheral posterior base curvature, a peripheral diameter, a peripheral posterior surface, and a peripheral anterior surface; wherein the contact lens is configured such that when worn on an eye of a patient, the optical portion forms a lenticular volume between the cornea and the optical posterior surface; and wherein the lenticular volume comprises a diameter of at least 1.5 mm and a height of at least 0.01 mm over the cornea.

Aspect 6. A contact lens comprising: an optical portion, wherein the optical portion comprises an optical posterior base curvature; and a peripheral portion coupled to the optical portion, wherein the peripheral portion comprises a peripheral posterior base curvature, and a peripheral diameter; wherein the contact lens is configured such that, when worn on an eye of a patient, the optical portion can assume a first quasi-stable configuration and a second quasi-stable configuration.

Aspect 7. A contact lens comprising: An optical portion, wherein the optical portion comprises an optical posterior surface; A peripheral portion, wherein the peripheral portion comprises a peripheral posterior surface; and a transition zone coupling the optical portion and the peripheral portion, wherein the contact lens is configured such that when worn on the eye of a patient, the optical portion can assume a plurality of configurations in response to a pressure applied to the optical portion; wherein when a negative pressure is applied to the optical posterior surface, the optical posterior surface assumes one or more substantially conforming configurations with respect to the anterior surface of the cornea; and wherein in the absence of a negative pressure, the optical posterior surface assumes a neutral configuration to provide a tear volume between the optical posterior surface and the anterior surface of the cornea.

Aspect 8. The contact lens of aspect 7, wherein the in the one or more substantially conforming configurations the thickness of a tear film between the optical posterior surface and the anterior surface of the cornea varies by less than 10 μm.

Aspect 9. The contact lens of aspect 7, wherein the in the one or more substantially conforming configurations the thickness of a tear film between the optical posterior surface and the anterior surface of the cornea varies by less than 3 μm.

Aspect 10. The contact lens of any one of aspects 7 to 9, wherein the negative pressure is from 5 Pa to 1,500 Pa.

Aspect 11. The contact lens of any one of aspects 7 to 9, wherein the negative pressure is from 10 Pa to 250 Pa.

Aspect 12. The contact lens of any one of aspects 2, 3, and 5-11, wherein, the peripheral posterior base curvature is from 7.5 mm to 9.5 mm; and the difference between the peripheral posterior base curvature and the optical posterior base curvature is greater than 0.4 mm.

Aspect 13. The contact lens of any one of aspects 1 to 12, wherein the optical posterior base curvature is less than 6.8 mm.

Aspect 14. The contact lens of any one of aspects 1 to 13, wherein the transition zone has a thickness that varies around the circumference of the transition zone.

Aspect 15. The contact lens of any one of aspects 1 to 13, wherein the transition zone has a thickness that varies in a regular pattern around the circumference of the transition zone.

Aspect 16. The contact lens of any one of aspects 1 to 13, wherein the transition zone comprises one or more discontinuities extending across the transition zone.

Aspect 17. The contact lens of aspect 16, wherein the one or more discontinuities comprises one or more posterior grooves in the posterior surface of the peripheral portion and extending into the optical portion.

Aspect 18. The contact lens of aspect 17, wherein of the one or more posterior grooves are coupled to a fenestration.

Aspect 19. The contact lens of aspect 17, wherein of the one or more posterior grooves are coupled to a tear fluid reservoir.

Aspect 20. The contact lens of any one of aspects 1 to 19, wherein, the optical posterior base curvature is less than 7.1 mm; and the peripheral base curvature is at least 0.4 mm greater than the optical posterior base curvature.

Aspect 21. The contact lens of any one of aspects 1 to 20, wherein each of the optical portion and the peripheral portion comprises a material having a modulus from 0.1 MPa to 10 MPa.

Aspect 22. The contact lens of any one of aspects 1 to 21, comprising one or more posterior grooves in the peripheral posterior surface, wherein at least one posterior groove extends from the peripheral posterior surface into the optical portion.

Aspect 23. The contact lens of any one of aspects 4 and 22, wherein each of the one or more grooves extends radially from the center of the optical portion.

Aspect 24. The contact lens of any one of aspects 1 to 23, wherein, when worn on the eye of a patient, the optical portion is characterized by a first quasi-stable configuration and a second quasi-stable configuration, wherein interaction of the contact lens with eye movement causes a transition between the first quasi-stable configuration and the second quasi-stable configuration.

Aspect 25. The contact lens of aspect 24, wherein, the first quasi-stable configuration comprises a first gap height; the second quasi-stable configuration comprises a second gap height; the first gap height and the second gap height are different; and wherein the gap height is the distance between a center of the optical posterior surface and the cornea.

Aspect 26. The contact lens of any one of aspects 24 to 25, wherein eye movement comprises changing a gaze position of the eye.

Aspect 27. The contact lens of any one of aspects 24 to 26, wherein, in the first quasi-stable configuration the optical portion comprises a first optical power; and in the second quasi-stable configuration the optical portion comprises a second optical power, wherein the first optical power is different than the second optical power.

Aspect 28. The contact lens of any one of aspects 24 to 27, wherein, when worn on the eye of a patient, an optical tear volume is formed between the optical posterior surface and the anterior surface of the cornea; in the first quasi-stable configuration the optical tear volume comprises a first volume; and in the second quasi-stable configuration the optical tear volume comprises a second volume; wherein the first volume is different than the second volume.

Aspect 29. The contact lens of any one of aspects 24 to 28, wherein, when worn on the eye of a patient, an optical tear volume is formed between the optical posterior surface and the anterior surface of the cornea; in the first quasi-stable configuration the optical tear volume comprises a first shape; in the second quasi-stable configuration the optical tear volume comprises a second shape; and the first shape is different than the second shape.

Aspect 30. The contact lens of any one of aspects 24 to 29, wherein, the first quasi-stable configuration provides an optical power that focuses an image on the fovea from a first distance; and the second quasi-stable configuration provides an optical power that focuses an image on the fovea from a second distance.

Aspect 31. The contact lens of any one of aspects 1 to 30, wherein, when worn on the eye of a patient, the optical portion is characterized by a first quasi-stable configuration and a second quasi-stable configuration and an optical tear volume is formed between the optical posterior surface and the anterior surface of the cornea; and a transition between the first quasi-stable configuration and the second quasi-stable configuration is controlled by the flow of tear fluid into and out of the optical tear volume.

Aspect 32. The contact lens of any one of aspects 1 to 30, wherein, when worn on the eye of a patient, the optical portion is characterized by a first quasi-stable configuration and a second quasi-stable configuration and an optical tear volume is formed between the optical posterior surface and the anterior surface of the cornea; and a transition between the first quasi-stable configuration and the second quasi-stable configuration is controlled by fluidly coupling and decoupling the optical tear volume with a tear meniscus.

Aspect 33. The contact lens of any one of aspects 1 to 30, wherein, when worn on the eye of a patient, the optical portion is characterized by a first quasi-stable configuration and a second quasi-stable configuration; the contact lens comprises one or more fenestrations connecting the peripheral posterior surface to the anterior posterior surface; and fluidly coupling one or more fenestrations to a tear meniscus causes a change in the optical power of the optical portion.

Aspect 34. The contact lens of any one of aspects 1 to 30, wherein, when worn on the eye of a patient, the optical portion is characterized by a first quasi-stable configuration and a second quasi-stable configuration; the contact lens comprises one or more fenestrations connecting the peripheral posterior surface to the peripheral anterior surface; and fluidly decoupling one or more fenestrations with a tear meniscus causes a change in the optical power of the optical portion.

Aspect 35. The contact lens of any one of aspects 1 to 34, wherein the contact lens comprises: at least one groove in the peripheral posterior surface, wherein the at least one groove extends from the peripheral posterior surface to the optical portion; and at least one fenestration connecting the at least one groove to the peripheral anterior surface.

Aspect 36. The contact lens of any one of aspects 1 to 35, wherein, when worn on an eye of a patient, an optical tear volume is formed between the posterior surface of the optical portion and the anterior surface of the cornea.

Aspect 37. The contact lens of any one of aspects 1 to 36, wherein, when worn on an eye of a patient, a gap is formed between the posterior surface of the optical portion and the anterior surface of the cornea, wherein the gap has a maximum height from 1 μm to 200 μm.

Aspect 38. The contact lens of any one of aspects 1 to 37, wherein the optical portion is centered on the central axis of the contact lens.

Aspect 39. The contact lens of any one of aspects 1 to 37, wherein the optical portion is not centered on the central axis of the contact lens.

Aspect 40. The contact lens of any one of aspects 1 to 37, wherein the optical portion is centered on an axis that is less than 45 degrees from the central axis of the contact lens.

Aspect 41. The contact lens of any one of aspects 1 to 40, wherein the optical portion comprises a maximum thickness within a range from 30 μm to 600 μm.

Aspect 42. The contact lens of any one of aspects 1 to 41, wherein the optical portion comprises a maximum rigidity within a range from 2E3 MPa×μm³ to 3E9 MPa×μm³.

Aspect 43. The contact lens of any one of aspects 1 to 42, wherein the optical portion, the peripheral portion, or both the optical portion and the peripheral portion comprise at least one mechanism configured to transport tear fluid into and out of an optical tear volume formed between the optical posterior surface and the anterior surface of the cornea, when worn on an eye of a patient.

Aspect 44. The contact lens of aspect 43, wherein the transport of tear fluid into and out of the optical tear volume is associated with a transition between a first quasi-stable configuration of the optical portion and a second quasi-stable configuration of the optical portion.

Aspect 45. The contact lens of aspect 43, wherein the at least one mechanism comprises a posterior groove, an anterior groove, a fenestration, a tear fluid reservoir, a protrusion, a depression, a valve, a fenestration comprising a valve, a geometry of the optical portion, a geometry of the peripheral portion, or a combination of any of the foregoing.

Aspect 46. The contact lens of any one of aspects 43 to 45, wherein interaction of tear fluid in the tear meniscus with the optical tear volume induces a transition between a first quasi-stable configuration of the optical portion and a second quasi-stable configuration of the optical portion, maintains a first quasi-stable configuration of the optical portion, maintains a second quasi-stable configuration of the optical portion, or a combination of any of the foregoing.

Aspect 47. The contact lens of any one of aspects 43 to 46, wherein motion of the eye, an eyelid, or a combination thereof, induces a transition between a first quasi-stable configuration of the optical portion and a second quasi-stable configuration of the optical portion, maintains a first quasi-stable configuration of the optical portion, maintains a second quasi-stable configuration of the optical portion, or a combination of any of the foregoing.

Aspect 48. The contact lens of any one of aspects 43 to 47, wherein interaction of tear fluid in the tear meniscus with at least two of the optical portion, the peripheral portion, and the at least one mechanism, induces a transition between a first quasi-stable configuration of the optical portion and a second quasi-stable configuration of the optical portion, maintains a first quasi-stable configuration of the optical portion, maintains a second quasi-stable configuration of the optical portion, or a combination of any of the foregoing

Aspect 49. The contact lens of any one of aspects 43 to 47, wherein interaction between tear fluid within the optical tear volume and tear fluid within a tear fluid source induces a transition between a first quasi-stable configuration of the optical portion and a second quasi-stable configuration of the optical portion, maintains a first quasi-stable configuration of the optical portion, maintains a second quasi-stable configuration of the optical portion, or a combination of any of the foregoing.

Aspect 50. The contact lens of aspect 49, wherein the tear fluid source comprises a tear fluid reservoir, a tear fluid depression, a tear meniscus, or a combination of any of the foregoing.

Aspect 51. The contact lens of aspect 49, wherein interaction is induced by a change in gaze angle, by interaction of an eyelid with the contact lens, or by a combination thereof.

Aspect 52. The contact lens of aspect 49, wherein interaction comprises fluidly coupling and fluidly decoupling the optical tear volume with a tear fluid source.

Aspect 53. The contact lens of aspect 49, wherein interaction comprises fluidly coupling and fluidly decoupling the optical tear volume with a tear meniscus.

Aspect 54. The contact lens of any one of aspects 43 to 53, wherein the at least one mechanism comprises one or more posterior grooves, wherein each of the one or more posterior grooves is disposed in the peripheral posterior surface.

Aspect 55. The contact lens of aspect 54, wherein at least one of the one or more posterior grooves intersects the circumference of the optical portion.

Aspect 56. The contact lens of any one of aspects 43 to 55, wherein the at least one mechanism is disposed within the peripheral portion, on the posterior surface of the peripheral portion, on the anterior surface of the peripheral portion, or a combination of any of the foregoing.

Aspect 57. The contact lens of any one of aspects 43 to 55, wherein the at least one mechanism comprises a protrusion on the peripheral anterior surface.

Aspect 58. The contact lens of any one of aspects 1 to 57, wherein, the contact lens comprises at least one fenestration connecting the peripheral posterior surface to the anterior posterior surface; and at least one of the fenestrations comprises a valve.

Aspect 59. The contact lens of aspect 58, wherein the valve comprises a capillary valve.

Aspect 60. The contact lens of any one of aspects 1 to 59, comprising one or more anterior grooves disposed in the peripheral anterior surface and one or more fenestrations connected to each of the one or more anterior grooves, wherein the at least one fenestration connects the anterior groove to the peripheral posterior surface.

Aspect 61. The contact lens of aspect 60, comprising a posterior groove disposed in the peripheral posterior surface and connected to at least one of the one or more fenestrations.

Aspect 62. The contact lens of aspect 61, wherein at least one of the one or more posterior grooves extends into the optical portion.

Aspect 63. The contact lens of any one of aspects 1 to 62, comprising: a plurality of radially disposed posterior grooves; and one or more fenestrations, wherein one or more fenestrations is coupled to each of the plurality of radially disposed posterior grooves.

Aspect 64. The contact lens of any one of aspects 1 to 63, comprising one or more depressions disposed in the anterior peripheral surface, and a fenestration coupled to each of the one or more depressions.

Aspect 65. The contact lens of aspect 64, wherein the fenestration is coupled to a posterior groove.

Aspect 66. The contact lens of any one of aspects 1 to 65, wherein the peripheral portion comprises a cavity disposed in the peripheral posterior surface.

Aspect 67. The contact lens of aspect 66, wherein the cavity is deformable upon interaction with an eyelid, motion of the eye, or a combination thereof.

Aspect 68. The contact lens of any one of aspects 1 to 67, wherein, the peripheral portion comprises a depression in the peripheral anterior surface; and a fenestration coupled to the depression; and a posterior groove coupled to the fenestration, wherein the posterior groove extends into the optical portion.

Aspect 69. A method of correcting vision, comprising wearing a contact lens of any one of aspects 1 to 68.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A contact lens comprising:
an optical portion, wherein the optical portion comprises an optical posterior base curvature; and an optical center;
a peripheral portion, wherein the peripheral portion comprises a peripheral posterior base curvature; and
a transition zone coupling the optical portion and the peripheral portion, wherein, when worn on the eye of a patient, the optical portion is characterized by a first quasi-stable configuration and a second quasi-stable configuration, wherein interaction of the contact lens with eye movement causes a transition between the first quasi-stable configuration and the second quasi-stable configuration,
wherein during the transition, a thickness of the optical portion does not change.

2. The contact lens of claim 1, wherein the optical portion comprises an optical posterior surface;
wherein the peripheral portion comprises a peripheral posterior surface;
wherein the contact lens is configured such that when worn on the eye of a patient, the optical portion can assume a plurality of configurations in response to a pressure applied to the optical portion;
wherein when a negative pressure is applied to the optical posterior surface, the optical posterior surface assumes one or more substantially conforming configurations with respect to the anterior surface of the cornea; and
wherein in the absence of a negative pressure, the optical posterior surface assumes a neutral configuration to provide a tear volume between the optical posterior surface and the anterior surface of the cornea.

3. The contact lens of claim 2, wherein the negative pressure is from 5 Pa to 1,500 Pa.

4. The contact lens of claim 2, wherein the negative pressure is from 10 Pa to 250 Pa.

5. The contact lens of claim 2, wherein,
the peripheral posterior base curvature is from 7.5 mm to 9.5 mm; and
the difference between the peripheral posterior base curvature and the optical posterior base curvature is greater than 0.1 mm.

6. The contact lens of claim 1, wherein the transition zone comprises one or more discontinuities extending across the transition zone.

7. The contact lens of claim 6, wherein the one or more discontinuities comprises one or more posterior grooves in the posterior surface of the peripheral portion and extending into the optical portion.

8. The contact lens of claim 7, wherein of the one or more posterior grooves are coupled to a fenestration.

9. The contact lens of claim 7, comprising one or more anterior grooves in the peripheral anterior surface.

10. The contact lens of claim 9, wherein the one or more anterior grooves are connected to one or more posterior grooves.

11. The contact lens of claim 9, wherein the one or more anterior grooves are not connected to one or more posterior grooves.

12. The contact lens of claim 1, wherein the eye movement comprises changing a gaze position of the eyeball or eyelids.

13. The contact lens of claim 1, wherein,
when worn on the eye of a patient, the optical portion is characterized by the first quasi-stable configuration and the second quasi-stable configuration;
the contact lens comprises one or more fenestrations connecting the peripheral posterior surface to the anterior surface; and
fluidly coupling one or more fenestrations to a tear meniscus causes a change in the optical power of an optical lens system comprising the optical portion of the contact lens, a tear film, and a lenticular optical tear volume.

14. The contact lens of claim 1, wherein the optical portion, the peripheral portion, or both the optical portion and the peripheral portion comprise at least one mechanism configured to transport tear fluid into and out of an optical tear volume formed between the optical posterior surface and the anterior surface of the cornea, when worn on an eye of a patient.

15. The contact lens of claim 14, wherein the transport of tear fluid into and out of the optical tear volume is associated with the transition between the first quasi-stable configuration of the optical portion and the second quasi-stable configuration of the optical portion.

16. The contact lens of claim 14, wherein the at least one mechanism comprises a posterior groove, an anterior groove, a fenestration, a tear fluid reservoir, a protrusion, a depression, a valve, a fenestration comprising a valve, a geometry of the optical portion, a geometry of the peripheral portion, or a combination of any of the foregoing.

17. The contact lens of claim 14, wherein interaction of tear fluid in the tear meniscus with the optical tear volume induces the transition between the first quasi-stable configuration of the optical portion and the second quasi-stable configuration of the optical portion, maintains the first quasi stable configuration of the optical portion, maintains the second quasi-stable configuration of the optical portion, or a combination of any of the foregoing.

18. The contact lens of claim 14, wherein motion of the eye, an eyelid, or a combination thereof, induces the transition between the first quasi-stable configuration of the optical portion and the second quasi-stable configuration of the optical portion, maintains the first quasi-stable configuration of the optical portion, maintains the second quasi-stable configuration of the optical portion, or a combination of any of the foregoing.

19. The contact lens of claim 14, wherein interaction of tear fluid in the tear meniscus with at least two of the optical portion, the peripheral portion, and the at least one mechanism, induces the transition between the first quasi-stable configuration of the optical portion and the second quasi-stable configuration of the optical portion, maintains the first quasi-stable configuration of the optical portion, maintains the second quasi-stable configuration of the optical portion, or a combination of any of the foregoing.

20. The contact lens of claim 1, wherein,
the contact lens comprises at least one fenestration connecting the peripheral posterior surface to the anterior surface; and
at least one of the fenestrations comprises a valve.

21. The contact lens of claim 20, wherein the valve comprises a capillary valve.

22. The contact lens of claim 1, comprising one or more anterior grooves disposed in the peripheral anterior surface and one or more fenestrations connected to each of the one or more anterior grooves, wherein the at least one fenestration connects the anterior groove to the peripheral posterior surface.

23. The contact lens of claim 1, comprising:
a plurality of radially disposed posterior grooves; and
one or more fenestrations, wherein one or more fenestrations is coupled to each of the plurality of radially disposed posterior grooves.

24. The contact lens of claim 1, comprising one or more depressions disposed in the anterior peripheral surface, and a fenestration coupled to each of the one or more depressions.

25. The contact lens of claim 1, wherein,
the peripheral portion comprises a depression in the peripheral anterior surface; and a fenestration coupled to the depression; and
a posterior groove coupled to the fenestration, wherein the posterior groove extends into the optical portion.

26. A contact lens comprising:
an optical portion, wherein the optical portion comprises an optical posterior base curvature and an optical center;
a peripheral portion, wherein the peripheral portion comprises a peripheral posterior base curvature;
a transition zone coupling the optical portion and the peripheral portion, wherein, when worn on the eye of a patient, the optical portion is characterized by a first quasi-stable configuration and a second quasi-stable configuration, wherein interaction of the contact lens with eye movement causes a transition between the first quasi-stable configuration and the second quasi-stable configuration; and
at least one fenestration connecting an peripheral posterior surface of the contact lens to an anterior surface of the contact lens,
wherein the at least one fenestration comprises a valve.

27. A contact lens comprising:
an optical portion, wherein the optical portion comprises an optical posterior base curvature and an optical center;
a peripheral portion, wherein the peripheral portion comprises a peripheral posterior base curvature; and
a transition zone coupling the optical portion and the peripheral portion, wherein, when worn on the eye of a patient, the optical portion is characterized by a first quasi-stable configuration and a second quasi-stable configuration, wherein interaction of the contact lens with eye movement causes a transition between the first quasi-stable configuration and the second quasi-stable configuration,
wherein the optical portion comprises an optical posterior surface;
wherein the peripheral portion comprises a peripheral posterior surface;
wherein the contact lens is configured such that when worn on the eye of a patient, the optical portion can assume a plurality of configurations in response to a pressure applied to the optical portion;
wherein when a negative pressure is applied to the optical posterior surface, the optical posterior surface assumes one or more substantially conforming configurations with respect to the anterior surface of the cornea; and
wherein in the absence of a negative pressure, the optical posterior surface assumes a neutral configuration to provide a tear volume between the optical posterior surface and the anterior surface of the cornea.

28. A contact lens comprising:
an optical portion, wherein the optical portion comprises an optical posterior base curvature; and an optical center;
a peripheral portion, wherein the peripheral portion comprises a peripheral posterior base curvature; and
a transition zone coupling the optical portion and the peripheral portion, wherein, when worn on the eye of a patient, the optical portion is characterized by a first quasi-stable configuration and a second quasi-stable configuration, wherein interaction of the contact lens with eye movement causes a transition between the first quasi-stable configuration and the second quasi-stable configuration,
wherein during the transition, an optical power of the optical portion does not change.
